(19) United States
(12) Patent Application Publication
(10) Pub. No.: US 2020/0017853 A1
(43) Pub. Date: Jan. 16, 2020

(54) COMPOSITIONS AND METHODS FOR PAIN AMELIORATION VIA MODIFICATION OF GENE EXPRESSION

(71) Applicant: Adynxx, Inc., San Francisco, CA (US)

(72) Inventors: Donald C. Manning, Bloomsbury, NJ (US); Scott Harris, San Francisco, CA (US); Kimberly Hebert, San Diego, CA (US); Dina Gonzalez, Reno, NV (US); Julien Mamet, San Francisco, CA (US); William Martin, Kensington, CA (US); Rick Orr, San Francisco, CA (US)

(21) Appl. No.: 16/080,547

(22) PCT Filed: Feb. 28, 2017
(86) PCT No.: PCT/US2017/019989

§ 371 Date: Aug. 28, 2018

Related U.S. Application Data
(60) Provisional application No. 62/421,456, filed on Nov. 14, 2016.
(60) Provisional application No. 62/301,272, filed on Feb. 29, 2016.

(51) Int. Cl. C12N 15/113; A61K 9/00; A61P 29/00; A61P 41/00

(52) U.S. Cl. CPC C12N 15/113; A61K 9/0019; C12N 2320/35; A61P 41/00; C12N 2310/13; A61P 29/00

(57) ABSTRACT

The present disclosure relates to oligonucleotide decoys, pharmaceutical compositions thereof, and the use of such to modulate nociceptive signaling and to prevent and/or treat pain.

### CROSS REFERENCE TO RELATED APPLICATIONS

This application is the U.S. national stage of International Application No. PCT/US2017/019989, filed on Feb. 28, 2017, which claims the benefit of priority to U.S. Provisional Application No. 62/301,272, filed on Feb. 29, 2016, and U.S. Provisional Application No. 62/421,456, filed on Nov. 14, 2016, the entire contents of each of which are hereby incorporated by reference in their entireties.

### DESCRIPTION OF THE TEXT FILE SUBMITTED ELECTRONICALLY

The contents of the text file submitted electronically herewith are incorporated herein by reference in their entirety: A computer readable format copy of the Sequence Listing (filename: ADDY_004_01WO_ST25.txt, date recorded: Feb. 27, 2017, file size ≈16 kilobytes).

### FIELD

The present disclosure relates to double-stranded nucleic acids, termed oligonucleotide decoys, pharmaceutical compositions thereof, and the use of such oligonucleotide decoys and pharmaceutical compositions to modulate nociceptive signaling and to prevent and/or treat pain.

### BACKGROUND

Pain may be defined as an unpleasant sensory and emotional experience associated with actual or potential tissue damage, or described in terms of such damage. Chronic pain afflicts 40% of the U.S. population and is associated with numerous deleterious medical conditions. Persistent and highly debilitating, chronic pain is generally accompanied by weakness, sleeplessness, a lack of appetite, irritability and depression. Over time, the quality of life is profoundly affected and patients are often incapable of accomplishing the simple tasks of everyday life.

Currently used pain treatments apply a three-step pain ladder which recommends the administration of drugs as follows: non-opioids (e.g., aspirin, acetaminophen, etc.), then, as necessary, mild opioids (e.g., codeine), and finally strong opioids (e.g., morphine). Despite this arsenal of drugs, over 50% of patients with chronic pain are not effectively treated.

The ineffectiveness of current pain treatments is, inter alia, due to significant toxicity issues with existing drug therapies. Mild to severe toxicity is induced by all classes of pain drugs: non steroidal inflammatory drugs cause gastro-intestinal damage, coxibs are associated with heart failure, and opioids are responsible for numerous side effects including respiratory depression, sedation, digestive malfunctions, and addiction. Furthermore, many of these drugs merely treat pain symptoms and do not effect the genomic basis underlying pain.

To this end, transcription factors are involved in multiple signaling pathways and frequently control the concurrent expression of numerous genes. Many transcription factors are involved in the regulation of the expression of genes that are involved in pain, including: POU factors, upstream stimulatory factors (USF), EGR1, cAMP-response element binding protein/activating transcription factors (CREB/ATF), activating protein 1 (AP1), serum response factor (SRF), promoter selective transcription factor (SP1), and the runt related transcription factor 1 (RUNX1).

Pain treatments that act upon the genomic basis of pain, such as by modulating gene expression via interaction with certain transcription factors associated with pain, would be a tremendous step forward compared to the aforementioned current pain treatment regimes. However, there is a current lack of such treatments available to the medical community.

### SUMMARY OF THE DISCLOSURE

The present disclosure addresses a crucial need in the art, by providing oligonucleotide decoys that modulate expression of genes associated with pain. To wit, the disclosure provides oligonucleotide decoys that target transcription factors associated with nociceptive pain signaling. The currently taught oligonucleotide decoys, and methods of utilizing the same, represent an advancement over currently prescribed pain medications, which do not treat the genomic basis of pain.

In one aspect, oligonucleotide decoys comprising one or more transcription factor binding sites are provided. In certain embodiments, each transcription factor binding site binds to a transcription factor selected from the group consisting of POU1F1, POU2F, POU3F, POU4F1, POU5F1, USF, EGR1, CREB/ATF, AP1, CEBP, SRF, ETS1, MEF2, SP1, RUNX, NFAT, ELK1, ternary complex factors, STAT, GATA1, ELF1, nuclear factor-granulocyte/macrophage a, HNF1, ZFHX3, IRF, TEAD1, TBP, NFY, caccc-box binding factors, KLF4, KLF7, IKZF, MAF, REST, HSF, KCNIP3 and PPAR transcription factors. In certain embodiments, the transcription factor that binds to a transcription factor binding site is a human transcription factor. In other embodiments, the transcription factor that binds to a transcription factor binding site is a non-human transcription factor (e.g., an avian, mammal (e.g., mouse, rat, dog, cat, horse, cow, etc.), or primate transcription factor).

In a related aspect, oligonucleotide decoys comprising two or more transcription factor binding sites are provided. In certain embodiments, each transcription factor binding site binds to a transcription factor selected from the group consisting of POU1F1, POU2F, POU3F, POU5F1, USF, EGR1, CREB/ATF, AP1, CEBP, SRF, ETS1, MEF2, SP1, RUNX, NFAT, ELK1, ternary complex factors, STAT, GATA1, ELF1, nuclear factor-granulocyte/macrophage a, POU4F1, HNF1, ZFHX3, IRF, TEAD1, TBP, NFY, caccc-box binding factors, KLF4, KLF7, IKZF, MAF, REST, HSF, KCNIP3 and PPAR transcription factors. In certain embodiments, the relative position of the two transcription factor binding sites within the decoy modulates (e.g., increases) the binding affinity between a transcription factor and its transcription factor binding site, as compared to the binding affinity between the transcription factor and a decoy having a single transcription factor binding site. In certain embodiments, the relative position of the two transcription factor binding sites within the decoy promotes dimerization of transcription factors bound to the sites.

In certain embodiments, the oligonucleotide decoys comprise: (a) a sequence selected from the group consisting of SEQ ID NOs.: 1-40, 42, 45 and 47-53; or (b) a sequence having at least 50% identity with a sequence selected from the group consisting of SEQ ID NOs.: 1-40, 42, 45 and 47-53.

In certain embodiments, the oligonucleotide decoys can be provided as salts, hydrates, solvates.

In another aspect, pharmaceutical compositions comprising oligonucleotide decoys are provided. The pharmaceutical compositions generally comprise one or more oligonucleotide decoys and a pharmaceutically acceptable vehicle.

In another aspect, methods for treating or preventing pain are provided. The methods generally involve administering to a patient in need of such treatment or prevention a therapeutically effective amount of an oligonucleotide decoy of the invention, or a pharmaceutical composition thereof. The current oligonucleotide decoys are capable of ameliorating acute pain and/or treating and/or preventing acute pain. The current oligonucleotide decoys are capable of ameliorating chronic pain.

In another aspect, methods for modulating the transcription of a gene in a cell involved in nociceptive signaling, such as a dorsal root ganglion and/or spinal cord neuron, are provided. The methods generally comprise administering to the cell an effective amount of an oligonucleotide decoy.

In another aspect, methods for modulating nociceptive signaling in a cell involved in nociceptive signaling, such as a dorsal root ganglion and/or spinal cord neuron, are provided. The methods generally comprise administering to the cell an effective amount of an oligonucleotide decoy.

In yet another aspect, methods for monitoring the proteolytic degradation of proteins involved in nociceptive signaling in a cell are provided. The methods generally comprise administering to the cell an effective amount of an oligonucleotide decoy.

Furthermore, the present disclosure is based in part, on the discovery that homeostatic levels of certain agents are important with respect to adverse effect(s) of a therapeutic entity, e.g., an active ingredient of a therapeutic entity. Accordingly, the present disclosure provides compositions or formulations capable of inhibiting or reducing adverse effect(s) of a therapeutic entity.

In one embodiment, the present disclosure provides a pharmaceutical composition, comprising: an active ingredient (e.g. an oligonucleotide decoy) and an in vivo stabilizing amount of an agent (e.g. a calcium ion), wherein the agent is associated with an adverse effect in vivo caused by the administration of the active ingredient without the agent, and wherein the in vivo stabilizing amount is the amount that substantially saturates the binding sites of the active ingredient to the agent. In aspects, the active ingredient is an oligonucleotide decoy comprising one or more binding sites for EGR1 and the agent is a calcium ion.

In another embodiment, the present disclosure provides a method of reducing an adverse effect of an active ingredient, comprising: administering the active ingredient (e.g. an oligonucleotide decoy) with an in vivo stabilizing amount of an agent (e.g. a calcium ion), wherein the agent is associated with the adverse effect of the active ingredient caused by the administration of the active ingredient without the agent, and wherein the in vivo stabilizing amount is the amount that substantially saturates the binding sites of the active ingredient to the agent. In aspects, the active ingredient is an oligonucleotide decoy comprising one or more binding sites for EGR1 and the agent is a calcium ion.

In another aspect, the disclosure provides a method for reducing acute pain, and/or preventing chronic pain, in a patient undergoing surgery, comprising: administering a single perioperative intrathecal injection of an effective amount of an oligonucleotide decoy, comprising one or more binding sites for EGR1, to a patient in need thereof. In aspects, the patient is undergoing a lower extremity surgery. In other aspects, the patient is undergoing an upper body surgery. In yet other aspects, the patient is undergoing a mid-body or abdominal surgery. In some other aspects, the patient is undergoing a knee surgery. In a certain aspect, the patient is undergoing a total knee arthroplasty. In certain embodiments, the intrathecal injection occurs at the L1/L2 lumbar interspace or below. In other embodiments, the intrathecal injection occurs at the L2/L3 lumbar interspace or below. In other embodiments, the intrathecal injection occurs at the L3/L4 lumbar interspace or below. In other embodiments, the intrathecal injection occurs at the L4/L5 lumbar interspace or below. In other embodiments, the intrathecal injection occurs at the L5/S1 lumbar interspace or below. In certain aspects, the oligonucleotide decoy is a synthetic phosphodiester duplex oligonucleotide sodium salt that is 23 base pairs or less in length. In a particular aspect, the oligonucleotide decoy comprises a nucleic acid sequence comprising a sense strand of 5′-GTATGCGTGGGCGGTGGGCGTAG-3′ and antisense strand of 3′-CATACGCACCCGCCACCCGCATC-5′. In embodiments, the oligonucleotide decoy comprises SEQ ID NO. 42. In certain embodiments, the effective amount of the oligonucleotide decoy is a concentration of about 110 mg/mL±25%. In aspects, the effective amount of the oligonucleotide decoy is from about 660 mg/6 mL to less than about 1100 mg/10 mL. In other aspects, the effective amount of the oligonucleotide decoy is less than about 1100 mg/10 mL. In yet other aspects, the effective amount of the oligonucleotide decoy is from about 500 mg/5 mL to about 700 mg/7 mL. In certain aspects, the effective amount of the oligonucleotide decoy is from about 330 mg/3 mL to about 660 mg/6 mL. In certain other aspects, the effective amount of the oligonucleotide decoy is about 660 mg/6 mL±25%. Further, in particular aspects, the effective amount of the oligonucleotide decoy is about 660 mg/6 mL. In embodiments, the patient experiences a statistically significant or clinically effective reduction in pain through at least day 28 post-surgery, or at least day 42 post-surgery, or at least day 90 post-surgery.

### BRIEF DESCRIPTION OF THE FIGURES

FIG. 1 illustrates NRS pain score when walking over time.

FIG. 2 illustrates NRS pain score when at rest over time.

FIG. 3 illustrates the percent of subjects with NRS pain score >2 at 42 days.

FIG. 4 is a flow diagram of the clinical study explained in the Examples.

FIG. 5 illustrates a responder analysis for pain when walking and at rest over time.

FIG. 6 illustrates that for AYX1 doses up to 660 mg/6 mL in humans the efficacy is optimal, but at a dose of 1100 mg in 10 mL the efficacy decreases, as also seen in the animal data.

FIG. 7 is a graphical representation of pain manifestation, illustrating that a new pattern of protein expression is required for sustained neuronal sensitization following trauma or surgery.

FIG. 8 demonstrates the role that EGR1 plays in pain establishment and maintenance. AYX1, an oligonucleotide decoy encompassed by SEQ ID NO. 42, preferentially binds EGR1.

FIG. 9 is a schematic of the human spine and illustrates the baracity of AYX1. In the Examples section, the inventors demonstrate a significant effect utilizing the 660 mg/6 mL dosage form, which was independent of injection site.

FIG. 10 illustrates the study design explained in the Examples.

FIG. 11 demonstrates a significant reduction in post-operative pain achieved by the taught oligonucleotide decoy AYX1 at 660 mg/6 mL at days 7-28. Further, the table illustrates that pain was reduced by 31% on days 7-28 utilizing the 660 mg/6 mL dosage.

FIG. 12 demonstrates the statistical significance of the treatment effect is maintained up to day 42.

FIG. 13 demonstrates the magnitude of treatment effect is equivalent across injection sites.

FIG. 14 demonstrates that similar to the pain with walking results, AYX1's effect on pain at rest maintains statistical significance over longer time periods up to at least 42 days.

FIG. 15 is a comparison of episodic post-operative analgesic assessments to the presently taught preventative therapy assessments.

FIG. 16 is a graphical comparison of episodic post-operative analgesic assessments to the presently taught preventative therapy assessments.

FIG. 17 demonstrates that in the pain when walking category a higher percentage of subjects treated with only standard of care (SOC) consistently reported higher pain scores than subjects treated withy AYX1.

FIG. 18 demonstrates that in the pain when at rest category AYX1 treated subjects consistently reported lower pain scores earlier than subjects treated with placebo and SOC.

FIG. 19 demonstrates that opioid utilization was similar across treatment groups.

### DETAILED DESCRIPTION OF THE DISCLOSURE

Following long-standing patent law convention, the terms “a”, “an”, and “the” refer to “one or more” when used in this application, including the claims, unless clearly indicated otherwise.

Throughout this application, the term “about” is used to indicate that a value includes the standard deviation of error for the device or method being employed to determine the value, e.g. ±10%.

“Acute” refers to a period of time that is shorter than “chronic.” Acute pain is where pain symptoms appear suddenly and do not extend beyond healing of the underlying injury. In embodiments, acute pain can be measured in hours or even days. Thus, the methods and compositions of the disclosure are able to treat acute pain

“Binding,” as used in the context of transcription factors binding to oligonucleotide decoys, refers to a direct interaction (e.g., non-covalent bonding between the transcription factor and oligonucleotide decoy, including hydrogen-bonding, van der Waals bonding, etc.) between at least one transcription factor and an oligonucleotide decoy. Accordingly, an oligonucleotide that does not bind to a transcription factor does not directly interact with said transcription factor.

“Chronic” refers to a period of time that is longer than “acute.” Chronic pain, unlike acute pain, is a process that lasts for a long period of time. In some embodiments, chronic is a period of time comprising months (e.g., at least 1, 2, 3, 4, 5, 6, 7, 8, 9, 10, 11, and 12 months) or years. In particular embodiments, “chronic pain” refers to pain that lasts 3 months or more in a patient. Thus, the methods and compositions of the disclosure are able to treat chronic pain, i.e. pain that lasts 3 months or more.

“Compounds” in some aspects, refers to double-stranded oligonucleotides, also referred to herein as oligonucleotide decoys. The compounds described herein may contain one or more chiral centers and/or double bonds and therefore, may exist as stereoisomers, such as double-bond isomers (i.e., geometric isomers), enantiomers or diastereomers. Accordingly, the chemical structures depicted herein encompass all possible enantiomers and stereoisomers of the illustrated compounds including the stereoisomerically pure form (e.g., geometrically pure, enantiomerically pure or diastereomerically pure) and enantiomeric and stereoisomeric mixtures. Enantiomeric and stereoisomeric mixtures can be resolved into their component enantiomers or stereoisomers using separation techniques or chiral synthesis techniques well known to the skilled artisan. Compounds may also exist in several tautomeric forms including the enol form, the keto form and mixtures thereof. Accordingly, the chemical structures depicted herein encompass all possible tautomeric forms of compounds. Compounds described herein also include isotopically labeled compounds where one or more atoms have an atomic mass different from the atomic mass conventionally found in nature. Examples of isotopes that may be incorporated into the compounds of the invention include, but are not limited to, ²H, ³H, ¹¹C, ¹³C, ¹⁴C, ¹⁵N, ¹⁸O, ¹⁷O, etc. Compounds may exist in unsolvated forms as well as solvated forms, including hydrated forms and as N-oxides. In general, compounds may be hydrated, solvated or N-oxides. Certain compounds may exist in multiple crystalline or amorphous forms. All physical forms are equivalent for the uses contemplated herein. Further, it should be understood, when partial structures of the compounds are illustrated, that brackets indicate the point of attachment of the partial structure to the rest of the molecule.

As used herein, the term “effective” (e.g., “an effective amount”) means adequate to accomplish a desired, expected, or intended result. An effective amount can be a therapeutically effective amount. A “therapeutically effective amount” refers to the amount of an active ingredient (e.g. an oligonucleotide decoy) that, when administered to a subject, is sufficient to effect such treatment of a particular disease or condition (e.g. pain). The “therapeutically effective amount” will vary depending on the active ingredient, the disease or condition, the severity of the disease or condition, and the age, weight, etc., of the subject to be treated.

The terms “minimizing,” “inhibiting,” and “reducing,” or any variation of these terms, includes any measurable decrease or complete inhibition or reduction to achieve a desired result. For example, there may be a decrease of 5%, 10%, 15%, 20%, 25%, 30%, 35%, 40%, 45%, 50%, 55%, 60%, 65%, 70%, 75%, 80%, 85%, 90%, 95%, 99%, or more, or any range derivable therein, reduction of activity compared to normal.

“Modulation of gene expression level” refers to any change in gene expression level, including an induction or activation (e.g., an increase in gene expression), an inhibition or suppression (e.g., a decrease in gene expression), or a stabilization (e.g., prevention of the up-regulation or down-regulation of a gene that ordinarily occurs in response to a stimulus, such as a pain-inducing stimulus).

“Nociceptive signaling” refers to molecular and cellular mechanisms involved in the detection of a noxious stimulus or of a potentially harmful stimulus, which leads to the perception of pain, including neurotransmitter synthesis and release, neurotransmitter-induced signaling, membrane depolarization, and related intra-cellular and inter-cellular signaling events.

An “oligonucleotide decoy” refers to any double-stranded, nucleic acid-containing polymer generally less than approximately 200 nucleotides (or 100 base pairs) and including, but not limited to: DNA, RNA and RNA-DNA hybrids.

The term encompasses sequences that include any of the known base analogs of DNA and RNA including, but not limited to, 2,6-diaminopurine, 5-carboxymethylaminomethyl-2-thiouracil, 5-carboxymethylaminomethyluracil, dihydrouracil, inosine, uracil-5-oxyacetic acid, N6-isopentenyladenine, 1-methyladenine, N-uracil-5-oxyacetic acid methylester, queosine, 2-thiocytosine, 5-bromouracil, methylphosphonate, phosphorodithioate, ormacetal, 3′-thioformacetal, nitroxide backbone, sulfone, sulfamate, morpholino derivatives, locked nucleic acid (LNA) derivatives, or peptide nucleic acid (PNA) derivatives. In some embodiments, the oligonucleotide decoy is composed of two complementary single-stranded oligonucleotides that are annealed together. In other embodiments, the oligonucleotide decoy is composed of one single-stranded oligonucleotide that forms intramolecular base pairs to create a substantially double-stranded structure.

“Pain” refers to an unpleasant sensory and emotional experience that is associated with actual or potential tissue damage or described in such terms. All of the different manifestations and qualities of pain, including mechanical pain (e.g., induced by a mechanical stimulus or by body motion), temperature-induced pain (e.g., pain induced by hot, warm and/or cold temperatures), and chemically-induced pain (e.g., pain induced by a chemical). In certain embodiments, pain is chronic, sub-chronic, acute, or sub-acute. In certain embodiments, pain features hyperalgesia (i.e., an increased sensitivity to a painful stimulus) and/or allodynia (i.e., a painful response to a usually non-painful stimulus). In certain embodiments, pain is pre-existing in a patient. In other embodiments, pain is iatrogenic, induced in a patient (e.g., post-operative pain).

“Pharmaceutically acceptable salt” refers to a salt of a compound, which possesses the desired pharmacological activity of the parent compound. Such salts include, but are not limited to: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced by a metal ion, e.g., an alkali metal ion, an alkaline earth ion, or an aluminum ion; or coordinates with an organic base such as ethanolamine, diethanolamine, triethanolamine, N-methylglucamine and the like.

“Pharmaceutically acceptable vehicle” refers to a diluent, adjuvant, excipient or carrier with which a compound of the invention is administered.

“Patient” includes any animal, including birds, mammals, primates, and humans.

“Preventing” or “prevention” refers to (1) a reduction in the risk of acquiring a disease or disorder (e.g., causing at least one of the clinical symptoms of a disease not to develop in a patient that may be exposed to or predisposed to the disease, but does not yet experience or display symptoms of the disease), or (2) a reduction in the likely severity of a symptom associated with a disease or disorder (e.g., reducing the likely severity of at least one of the clinical symptoms of a disease in a patient that may be exposed to or predisposed to the disease, but does not yet experience or display symptoms of the disease).

“Treating” or “treatment” of any condition, disease, or disorder refers, in some embodiments, to ameliorating the condition, disease, or disorder (i.e., arresting or reducing the development of the disease or at least one of the clinical symptoms thereof). In other embodiments “treating” or “treatment” refers to ameliorating at least one physical parameter, which may not be discernible by the patient. In yet other embodiments, “treating” or “treatment” refers to inhibiting the condition, disease, or disorder, either physically, (e.g., stabilization of a discernible symptom), physiologically, (e.g., stabilization of a physical parameter) or both. In yet other embodiments, “treating” or “treatment” refers to delaying the onset of the condition, disease, or disorder.

“Therapeutically effective amount” means the amount of a compound that, when administered to a patient, is sufficient to effect such treatment of a particular disease or condition. The “therapeutically effective amount” will vary depending on the compound, the disease, the severity of the disease, and the age, weight, etc., of the patient to be treated. In certain aspects, the “therapeutically effective amount” refers to the amount of an oligonucleotide decoy.

### Pharmaceutical Compositions

The pharmaceutical compositions disclosed herein comprise a therapeutically effective amount of one or more oligonucleotide decoys, preferably, in purified form, together with a suitable amount of a pharmaceutically acceptable vehicle, so as to provide a form for proper administration to a patient. When administered to a patient, oligonucleotide decoys and pharmaceutically acceptable vehicles are preferably sterile. Water can be a vehicle when oligonucleotide decoys are administered intravenously. Saline solutions and aqueous dextrose and glycerol solutions can also be employed as liquid vehicles, particularly for injectable solutions. Suitable pharmaceutical vehicles include excipients such as starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present pharmaceutical compositions, if desired, can also contain minor amounts of wetting or emulsifying agents, or pH buffering agents. In addition, auxiliary, stabilizing, thickening, lubricating and coloring agents may be used.

Pharmaceutical compositions may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries, which facilitate processing of compounds disclosed herein into preparations which can be used pharmaceutically. Proper formulation is dependent upon the route of administration chosen.

The present pharmaceutical compositions can take the form of solutions, suspensions, emulsions, tablets, pills, pellets, capsules, capsules containing liquids, powders, sustained-release formulations, suppositories, aerosols, sprays, suspensions, or any other form suitable for use. Other examples of suitable pharmaceutical vehicles have been described in the art (see Remington's Pharmaceutical Sciences, Philadelphia College of Pharmacy and Science, 19th Edition, 1995).

Pharmaceutical compositions for oral delivery may be in the form of tablets, lozenges, aqueous or oily suspensions, granules, powders, emulsions, capsules, syrups, or elixirs, for example. Orally administered compositions may contain one or more optional agents, for example, sweetening agents such as fructose, aspartame or saccharin, flavoring agents such as peppermint, oil of wintergreen, or cherry coloring agents and preserving agents, to provide a pharmaceutically palatable preparation. Moreover, when in tablet or pill form, the compositions may be coated to delay disintegration and absorption in the gastrointestinal tract, thereby providing a sustained action over an extended period of time. Oral compositions can include standard vehicles such as mannitol, lactose, starch, magnesium stearate, sodium saccharine, cellulose, magnesium carbonate, etc. Such vehicles are preferably of pharmaceutical grade.

For oral liquid preparations such as, for example, suspensions, elixirs and solutions, suitable carriers, excipients or diluents include water, saline, alkyleneglycols (e.g., propylene glycol), polyalkylene glycols (e.g., polyethylene glycol), oils, alcohols, slightly acidic buffers between pH 4 and pH 6 (e.g., acetate, citrate, or ascorbate at between about 5 mM to about 50 mM), etc. Additionally, flavoring agents, preservatives, coloring agents, bile salts, acylcarnitines and the like may be added.

Compositions for administration via other routes may also be contemplated. For buccal administration, the compositions may take the form of tablets, lozenges, etc., formulated in conventional manner. Liquid drug formulations suitable for use with nebulizers and liquid spray devices and EHD aerosol devices will typically include a compound with a pharmaceutically acceptable vehicle. The pharmaceutically acceptable vehicle may be a liquid such as alcohol, water, polyethylene glycol or a perfluorocarbon. Optionally, another material may be added to alter the aerosol properties of the solution or suspension of compounds. This material may be liquid such as an alcohol, glycol, polyglycol or a fatty acid. Other methods of formulating liquid drug solutions or suspension suitable for use in aerosol devices are known to those of skill in the art (see, e.g., Biesalski, U.S. Pat. No. 5,112,598; Biesalski, U.S. Pat. No. 5,556,611).

A compound may be formulated for intrathecal injection

A compound may be formulated for delivery using ultrasound-release methods.

A compound may also be formulated in rectal or vaginal compositions such as suppositories or retention enemas, e.g., containing conventional suppository bases such as cocoa butter or other glycerides.

In addition to the formulations described previously, a compound may also be formulated as a depot preparation. Such long acting formulations may be administered by implantation (for example, subcutaneously or intramuscularly) or by intramuscular injection. Thus, for example, a compound may be formulated with suitable polymeric or hydrophobic materials (for example, as an emulsion in an acceptable oil) or ion exchange resins, or as sparingly soluble derivatives, for example, as a sparingly soluble salt.

An oligonucleotide decoy may be included in any of the above-described formulations, or in any other suitable formulation, as a pharmaceutically acceptable salt, a solvate or hydrate. Pharmaceutically acceptable salts substantially retain the activity of the parent compound and may be prepared by reaction with appropriate bases or acids and tend to be more soluble in aqueous and other protic solvents than the corresponding parent form.

According to the present invention, the composition of the present invention can further comprise a buffer. Any suitable buffer can be used for the composition of the present invention. In some other embodiments, the buffer system used for the composition of the present invention is an organic or inorganic buffer. Examples of buffers include phosphate buffers, citrate buffers, borate buffers, bicarbonate buffers, carbonate buffers, acetate buffers, ammonium buffers, and tromethamine (Tris) buffers.

According to the present invention, in some embodiments, when the active ingredient is an oligonucleotide and the agent is an ion, e.g., calcium, the buffer is a non-phosphate based buffer. The amount of buffer employed will be ascertainable to a skilled artisan, such as an amount ranging from 0.01 mM to 1 M, such as 10 mM.

Intrathecal administration is a route of administration to deliver drugs through the spinal sac and directly into the cerebrospinal fluid (CSF).

### Therapeutic Uses

In certain embodiments, an oligonucleotide decoy and/or pharmaceutical composition thereof is administered to a patient, such as an animal (e.g., a bird, mammal, primate, or human), suffering from pain including, but not limited to: mechanical pain (e.g., mechanical hyperalgesia and/or allodynia), chemical pain, temperature pain, chronic pain, sub-chronic pain, acute pain, sub-acute pain, inflammatory pain, neuropathic pain, muscular pain, skeletal pain, post-surgery pain, arthritis pain, and diabetes pain. Further, in certain embodiments, the oligonucleotide decoys and/or pharmaceutical compositions thereof are administered to a patient, such as an animal, as a preventative measure against pain including, but not limited to: post-operative pain, chronic pain, inflammatory pain, neuropathic pain, muscular pain, and skeletal pain. In certain embodiments, the oligonucleotide decoy(s) and/or pharmaceutical compositions thereof may be used for the prevention and/or treatment and/or amelioration of one facet of pain while concurrently treating another symptom of pain.

Thus, in certain embodiments, the disclosure provides methods of treating or preventing pain in a patient comprising administering to a patient suffering from pain a therapeutically effective amount of an oligonucleotide decoy described herein. In related embodiments, methods of preventing pain in a patient are provided. Such methods comprise administering to a patient in need thereof (e.g., a patient likely to develop pain, e.g., post-operative pain) a therapeutically effective amount of an oligonucleotide decoy described herein. In certain embodiments, the oligonucleotide decoy is administered perineurally, epidurally/peridurally, intrathecally.

In certain embodiments, the invention provides methods for treating or preventing pain in a patient comprising administering to a patient in need thereof a therapeutically effective amount of an oligonucleotide decoy, wherein the oligonucleotide decoy does not bind to the transcription factors AP1, ETS1 and STAT. In other embodiments, the invention provides methods for treating or preventing pain in a patient comprising administering to the patient in need thereof a therapeutically effective amount of one or more oligonucleotide decoys, wherein the oligonucleotide decoys bind to one or more transcription factors selected from the group consisting of EGR1, AP1, ETS1, GATA and STAT transcription factors, provided that the pain is not lower back pain due to an intervertebral disc disorder.

In certain embodiments, the invention provides methods for modulating transcription of a gene present in a cell involved in nociceptive signaling and/or the perception of pain in a patient. In certain embodiments, modulation comprises suppressing or repressing gene expression. In other embodiments, modulation comprises stabilizing gene expression. In still other embodiments, modulation comprises activating or inducing gene expression. In certain embodiments, the gene is involved in nociceptive signaling. Genes involved in nociceptive signaling include, but are not limited to: genes encoding membrane proteins (e.g., ion channels, membrane receptors, etc.), soluble signaling molecules (e.g., intracellular signaling molecules or neurotransmitters), synthetic enzymes (e.g., neurotransmitter synthesis enzymes), and transcription factors (e.g. EGR1). Specific examples of such genes include, but are not limited to: BDKRB2, HTR3A, SCN9A, BDNF, GRA15, NOS1, GCH1, CDK5R1, CACNA1B, P2XR3 and PNMT.

In other embodiments, the invention provides methods for modulating nociceptive signaling in a cell. In certain embodiments, modulation comprises suppressing or repressing nociceptive signaling. In certain embodiments, modulating nociceptive signaling in a cell comprises modulating, e.g., increasing, proteolysis of a protein involved in nociceptive signaling in said cell. For instance, abnormally high proteasome activity has been linked to strong deficits of neuronal plasticity (i.e., a major cellular feature of pain). EGR1 is known to repress the expression of selected proteasome factors, thus limiting EGR1-dependent nociceptive signaling activity is relevant for treating pain. Further, neutrophines activate specific receptors in pain neurons that trigger nociceptive signaling. USF factors activate the expression of CGRP and Substance P, two major neurotrophins capable of inducing pain. Inhibiting USF factors is a potential approach to inhibit nociceptive signaling. In certain embodiments, modulation comprises activation of an inhibitor of nociceptive signaling.

In still other embodiments, the invention provided methods for modulating, e.g., increasing, proteolytic degradation of a protein involved in nociceptive signaling in a cell. In certain embodiments, modulation of protein degradation comprises stimulating proteosome function. In certain embodiments, the protein is involved in nociceptive signaling. Proteins involved in nociceptive signaling include, but are not limited to membrane proteins (e.g., ion channels, membrane receptors, etc.), soluble signaling molecules (e.g., intracellular signaling molecules or neurotransmitters), synthetic enzymes (e.g., neurotransmitter synthesis enzymes), and transcription factors. Specific examples of such proteins include, but are not limited to, BDKRB2, HTR3A, SCN9A, BDNF, GRM5, NOS1, GCH1, CDK5R1, CACNA1B, P2XR3 and PNMT.

In certain embodiments, the cell of the various methods is in vivo (e.g., in a patient suffering from pain or likely to suffer from pain). A cell in vivo can be located in different locations including, but not limited to, a dorsal root ganglia and/or the spinal cord. In other embodiments, the cell of the various methods is provided in vitro (e.g., in a petri dish). The cell can be any cell involved in nociceptive signaling, including, but not limited to, a neuron (e.g., a pain neuron from dorsal root ganglia and/or the spinal cord or from the sympathetic nervous system), a glial cell, a tissue supportive cell (e.g., fibroblast), an immune cell, or a cell from a cell line (e.g., a PC12 cell).

In still some embodiments, the active ingredient is an oligonucleotide decoy including one or more binding sites for EGR1 and the composition of the present invention comprising the active ingredient can be used to treat, pre-treat, or prevent pain or related conditions. All of the different manifestations and qualities of pain, including mechanical pain (e.g., induced by a mechanical stimulus or by body motion; mechanical hyperalgesia or allodynia), temperature-induced pain (e.g., pain induced by hot, warm or cold temperatures), and chemically-induced pain (e.g., pain induced by a chemical) are included. In certain embodiments, pain is chronic, sub-chronic, acute, or sub-acute. In certain embodiments, pain features hyperalgesia (i.e., an increased sensitivity to a painful stimulus) or allodynia (i.e., a painful response to a usually non-painful stimulus). Pain can be inflammatory pain, neuropathic pain, muscular pain, skeletal pain, post-surgery pain, arthritis pain, or diabetes pain. In certain embodiments, pain is pre-existing in a patient. In other embodiments, pain is iatrogenic, induced in a patient (e.g., post-operative pain).

In some other embodiments, pain or pain related conditions include post-operative pain, chronic pain, inflammatory pain, neuropathic pain, muscular pain, and skeletal pain. In certain embodiments, compositions can be used for the prevention of one facet of pain while concurrently treating another symptom of pain.

### Methods of Administration and Dosage

The present methods for treatment or prevention of pain require administration of an oligonucleotide decoy, or pharmaceutical composition thereof, to a patient in need of such treatment or prevention. The compounds and/or pharmaceutical compositions thereof may be administered by any convenient route, for example, by infusion or bolus injection, by absorption through epithelial or mucocutaneous linings (e.g., oral mucosa, rectal and intestinal mucosa, etc.), or orally. Administration can be systemic or local. Various delivery systems are known, including, e.g., encapsulation in liposomes, microparticles, microcapsules, capsules, etc., that can be used to administer a compound and/or pharmaceutical composition thereof.

Methods of administration include, but are not limited to, intradermal, intramuscular, intraperitoneal, intravenous, subcutaneous, intranasal, epidural/peridural, intrathecal, oral, sublingual, intranasal, intracerebral, intravaginal, transdermal, rectally, by inhalation or topically, particularly to the ears, nose, eyes, or skin. In certain embodiments, more than one oligonucleotide decoy is administered to a patient. The mode of administration will depend in-part upon the site of the medical condition.

In specific embodiments, it may be desirable to administer one or more oligonucleotide decoys locally to the area in need of treatment. This may be achieved, for example, and not by way of limitation, by local infusion during surgery, topical application (e.g., in conjunction with a wound dressing after surgery), by injection, by means of a catheter, by means of a suppository, or by means of an implant, said implant being of a porous, non-porous, or gelatinous material, including membranes, such as sialastic membranes, or fibers. In some embodiments, administration can be by direct injection at the site (e.g., former, current, or expected site) of pain.

In certain embodiments, it may be desirable to introduce one or more oligonucleotide decoys into the nervous system by any suitable route, including but not restricted to intraventricular, intrathecal, perineural and/or epidural/peridural injection. Intraventricular injection may be facilitated by an intraventricular catheter, for example, attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant.

The amount of oligonucleotide decoy that will be effective in the treatment or prevention of pain in a patient will depend on the specific nature of the condition and can be determined by standard clinical techniques known in the art. In addition, in vitro or in vivo assays may optionally be employed to help identify optimal dosage ranges. The amount of a oligonucleotide decoy administered will, of course, be dependent on, among other factors, the subject being treated, the weight of the subject, the severity of the affliction, the manner of administration, and the judgment of the prescribing physician. In certain embodiments, a single dose of oligonucleotide decoy comprises about 1 mg to about 3000 mg, 1 mg to about 2000 mg, 1 mg to about 1500 mg, 1 mg to about 1200 mg, 1 mg to about 1100 mg, 100 mg to about 3000 mg, 100 mg to about 2000 mg, 100 mg to about 1500 mg, 100 mg to about 1200 mg, 100 mg to about 1100 mg, 200 mg to about 3000 mg, 200 mg to about 2000 mg, 200 mg to about 1500 mg, 200 mg to about 1200 mg, 200 mg to about 1100 mg, 300 mg to about 3000 mg, 300 mg to about 2000 mg, 300 mg to about 1500 mg, 300 mg to about 1200 mg, 300 mg to about 1100 mg, 400 mg to about 3000 mg, 400 mg to about 2000 mg, 400 mg to about 1500 mg, 400 mg to about 1200 mg, 400 mg to about 1100 mg, 500 mg to about 3000 mg, 500 mg to about 2000 mg, 500 mg to about 1500 mg, 500 mg to about 1200 mg, 500 mg to about 1100 mg, 600 mg to about 3000 mg, 600 mg to about 2000 mg, 600 mg to about 1500 mg, 600 mg to about 1200 mg, 600 mg to about 1100 mg, 700 mg to about 3000 mg, 700 mg to about 2000 mg, 700 mg to about 1500 mg, 700 mg to about 1200 mg, 700 mg to about 1100 mg, 800 mg to about 3000 mg, 800 mg to about 2000 mg, 800 mg to about 1500 mg, 800 mg to about 1200 mg, 800 mg to about 1100 mg, 900 mg to about 3000 mg, 900 mg to about 2000 mg, 900 mg to about 1500 mg, 900 mg to about 1200 mg, 900 mg to about 1100 mg, of oligonucleotide decoy per patient. Further, one embodiment may comprise administering 1100 mg±500 mg of oligonucleotide decoy per patient. Or, 1100 mg±400 mg of oligonucleotide decoy per patient. Or, 1100 mg±300 mg of oligonucleotide decoy per patient. Or, 1100 mg±200 mg of oligonucleotide decoy per patient. Or, 1100 mg±100 mg of oligonucleotide decoy per patient. Or, 1100 mg±50 mg of oligonucleotide decoy per patient. Or, 1100 mg±10 mg of oligonucleotide decoy per patient. Or, 1100 mg±50% of oligonucleotide decoy per patient. Or, 1100 mg±40% of oligonucleotide decoy per patient. Or, 1100 mg±30% of oligonucleotide decoy per patient. Or, 1100 mg±20% of oligonucleotide decoy per patient. Or, 1100 mg±10% of oligonucleotide decoy per patient. Or, 1100 mg±5% of oligonucleotide decoy per patient.

In certain embodiments, a single dose of oligonucleotide decoy comprises about: 100 mg to about 700 mg, 150 mg to about 700 mg, 200 mg to about 700 mg, 250 mg to about 700 mg, 300 mg to about 700 mg, 350 mg to about 700 mg, 400 mg to about 700 mg, 450 mg to about 700 mg, 500 mg to about 700 mg, 550 mg to about 700 mg, 600 mg to about 700 mg, or 650 mg to about 700 mg. Further, one embodiment may comprise administering 660 mg±330 mg of oligonucleotide decoy per patient. Or, 660 mg±260 mg of oligonucleotide decoy per patient. Or, 660 mg±200 mg of oligonucleotide decoy per patient. Or, 660 mg±130 mg of oligonucleotide decoy per patient. Or, 660 mg±60 mg of oligonucleotide decoy per patient. Or, 660 mg±30 mg of oligonucleotide decoy per patient. Or, 660 mg±10 mg of oligonucleotide decoy per patient. Or, 660 mg±50% of oligonucleotide decoy per patient. Or, 660 mg±40% of oligonucleotide decoy per patient. Or, 660 mg±30% of oligonucleotide decoy per patient. Or, 660 mg±20% of oligonucleotide decoy per patient. Or, 660 mg±10% of oligonucleotide decoy per patient. Or, 660 mg±5% of oligonucleotide decoy per patient. Or, 660 mg±1% of oligonucleotide decoy per patient.

In aspects, the dosage forms may be administered to a patient once per day. Dosing may be provided alone or in combination with other drugs and may continue as long as required for effective treatment or prevention of pain.

### Combination Therapy

In certain embodiments, oligonucleotide decoys and/or pharmaceutical compositions thereof can be used in combination therapy with at least one other therapeutic agent, which may include, but is not limited to, an oligonucleotide decoy. The oligonucleotide decoy and/or pharmaceutical composition thereof and the therapeutic agent can act additively or, more preferably, synergistically. In some embodiments, an oligonucleotide decoy and/or a pharmaceutical composition thereof is administered concurrently with the administration of another therapeutic agent, including another oligonucleotide decoy. In other embodiments, an oligonucleotide decoy or a pharmaceutical composition thereof is administered prior or subsequent to administration of another therapeutic agent, including another oligonucleotide decoy.

### Formulations of an Oligonucleotide Decoy and a Stabilizing Agent

The present disclosure is based, in part, on the discovery that homeostatic levels of certain agents are important with respect to adverse effect(s) of a therapeutic entity, e.g., an active ingredient of a therapeutic entity. Accordingly the present invention provides compositions or formulations capable of inhibiting or reducing adverse effect(s) of a therapeutic entity. In addition, the present invention also provides methods of using the compositions or formulations for therapeutic treatments.

In one aspect, the present invention provides a composition, such as a pharmaceutical composition, comprising an active ingredient and an agent associated, directly or indirectly, with one or more adverse effect(s) of the active ingredient. In one embodiment, the agent is any entity, of which the homeostatic levels are directly or indirectly related to one or more adverse effect(s) of the active ingredient. In another embodiment, the agent is any entity, of which the homeostatic levels are changed, e.g., substantially upon administration of the active ingredient in vivo. In yet another embodiment, the agent is any entity, of which the homeostatic levels are sensitive to the administration of the active ingredient in vivo. In still another embodiment, the agent is any entity which is capable of interacting or interacts, directly or indirectly, with the active ingredient. In still yet another embodiment, the agent is any entity which is capable of binding or binds, directly or indirectly, with the active ingredient.

According to the present invention, the agent can be different, e.g., even with respect to the same active ingredient, depending on the tissue or cell type the active ingredient is administered into. In some embodiments, the agent is an ion. An ion can be an organic acid, such as malic, ascorbic, tartaric, lactic, acetic, formic, oxalic, or citric acid. In some embodiments, the agent is a metal ion, e.g., iron, zinc, copper, lead and nickel, etc. In some embodiments, the agent has a charge that is opposite of the net charge of the active ingredient. In some embodiments, the agent is a cation or anion. In some other embodiments, the agent is a calcium ion, a magnesium ion, or a potassium ion. In some other embodiments, the agent is an ion, carbohydrate (e.g., sugars, starches, etc.), lipid (e.g., saturated fatty acids, unsaturated fatty acids, triacylglycerols, glycerophospholipids, sphingolipids, and cholesterol, etc.), vitamin (e.g., selenium, zinc, vitamin A, thiamine, riboflavin, pyridoxin, niacin, pantothenic acid, cyanocobalamin, L-ascorbic acid and α-tocopherol, etc.), or alcohol (e.g., polyols such as glucose and mannitol, as well as, e.g., ethanol, etc.) or a combination thereof.

In yet further embodiments, the agent with respect to cerebrospinal fluid is an ion, e.g., calcium ions, magnesium ions or potassium ions.

In still some other embodiments, the agent with respect to blood is one or more blood electrolytes and/or major constituents of extracellular, cellular and interstitial fluids. In some exemplary embodiments, the agent with respect to blood is Na⁺, K⁺, Ca²⁺, Mg²⁺, Cl⁻, bicarbonates (e.g., HCO₃⁻), phosphorus (e.g., HPO₄²⁻), sulfates (e.g., SO₄²⁻), organic acid, proteins, metal ions (iron, zinc, copper, lead and nickel, etc.), carbohydrates or alcohols (e.g., glucose, mannitol, ethanol), lipids, vitamins (e.g., selenium, zinc) or any combination thereof.

According to the present invention, the agent used in the composition of the active ingredient can be any amount suitable for the administration of the active ingredient in vivo, e.g., any amount that either inhibits or decreases one or more adverse effect(s) of the active ingredient without the agent.

According to the present invention, one or more adverse effect(s) of the active ingredient includes any unwanted or undesirable effect produced as a result of in vivo administration of the active ingredient. An adverse effect can be any long term or short effect, local or systematic effect, or any effect associated with the toxicity of the active ingredient. Exemplary adverse effects include pain, headache, vomiting, arrhythmia, shivering, respiratory depression, dizziness, loss of motor control, lack of coordination, fatigue, memory impairment, rash, or numbness. In one embodiment, the adverse effect in the context of pain treatment with an oligonucleotide decoy can be relatively minor (e.g., light tail movement in a rodent or dog animal model) or more severe (e.g., a seizure), or may include muscle trembling, increased muscle tone in a limb, whole body rigidity, pain, or spontaneous vocalization.

In one embodiment, the agent used in the composition of the active ingredient is an in vivo stabilizing amount. As used herein, an “in vivo stabilizing amount” is an amount of the agent that upon administration along with the active ingredient does not cause any material or detectable change of the endogenous level, e.g., homeostatic level of the agent in vivo. Alternatively an “in vivo stabilizing amount” is an amount of the agent that upon administration along with the active ingredient inhibits or decreases one or more adverse effect(s) of the active ingredient without the agent.

In some embodiments, the in vivo stabilizing amount of the agent is an amount that sufficiently saturates binding sites, e.g., available binding sites of the active ingredient to the agent. For example, the in vivo stabilizing amount of the agent can be an amount that capable of binding or binds to at least 0.001%, 0.1%, 0.5%, 1%, 2%, 5%, 10%, 20%, 30%, 40%, or 50% of binding sites, e.g., available binding sites of the active ingredient to the agent. In some other embodiments, the in vivo stabilizing amount of the agent is an amount that upon administration along with the active ingredient does not materially affect or cause detectable change of the pH (e.g., induces a change less than about 0.5 pH units, 0.2 pH units, 0.1 pH units, etc.) of the local site, tissue, or cell environment, etc.

In yet some other embodiments, the in vivo stabilizing amount of the agent is the amount that upon mixing with the active ingredient produces less than a predetermined level of free agent in the composition, e.g., minimum or undetectable level of free agent in the composition. For example, the predetermined level of free agent in the composition can be at least less than 0.1 mM, 0.5 mM, 1 mM, 1.5 mM, or 2 mM in a composition when the active ingredient is an oligonucleotide decoy and the agent is an ion, e.g., calcium. In another example, the predetermined level of the free agent in the composition is less than about 1%, 2%, 5%, 10%, 20%, 30%, 40%, 50%, 60%, 70%, 80%, 90% or 100% of the endogenous level, e.g., local concentration of the agent. In yet another example, the predetermined level of free agent in the composition is determined based on the saturation level of the binding sites in the active ingredient to the agent.

According to the present invention, the free agent is the agent that is not bound to the active ingredient, e.g., by electrostatic, covalent, or hydrophobic interactions, or any other mode of interaction. Alternatively the free agent is the agent that is capable of interfering or interferes with the endogenous level of the agent, e.g., systematically or at the local site of administration.

In still some other embodiments, the in vivo stabilizing amount of the agent is the amount that provide suitable ratio between the active ingredient and the agent so that when they are administered in vivo, it inhibits or decreases one or more adverse effect(s) of the active ingredient without the agent or alternatively it does not cause substantial or detectable change of endogenous level, e.g., homeostatic level of the agent. In some embodiments, the molar ratio or the weight ratio of the active ingredient to the agent ranges from about 1:1000 to about 1000:1. Non-limiting examples of ratios include 1:1, 1:5, 1:10, 1:50, 1:100, 1:250, 1:500, 1:1000, 1000:1, 500:1, 250:1, 100:1, 50:1, 10:1, 5:1, and any range derivable therein inclusive of fractions of integers (e.g., 100.5, 100.05, etc.). Further non-limiting examples of ratios include 1:1, 1:2, 1:3, 1:4, 1:5, 1:6, 1:7, 1:8, 1:9, 1:10, 10:1, 9:1, 8:1, 7:1, 6:1, 5:1, 4:1, 3:1, and 2:1, and any range derivable therein, inclusive of fractions of integers (e.g., 1.5, 1.05, etc.).

In some embodiments, the active ingredient is a nucleic acid, such as an oligonucleotide (e.g., an oligonucleotide decoy), and the agent is a calcium ion, and wherein the weight ratio or the molar ratio of the active ingredient and the agent is from about 0.005 to 5, 0.05 to 5, 0.1 to 3, 0.2 to 2.8, 0.5 to 2, or 1 to 2.

In some embodiments, the active ingredient is a nucleic acid, such as an oligonucleotide (e.g., an oligonucleotide decoy), and the agent is a calcium ion, and wherein the weight ratio or the molar ratio of the active ingredient and the agent is from about 1 to 0.001, 1 to 0.005, 1 to 0.01, 1 to 0.015, 1 to 0.018, 1 to 0.019, 1 to 0.02, 1 to 0.025, 1 to 0.03, 1 to 0.035, 1 to 0.4, or 1 to 0.5. For example, the weight ratio may be 1:1, 2:1, 4:1, 5:1, 15:1, 30:1, 50:1, 100:1, 200:1, 250:1, 300:1, 400:1, 500:1, or 1000:1.

An agent, such as an ion (e.g., a calcium ion), can be comprised in a composition such as a salt (e.g., CaCl₂), and the molar amount or weight amount of that composition can be referenced in a ratio. Accordingly, in some embodiments, the agent is a calcium ion comprised in a composition such as CaCl₂, wherein the weight ratio of an active ingredient, such as a nucleic acid (e.g., an oligonucleotide, an oligonucleotide decoy) to the composition, e.g., CaCl₂, is about 1:1, 2:1, 4:1, 5:1, 15:1, 30:1, 50:1, 100:1, 200:1, 250:1, 300:1, 400:1, or 500:1, or any range derivable therein.

It is understood that the exact ratio of active ingredient to agent in a composition may vary, such as based on the chemical nature of the active ingredient (e.g., in the context of a nucleic acid, whether the nucleic acid is RNA, DNA, single stranded or double stranded, the percent GC content, or molecular weight), the agent and its local concentration (e.g., endogenous level) in the targeted in vivo site, and its intended delivery route. For example, in an environment with a higher endogenous calcium concentration, it is anticipated that the ratio of active ingredient (e.g., oligonucleotide decoy):calcium should be increased in a composition comprising such components.

In still yet some other embodiments, the in vivo stabilizing amount of the agent is the amount that when administered along with the active ingredient causes minimum, insubstantial, or undetectable amount of interaction, e.g., binding between the endogenous agent and the active ingredient. In some aspects, the formulations present in U.S. patent application Ser. No. 14/399,235 (incorporated herein by reference) are utilized herein.

### Oligonucleotide Decoys

The present invention relates to oligonucleotide decoys, pharmaceutical compositions thereof, and use of such oligonucleotide decoys and pharmaceutical compositions to modulate nociceptive signaling and to prevent and/or treat pain. For example, an oligonucleotide decoy, such as described in U.S. Pat. Nos. 7,943,591; 8,093,225; 8,741,864, and U.S. application Ser. Nos. 14/258,927 and 15/019,791. An “oligonucleotide decoy” refers to any double-stranded, nucleic acid-containing polymer generally less than approximately 200 nucleotides (or 100 base pairs) and including, but not limited to, DNA, RNA and RNA-DNA hybrids. The term encompasses sequences that include any of the known base analogs of DNA and RNA including, but not limited to, 2,6-diaminopurine, 5-carboxymethylaminomethyl-2-thiouracil, 5-carboxymethylaminomethyluracil, dihydrouracil, inosine, uracil-5-oxyacetic acid, N6-isopentenyladenine, 1-methyladenine, N-uracil-5-oxyacetic acid methylester, queosine, 2-thiocytosine, 5-bromouracil, methylphosphonate, phosphorodithioate, ormacetal, 3′-thioformacetal, nitroxide backbone, sulfone, sulfamate, morpholino derivatives, locked nucleic acid (LNA) derivatives, or peptide nucleic acid (PNA) derivatives. In some embodiments, the oligonucleotide decoy is composed of two complementary single-stranded oligonucleotides that are annealed together. In other embodiments, the oligonucleotide decoy is composed of one single-stranded oligonucleotide that forms intramolecular base pairs to create a substantially double-stranded structure.

In certain embodiments, the oligonucleotide decoys comprise one or more (e.g., 1, 2, 3, 4, 5, etc.) transcription factor binding sites. In related embodiments, each transcription factor binding site binds to a transcription factor selected from the group consisting of POU1F1, POU2F, POU3F, POU4F1, POU5F1, USF, EGR1, CREB/ATF, AP1, CEBP, SRF, ETS1, MEF2, SP1, RUNX, NFAT, ELK1, ternary complex factors, STAT, GATA1, ELF1, nuclear factor-granulocyte/macrophage a, HNF1, ZFHX3, IRF, TEAD1, TBP, NFY, caccc-box binding factors, KLF4, KLF7, IKZF, MAF, REST, HSF, KCNIP3 and PPAR transcription factors. In certain embodiments, transcription factor binding sites bind to two or more members of a family of closely-related transcription factors. Representative members of such transcription factor families can be selected from the group consisting of POU1F1, POU2F, POU3F, POU4F1, POU5F1, USF, EGR1, CREB/ATF, AP1, CEBP, SRF, ETS1, MEF2, SP1, RUNX, NFAT, ELK1, ternary complex factors, STAT, GATA1, ELF1, nuclear factor-granulocyte/macrophage a, HNF1, ZFHX3, IRF, TEAD1, TBP, NFY, caccc-box binding factors, KLF4, KLF7, IKZF, MAF, REST, HSF, KCNIP3 and PPAR transcription factors. Thus, in certain embodiments, an oligonucleotide decoy that binds to, e.g., EGR1, can also bind to one or more additional family members, e.g., EGR2, EGR3, EGR4.

In certain embodiments, the oligonucleotide decoys comprise two or more (e.g., 2, 3, 4, 5, etc.) transcription factor binding sites. In related embodiments, each transcription factor binding site binds to a transcription factor selected from the group consisting of POU1F1, POU2F, POU3F, POU4F1, POU5F1, USF, EGR1, CREB/ATF, AP1, CEBP, SRF, ETS1, MEF2, SP1, RUNX, NFAT, ELK1, ternary complex factors, STAT, GATA1, ELF1, nuclear factor-granulocyte/macrophage a, HNF1, ZFHX3, IRF, TEAD1, TBP, NFY, caccc-box binding factors, KLF4, KLF7, IKZF, MAF, REST, HSF, KCNIP3 and PPAR transcription factors. In certain embodiments, the relative position of the two or more transcription factor binding sites within the decoy modulates (e.g., increases or decreases) the binding affinity between a target transcription factor (i.e., the transcription factor that a particular binding site is designed to bind to) and its transcription factor binding site, e.g., as compared to the binding affinity between the transcription factor and a decoy having a single transcription factor binding site (e.g., a consensus binding site) specific to the transcription factor. Thus, the relative position of the two transcription factor binding sites within an oligonucleotide decoy of the invention can increase the affinity of the oligonucleotide decoy for a target transcription factor (e.g., for one or more of the transcription factors targeted by the decoy). In certain embodiments, the increase in affinity of the oligonucleotide decoy for a target transcription factor is 1.2 fold or greater (e.g., about 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0 fold, or more). In certain embodiments, the relative position of the two transcription factor binding sites within an oligonucleotide decoy promotes protein-protein interactions between transcription factors bound to the sites, e.g., homodimerization or heterodimerization of the transcription factors. In certain embodiments, such protein-protein interactions between transcription factors stabilize their interactions, e.g., binding, to the oligonucleotide decoy, thereby increasing the binding affinity of the oligonucleotide decoy for one or more of the target transcription factors.

In certain embodiments, a transcription factor that binds to a transcription factor binding site present in an oligonucleotide decoy is a human transcription factor. In other embodiments, the transcription factor that binds to a transcription factor binding site in an oligonucleotide decoy is a non-human, e.g., an avian, mammal (e.g., mouse, rat, dog, cat, horse, cow, etc.), or primate, transcription factor.

In certain embodiments, the transcription factor binding sites of an oligonucleotide decoy each bind to the same transcription factor, e.g., EGR1. In other embodiments, the transcription factor binding sites of an oligonucleotide decoy bind to different transcription factors, e.g., different members of a closely related family of transcription factors (e.g., different members of the EGR1 family) or a combination of transcription factors selected from the group consisting of POU1F1, POU2F, POU3F, POU4F1, POU5F1, USF, EGR1, CREB/ATF, AP1, CEBP, SRF, ETS1, MEF2, SP1, RUNX, NFAT, ELK1, ternary complex factors, STAT, GATA1, ELF1, nuclear factor-granulocyte/macrophage a, HNF1, ZFHX3, IRF, TEAD1, TBP, NFY, caccc-box binding factors, KLF4, KLF7, IKZF, MAF, REST, HSF, KCNIP3 and PPAR transcription factors.

In certain embodiments, the transcription factor binding sites of an oligonucleotide decoy are separated from each other by a linker sequence. Linker sequences can be 1, 2, 3, 4, 5, 6, 7, 8, 9, 10, or more base pairs in length. Typically, linker sequences will be two to five base pairs in length. In other embodiments, the transcription factor binding sites can be immediately adjacent to one another (e.g., no linker sequence is present) or overlapping. In cases where the transcription factor binding sites are overlapping, the transcription factor binding sites can share 1, 2, 3, 4, 5, or more base pairs. Alternatively, one or both of the transcription factor binding sites can be lacking base pairs that otherwise form part of a consensus binding sequence for the transcription factor(s) that bind to the site. In general, however, base pairs that are critical to the binding interaction between a transcription factor binding site and the transcription factors that bind to the site (e.g., base pairs that are essentially invariant in a consensus binding sequence for a particular transcription factor) are not shared or missing when transcription binding sequences are overlapping.

In certain embodiments, oligonucleotide decoys comprise flanking sequences located at each end of the decoy sequence. Flanking sequences can be 1, 2, 3, 4, 5, 6, or more base pairs in length. In general, flanking sequences are two to five base pairs in length. In preferred embodiments, 5′ flanking sequences starts with a G/C base pair and 3′ flanking sequences terminate in a G/C base pair. In preferred embodiments, flanking sequences do not form part of a transcription factor binding site or do not interact with or bind to transcription factors. In other embodiments, flanking sequences form weak interactions with transcription factors bound to an adjacent transcription factor binding site.

In certain embodiments, oligonucleotide decoys are generally at least 10, 11, 12, 13, 14, 15, or more base pairs in length. In related embodiments, oligonucleotide decoys are generally less than 65, 60, 55, 50, or 45 base pairs in length. In some embodiments, oligonucleotide decoys are about 20 to 40 base pairs in length. In other embodiments, oligonucleotide decoys are about 20 to 35, 25 to 40, or 25 to 35 base pairs in length.

In certain embodiments, the oligonucleotide decoys comprise: (a) a sequence selected from the group consisting of SEQ ID NOs.: 1-40, 42, 45 and 47-53; or (b) a sequence having at least 50%, 55%, 60%, 65%, 70%, 75%, 80%, 85%, 88%, 89%, 90%, 91%, 92%, 93%, 94%, 95%, 96%, 97%, 98%, 99%, or 100% identity with a sequence selected from the group consisting of SEQ ID NOs.: 1-40, 42, 45 and 47-53. In related embodiments, the oligonucleotide decoys comprise a sequence having at least 90% identity with a sequence selected from the group consisting of SEQ ID NOs.: 1-39, 42, 45 and 47-52. In other embodiments, the oligonucleotide decoys comprise a sequence having at least 85% identity with a sequence selected from the group consisting of SEQ ID NOs.: 1-17, 19-39, 42, 45 and 47-53. In other embodiments, the oligonucleotide decoys comprise a sequence having at least 80% identity with a sequence selected from the group consisting of SEQ ID NOs.: 1-5, 7-17, 19-39, 42, 45 and 47-53. In other embodiments, the oligonucleotide decoys comprise a sequence having at least 75% identity with a sequence selected from the group consisting of SEQ ID NOs.: 1-4, 7-9, 13, 15-17, 19-23, 26-39, 45, 48, 50, 51 and 53. In other embodiments, the oligonucleotide decoys comprise a sequence having at least 70% identity with a sequence selected from the group consisting of SEQ ID NOs.: 1-3, 7-9, 13, 15-17, 19-23, 26, 28, 30, 32, 34-36, 38-39 and 48. In other embodiments, the oligonucleotide decoys comprise a sequence having at least 65% identity with a sequence selected from the group consisting of SEQ ID NOs.: 2-3, 9, 13, 15-16, 19-23, 26, 28, 30, 32, 34-36, 38 and 39. In other embodiments, the oligonucleotide decoys comprise a sequence having at least 60% identity with a sequence selected from the group consisting of SEQ ID NOs.: 2, 13, 15-16, 21, 23, 26, 30, 32, 34-36, 38 and 39. In still other embodiments, the oligonucleotide decoys comprise a sequence having at least 55% identity with a sequence selected from the group consisting of SEQ ID NOs.: 16, 23, 30, 32, 34, 35, 38 and 39. In still other embodiments, the oligonucleotide decoys comprise a sequence having at least 50% identity with a sequence selected from the group consisting of SEQ ID NOs.: 30, 32, 35, and 38.

In certain embodiments, an oligonucleotide decoy comprises a double-stranded sequence represented by formula (1):

5′-S₁n₂n₃n₄n₅A₆T₇D₈B₉N₁₀d₁₁d₁₂n₁₃n₁₄n₁₅n₁₆n₁₇A₁₈T₁₉D₂₀ . . . B₂₁N₂₂H₂₃H₂₄n₂₅n₂₆n₂₇n₂₈n₂₉n₃₀S₃₁-3′ (1)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “D” can be an A, G, or T nucleotide, “B” can be a C, G, or T nucleotide, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (1) has at least about 70%, 75%, 80%, 85%, 88%, 90%, 91%, 92%, 93%, 94%, 95%, 96%, 97%, 98%, or 99% sequence identity to the nucleotide sequence of SEQ ID NO.: 1. Such oligonucleotide decoys can bind to POU2F1 transcription factor. In certain embodiments, such oligonucleotide decoys can bind to one or more transcription factors closely related to POU2F1 transcription factor, such as POU2F2, POU3F1-2, and POU5F1.

In certain embodiments, an oligonucleotide decoy represented by formula (1) comprises a deletion of one or more (e.g., 1, 2, 3, 4, 5, 6, or 7) nucleotides selected from the group consisting of d₁₁, d₁₂, n₁₃, n₁₄, n₁₅, n₁₆, and n₁₇. In certain embodiments, oligonucleotide decoys comprising a deletion of one or more nucleotides selected from the group consisting of d₁₁, d₁₂, n₁₃, n₁₄, n₁₅, n₁₆, and n₁₇ have at least 70% identity to the nucleotide sequence of SEQ ID NO.: 1.

In certain embodiments, an oligonucleotide decoy comprises a double-stranded sequence represented by formula (2):

5′-S₁n₂n₃n₄n₅n₆Y₇C₈V₉Y₁₀R₁₁N₁₂G₁₃n₁₄n₁₅c₁₆v₁₇y₁₈d₁₉b₂₀ . . . g₂₁y₂₂C₂₃V₂₄Y₂₅R₂₆B₂₇G₂₈R₂₉n₃₀n₃₁n₃₂n₃₃n₃₄n₃₅S₃₆-3′ (2)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “D” can be an A, G, or T nucleotide, “B” can be a C, G, or T nucleotide, “R” can be a G or an A, “V” can be an A, C, or G, “Y” can be a C or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (2) has at least about 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 2. Such oligonucleotide decoys can bind to USF1 transcription factor. In certain embodiments, such oligonucleotide decoys can bind to one or more transcription factors closely related to USF1 transcription factor, such as USF2.

In certain embodiments, an oligonucleotide decoy represented by formula (2) comprises a deletion of one or more (e.g., 1, 2, 3, 4, 5, 6, 7, 8 or 9) nucleotides selected from the group consisting of n₁₄, n₁₅, c₁₆, v₁₇, y₁₈, d₁₉, b₂₀, g₂₁, and y₂₂. In certain embodiments, oligonucleotide decoys comprising a deletion of one or more nucleotides selected from the group consisting of n₁₄, n₁₅, c₁₆, v₁₇, y₁₈, d₁₉, b₂₀, g₂₁, and y₂₂ have at least 60% identity to the nucleotide sequence of SEQ ID NO.: 2.

In certain embodiments, an oligonucleotide decoy comprises a double-stranded sequence represented by formula (3):

5′-S₁n₂n₃W₄W₅G₆S₇G₈K₉R₁₀G₁₁G₁₂M₁₃n₁₄n₁₅n₁₆w₁₇w₁₈w₁₉g₂₀ . . . s₂₁g₂₂K₂₃R₂₄G₂₅G₂₆M₂₇D₂₈n₂₉n₃₀n₃₁n₃₂n₃₃S₃₄-3′ (3)

wherein “A” is an adenine nucleotide, “C” is a cytosine nucleotide, “G” is a guanine nucleotide, “T” is a thymine nucleotide, “S” can be a G or C nucleotide, “N” can be any nucleotide, ‘W’ can be an A or a T, “D” can be an A, G, or T nucleotide, “R” can be a G or an A, “K” can be a T or a G, “M” can be a C or an A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (3) has at least about 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 3. Such oligonucleotide decoys can bind to EGR1 transcription factor. In certain embodiments, such oligonucleotide decoys can bind to one or more transcription factors closely related to EGR1 transcription factor, such as EGR2-4.

In certain embodiments, an oligonucleotide decoy represented by formula (3) comprises a deletion of one or more (e.g., 1, 2, 3, 4, 5, 6, 7, 8 or 9) nucleotides selected from the group consisting of n₁₄, n₁₅, n₁₆, w₁₇, w₁₈, w₁₉, g₂₀, s₂₁, and g₂₂. In certain embodiments, oligonucleotide decoys comprising a deletion of one or more nucleotides selected from the group consisting of n₁₄, n₁₅, n₁₆, w₁₇, w₁₈, w₁₉, g₂₀, s₂₁, and g₂₂ have at least 65% identity to the nucleotide sequence of SEQ ID NO.: 3.

In certain embodiments, an oligonucleotide decoy comprises a double-stranded sequence represented by formula (4):

5′-S₁n₂n₃n₄n₅n₆n₇T₈K₉A₁₀S₁₁S₁₂b₁₃m₁₄n₁₅n₁₆T₁₇K₁₈A₁₉S₂₀ . . . S₂₁B₂₂M₂₃N₂₄n₂₅n₂₆n₂₇n₂₈S₂₉-3′ (4)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “B” can be a C, G or T, “K” can be a T or a G, “M” can be a C or an A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (4) has at least about 75%, 80%, 85%, 88%, 90%, 91%, 92%, 93%, 94%, 95%, 96%, 97%, 98%, or 99% sequence identity to the nucleotide sequence of SEQ ID NO.: 4. Such oligonucleotide decoys can bind to CREB1 transcription factor. In certain embodiments, such oligonucleotide decoys can bind to one or more transcription factors closely related to CREB1 transcription factor, such as CREB3-5 and ATF1-7.

In certain embodiments, an oligonucleotide decoy represented by formula (4) comprises a deletion of one or more (e.g., 1, 2, 3 or 4) nucleotides selected from the group consisting of b₁₃, m₁₄, n₁₅, and n₁₆. In certain embodiments, oligonucleotide decoys comprising a deletion of one or more nucleotides selected from the group consisting of b₁₃, m₁₄, n₁₅, and nib have at least 75% identity to the nucleotide sequence of SEQ ID NO.: 4.

In certain embodiments, an oligonucleotide decoy comprises a double-stranded sequence represented by formula (5):

5′-S₁S₂n₃n₄n₅n₆T₇G₈A₉S₁₀k₁₁n₁₂h₁₃r₁₄r₁₅r₁₆t₁₇G₁₈A₁₉S₂₀ . . . K₂₁N₂₂H₂₃r₂₄r₂₅n₂₆n₂₇n₂₈S₂₉S₃₀-3′ (5)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “R” can be a G or an A, “K” can be a T or a G, “H” can be a C, T or an A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (5) has at least about 80%, 85%, 88%, 90%, 91%, 92%, 93%, 94%, 95%, 96%, 97%, 98%, or 99% sequence identity to the nucleotide sequence of SEQ ID NO.: 5. Such oligonucleotide decoys can bind to AP1/JUN transcription factors. In certain embodiments, such oligonucleotide decoys can bind to one or more transcription factors closely related to AP1/JUN transcription factors, such as AP1/JUN-B, -D and AP1/FOS.

In certain embodiments, an oligonucleotide decoy represented by formula (5) comprises a deletion of one or more (e.g., 1, 2, 3, 4, 5, 6 or 7) nucleotides selected from the group consisting of k₁₁, n₁₂, h₁₃, r₁₄, r₁₅, r₁₆, and t₁₇. In certain embodiments, oligonucleotide decoys comprising a deletion of one or more nucleotides selected from the group consisting of k₁₁, n₁₂, h₁₃, r₁₄, r₁₅, r₁₆, and t₁₇ have at least 80% identity to the nucleotide sequence of SEQ ID NO.: 5.

In certain embodiments, an oligonucleotide decoy comprises a double-stranded sequence represented by formula (6):

5′-S₁n₂n₃n₄n₅w₆w₇w₈G₉A₁₀T₁₁T₁₂K₁₃T₁₄s₁₅s₁₆a₁₇a₁₈k₁₉S₂₀ . . . n₂₁g₂₂A₂₃T₂₄T₂₅K₂₆T₂₇C₂₈S₂₉A₃₀A₃₁K₃₂S₃₃n₃₄n₃₅n₃₆S₃₇-3′ (6)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be A or T, “K” can be a T or a G, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (6) has at least about 85%, 88%, 90%, 91%, 92%, 93%, 94%, 95%, 96%, 97%, 98%, or 99% sequence identity to the nucleotide sequence of SEQ ID NO.: 6. Such oligonucleotide decoys can bind to CEBPA transcription factor. In certain embodiments, such oligonucleotide decoys can bind to one or more transcription factors closely related to CEBPA transcription factor, such as CEBP-B, -D, -E, -G, -Z.

In certain embodiments, an oligonucleotide decoy represented by formula (6) comprises a deletion of one or more (e.g., 1, 2, 3, 4, 5, 6, 7 or 8) nucleotides selected from the group consisting of s₁₅, s₁₆, a₁₇, a₁₈, k₁₉, s₂₀, n₂₁, and g₂₂. In certain embodiments, oligonucleotide decoys comprising a deletion of one or more nucleotides selected from the group consisting of s₁₅, s₁₆, a₁₇, a₁₈, k₁₉, s₂₀, n₂₁, and g₂₂ have at least 85% identity to the nucleotide sequence of SEQ ID NO.: 6.

In certain embodiments, an oligonucleotide decoy comprises a double-stranded sequence represented by formula (7):

5′-S₁n₂n₃n₄n₅n₆g₇g₈a₉t₁₀r₁₁t₁₂C₁₃C₁₄A₁₅T₁₆A₁₇T₁₈T₁₉A₂₀ . . . G₂₁G₂₂a₂₃g₂₄a₂₅t₂₆n₂₇n₂₈n₂₉n₃₀w₃₁w₃₂s₃₃s₃₄-3′ (7)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or T, Y can be a C or T, “R” can be a G or A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (7) has at least about 70%, 75%, 80%, 85%, 88%, 90%, 91%, 92%, 93%, 94%, 95%, 96%, 97%, 98%, or 99% sequence identity to the nucleotide sequence of SEQ ID NO.: 7. Such oligonucleotide decoys can bind to SRF transcription factor. In certain embodiments, such oligonucleotide decoys can bind to one or more transcription factors closely related to SRF transcription factor, such as ELK1.

In certain embodiments, an oligonucleotide decoy represented by formula (7) comprises a deletion of one or more (e.g., 1, 2, 3, 4, 5, 6, 7, 8, 9, 10, 11, 12, 13, 14, 15, 16 or 17) nucleotides selected from the group consisting of g₇, g₈, a₉, t₁₀, r₁₁, t₁₂, a₂₃, g₂₄, a₂₅, t₂₆, n₂₇, n₂₈, n₂₉, n₃₀, w₃₁, w₃₂ and s₃₃. In certain embodiments, oligonucleotide decoys comprising a deletion of one or more nucleotides selected from the group consisting of g₇, g₈, a₉, t₁₀, r₁₁, t₁₂, a₂₃, g₂₄, a₂₅, t₂₆, n₂₇, n₂₈, n₂₉, n₃₀, w₃₁, w₃₂ and s₃₃ have at least 70% identity to the nucleotide sequence of SEQ ID NO.: 7.

In certain embodiments, an oligonucleotide decoy comprises a double-stranded sequence represented by formula (8):

5′-S₁n₂n₃n₄n₅C₆A₇G₈G₉A₁₀d₁₁d₁₂d₁₃d₁₄d₁₅d₁₆d₁₇d₁₈d₁₉T₂₀ . . . C₂₁C₂₂A₂₃T₂₄A₂₅T₂₆T₂₇A₂₈G₂₉n₃₀n₃₁n₃₂n₃₃S₃₄-3′ (8)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “D” can be an A, T or G, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (8) has at least about 70%, 75%, 80%, 85%, 88%, 90%, 91%, 92%, 93%, 94%, 95%, 96%, 97%, 98%, or 99% sequence identity to the nucleotide sequence of SEQ ID NO.: 8. Such oligonucleotide decoys can bind to SRF transcription factor. In certain embodiments, such oligonucleotide decoys can bind to one or more transcription factors closely related to SRF transcription factor, such as ETS1.

In certain embodiments, an oligonucleotide decoy represented by formula (8) comprises a deletion of one or more (e.g., 1, 2, 3, 4, 5, 6, 7, 8 or 9) nucleotides selected from the group consisting of d₁₁, d₁₂, d₁₃, d₁₄, d₁₅, d₁₆, d₁₇, d₁₈ and d₁₉. In certain embodiments, oligonucleotide decoys comprising a deletion of one or more nucleotides selected from the group consisting of d₁₁, d₁₂, d₁₃, d₁₄, d₁₅, d₁₆, d₁₇, d₁₈ and d₁₉ have at least 70% identity to the nucleotide sequence of SEQ ID NO.: 8.

In certain embodiments, an oligonucleotide decoy comprises a double-stranded sequence represented by formula (9):

5′-S₁n₂n₃n₄n₅C₆T₇A₈W₉A₁₀M₁₁W₁₂T₁₃A₁₄A₁₅n₁₆n₁₇n₁₈n₁₉c₂₀ . . . t₂₁A₂₂W₂₃A₂₄A₂₅A₂₆T₂₇A₂₈A₂₉A₃₀A₃₁n₃₂n₃₃n₃₄S₃₅-3′ (9)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or an T, “M” can be a C or an A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (9) has at least about 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 9. Such oligonucleotide decoys can bind to MEF2A transcription factor. In certain embodiments, such oligonucleotide decoys can bind to one or more transcription factors closely related to MEF2A transcription factor, such as MEF2B-C.

In certain embodiments, an oligonucleotide decoy represented by formula (9) comprises a deletion of one or more (e.g., 1, 2, 3, 4, 5 or 6) nucleotides selected from the group consisting of nib, n₁₇, n₁₈, n₁₉, c₂₀ and t₂₁. In certain embodiments, oligonucleotide decoys comprising a deletion of one or more nucleotides selected from the group consisting of n₁₆, n₁₁, n₁₈, n₁₉, c₂₀ and t₂₁ have at least 65% identity to the nucleotide sequence of SEQ ID NO.: 9.

In certain embodiments, an oligonucleotide decoy comprises a double-stranded sequence represented by formula (10):

5′-n₁n₂n₃n₄R₅R₆G₇S₈C₉S₁₀K₁₁r₁₂r₁₃n₁₄n₁₅n₁₆r₁₇r₁₈G₁₉S₂₀ . . . C₂₁K₂₂R₂₃R₂₄N₂₅n₂₆n₂₇n₂₈n₂₉n₃₀-3′ (10)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “K” can be a T or a G, “R” can be a G or an A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (10) has at least about 80%, 85%, 88%, 90%, 91%, 92%, 93%, 94%, 95%, 96%, 97%, 98%, or 99% sequence identity to the nucleotide sequence of SEQ ID NO.: 10. Such oligonucleotide decoys can bind to SP1 transcription factor. In certain embodiments, such oligonucleotide decoys can bind to one or more transcription factors closely related to SP1 transcription factor, such as SP2-8.

In certain embodiments, an oligonucleotide decoy represented by formula (10) comprises a deletion of one or more (e.g., 1, 2, 3, 4, 5, 6 or 7) nucleotides selected from the group consisting of r₁₂, r₁₃, n₁₄, n₁₅, n₁₆, r₁₇, and rig. In certain embodiments, oligonucleotide decoys comprising a deletion of one or more nucleotides selected from the group consisting of n₁₆, n₁₇, n₁₈, n₁₉, c₂₀ and t₂₁ have at least 80% identity to the nucleotide sequence of SEQ ID NO.: 10.

In certain embodiments, an oligonucleotide decoy comprises a double-stranded sequence represented by formula (11):

5′-n₁n₂n₃n₄n₅G₆G₇C₈G₉G₁₀G₁₁G₁₂s₁₃s₁₄s₁₅s₁₆s₁₇s₁₈s₁₉s₂₀ . . . s₂₁s₂₂s₂₃C₂₄G₂₅G₂₆G₂₇C₂₈G₂₉G₃₀T₃₁T₃₂T₃₃A₃₄C₃₅-3′ (11)

wherein “A” is an adenine nucleotide, “C” is a cytosine nucleotide, “G” is a guanine nucleotide, “T” is a thymine nucleotide, “S” can be a G or C nucleotide, “N” can be any nucleotide, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (11) has at least about 80%, 85%, 88%, 90%, 91%, 92%, 93%, 94%, 95%, 96%, 97%, 98%, or 99% sequence identity to the nucleotide sequence of SEQ ID NO.: 11. Such oligonucleotide decoys can bind to SP1 transcription factor. In certain embodiments, such oligonucleotide decoys can bind to one or more transcription factors closely related to SP1 transcription factor, such as SP2-8.

In certain embodiments, an oligonucleotide decoy represented by formula (11) comprises a deletion of one or more (e.g., 1, 2, 3, 4, 5, 6, 7, 8, 9, 10 or 11) nucleotides selected from the group consisting of s₁₃, s₁₄, s₁₅, s₁₆, s₁₇, s₁₈, s₁₉, s₂₀, s₂₁, s₂₂, and s₂₃. In certain embodiments, oligonucleotide decoys comprising a deletion of one or more nucleotides selected from the group consisting of s₁₃, s₁₄, s₁₅, s₁₆, s₁₇, s₁₈, s₁₉, s₂₀, s₂₁, s₂₂, and s₂₃ have at least 80% identity to the nucleotide sequence of SEQ ID NO.: 11.

In certain embodiments, an oligonucleotide decoy comprises a double-stranded sequence represented by formula (12):

5′-S₁n₂n₃n₄n₅W₆G₇Y₈G₉G₁₀t₁₁d₁₂d₁₃d₁₄d₁₅g₁₆W₁₇G₁₈Y₁₉G₂₀ . . . G₂₁T₂₂D₂₃D₂₄D₂₅D₂₆n₂₇n₂₈S₂₉-3′ (12)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, Y can be a C or a T, “D” can be an A, T or a G, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (12) has at least about 80%, 85%, 88%, 90%, 91%, 92%, 93%, 94%, 95%, 96%, 97%, 98%, or 99% sequence identity to the nucleotide sequence of SEQ ID NO.: 12. Such oligonucleotide decoys can bind to RUNX1 transcription factor. In certain embodiments, such oligonucleotide decoys can bind to one or more transcription factors closely related to RUNX1 transcription factor, such as RUNX2-3.

In certain embodiments, an oligonucleotide decoy represented by formula (12) comprises a deletion of one or more (e.g., 1, 2, 3, 4, 5 or 6) nucleotides selected from the group consisting of t₁₁, h₁₂, h₁₃, h₁₄, h₁₅, and g₁₆. In certain embodiments, oligonucleotide decoys comprising a deletion of one or more nucleotides selected from the group consisting of t₁₁, h₁₂, h₁₃, h₁₄, h₁₅, and g₁₆ have at least 80% identity to the nucleotide sequence of SEQ ID NO.: 12.

In certain embodiments, an oligonucleotide decoy comprises a double-stranded sequence represented by formula (13):

5′-S₁n₂n₃n₄n₅T₆T₇G₈G₉G₁₀G₁₁T₁₂C₁₃A₁₄T₁₅A₁₆n₁₇n₁₈n₁₉n₂₀ . . . C₂₁A₂₂C₂₃A₂₄G₂₅G₂₆A₂₇A₂₈C₂₉C₃₀A₃₁C₃₂A₃₃n₃₄n₃₅S₃₆-3′ (13)

wherein “A” is an adenine nucleotide, “C” is a cytosine nucleotide, “G” is a guanine nucleotide, “T” is a thymine nucleotide, “S” can be a G or C nucleotide, “N” can be any nucleotide, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (13) has at least about 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 13. Such oligonucleotide decoys can bind to RUNX1 transcription factor. In certain embodiments, such oligonucleotide decoys can bind to one or more transcription factors closely related to RUNX1 transcription factor, such as RUNX2-3.

In certain embodiments, an oligonucleotide decoy represented by formula (13) comprises a deletion of one or more (e.g., 1, 2, 3 or 4) nucleotides selected from the group consisting of n₁₇, n₁₈, n₁₉ and n₂₀. In certain embodiments, oligonucleotide decoys comprising a deletion of one or more nucleotides selected from the group consisting of n₁₇, n₁₈, n₁₉ and n₂₀ have at least 60% identity to the nucleotide sequence of SEQ ID NO.: 13.

In certain embodiments, an oligonucleotide decoy comprises a double-stranded sequence represented by formula (14):

5′-S₁n₂n₃n₄n₅n₆C₇H₈G₉G₁₀A₁₁H₁₂R₁₃y₁₄n₁₅n₁₆n₁₇c₁₈C₁₉G₂₀ . . . G₂₁A₂₂H₂₃R₂₄Y₂₅n₂₆n₂₇n₂₈n₂₉n₃₀n₃₁S₃₂-3′ (14)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “R” can be G or A, “H” can be A, T or C, “Y” can be a C or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (14) has at least about 80%, 85%, 88%, 90%, 91%, 92%, 93%, 94%, 95%, 96%, 97%, 98%, or 99% sequence identity to the nucleotide sequence of SEQ ID NO.: 14. Such oligonucleotide decoys can bind to ETS1 transcription factor. In certain embodiments, such oligonucleotide decoys can bind to one or more transcription factors closely related to ETS1 transcription factor, such as ELK1.

In certain embodiments, an oligonucleotide decoy represented by formula (14) comprises a deletion of one or more (e.g., 1, 2, 3, 4 or 5) nucleotides selected from the group consisting of y₁₄, n₁₅, n₁₆, n₁₇ and c₁₈. In certain embodiments, oligonucleotide decoys comprising a deletion of one or more nucleotides selected from the group consisting of y₁₄, n₁₅, n₁₆, n₁₇ and c₁₈ have at least 80% identity to the nucleotide sequence of SEQ ID NO.: 14.

In certain embodiments, an oligonucleotide decoy comprises a double-stranded sequence represented by formula (15):

5′-S₁n₂n₃M₄W₅W₆G₇G₈A₉A₁₀A₁₁A₁₂n₁₃n₁₄d₁₅w₁₆w₁₇g₁₈g₁₉a₂₀ . . . a₂₁a₂₂a₂₃n₂₄n₂₅d₂₆W₂₇G₂₈G₂₉A₃₀A₃₁A₃₂A₃₃n₃₄n₃₅n₃₆n₃₇n₃₈n₃₉S₄₀-3′ (15)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “D” can be an A,G or a T, “W” can be an A or a T, “M” can be C or A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (15) has at least about 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 15. Such oligonucleotide decoys can bind to NFATC1 transcription factor. In certain embodiments, such oligonucleotide decoys can bind to one or more transcription factors closely related to NFATC1 transcription factor, such as NFATC2-4.

In certain embodiments, an oligonucleotide decoy represented by formula (15) comprises a deletion of one or more (e.g., 1, 2, 3, 4, 5, 6, 7, 8, 9, 10, 11, 12, 13, 14 or 15) nucleotides selected from the group consisting of n₁₃, n₁₄, d₁₅, w₁₆, w₁₇, g₁₈, g₁₉, a₂₀, a₂₁, a₂₂, a₂₃, n₂₄, n₂₅, d₂₆ and w₂₇. In certain embodiments, oligonucleotide decoys comprising a deletion of one or more nucleotides selected from the group consisting of n₁₃, n₁₄, d₁₅, w₁₆, w₁₇, g₁₈, g₁₉, a₂₀, a₂₁, a₂₂, a₂₃, n₂₄, n₂₅, d₂₆ and w₂₇ have at least 60% identity to the nucleotide sequence of SEQ ID NO.: 15.

In certain embodiments, an oligonucleotide decoy comprises a double-stranded sequence represented by formula (16):

5′-S₁n₂n₃n₄n₅n₆C₇A₈C₉T₁₀T₁₁C₁₂C₁₃y₁₄v₁₅m₁₆n₁₇n₁₈n₁₉y₂₀ . . . v₂₁C₂₂T₂₃T₂₄C₂₅C₂₆T₂₇G₂₈C₂₉n₃₀n₃₁n₃₂S₃₃-3′ (16)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “V” can be G, A or C, “M” can be C or A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (16) has at least about 55%, 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 16. Such oligonucleotide decoys can bind to ELK1 transcription factor. In certain embodiments, such oligonucleotide decoys can bind to one or more transcription factors closely related to ELK1 transcription factor, such as ETS1.

In certain embodiments, an oligonucleotide decoy represented by formula (16) comprises a deletion of one or more (e.g., 1, 2, 3, 4, 5, 6, 7 or 8) nucleotides selected from the group consisting of y₁₄, v₁₅, m₁₆, n₁₇, n₁₈, n₁₉, y₂₀ and v₂₁. In certain embodiments, oligonucleotide decoys comprising a deletion of one or more nucleotides selected from the group consisting of y₁₄, v₁₅, m₁₆, n₁₇, n₁₈, n₁₉, y₂₀ and v₂₁ have at least 55% identity to the nucleotide sequence of SEQ ID NO.: 16.

In certain embodiments, an oligonucleotide decoy comprises a double-stranded sequence represented by formula (17):

5′-S₁n₂n₃n₄n₅n₆C₇T₈A₉T₁₀A₁₁A₁₂A₁₃T₁₄g₁₅g₁₆c₁₇c₁₈t₁₉A₂₀ . . . T₂₁A₂₂A₂₃A₂₄T₂₅G₂₆g₂₇g₂₈g₂₉g₃₀g₃₁g₃₂g₃₃-3′ (17)

wherein “A” is an adenine nucleotide, “C” is a cytosine nucleotide, “G” is a guanine nucleotide, “T” is a thymine nucleotide, “S” can be a G or C nucleotide, “N” can be any nucleotide, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (17) has at least about 70%, 75%, 80%, 85%, 88%, 90%, 91%, 92%, 93%, 94%, 95%, 96%, 97%, 98%, or 99% sequence identity to the nucleotide sequence of SEQ ID NO.: 17. Such oligonucleotide decoys can bind to ternary complex factors. In certain embodiments, such oligonucleotide decoys can bind to one or more transcription factors closely related to ternary complex factors, such as SRF.

In certain embodiments, an oligonucleotide decoy represented by formula (17) comprises a deletion of one or more (e.g., 1, 2, 3, 4 or 5) nucleotides selected from the group consisting of g₁₅, g₁₆, c₁₇, c₁₈ and t₁₉. In certain embodiments, oligonucleotide decoys comprising a deletion of one or more nucleotides selected from the group consisting of g₁₅, g₁₆, c₁₇, c₁₈ and t₁₉ have at least 70% identity to the nucleotide sequence of SEQ ID NO.: 17.

In certain embodiments, an oligonucleotide decoy comprises a double-stranded sequence represented by formula (18):

5′-S₁n₂n₃n₄n₅n₆n₇W₈W₉C₁₀G₁₁C₁₂G₁₃G₁₄w₁₅w₁₆g₁₇g₁₈w₁₉w₂₀ . . . w₂₁C₂₂C₂₃G₂₄G₂₅W₂₆W₂₇n₂₈n₂₉n₃₀n₃₁n₃₂S₃₃-3′ (18)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can an A or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (18) has at least about 90%, 91%, 92%, 93%, 94%, 95%, 96%, 97%, 98%, or 99% sequence identity to the nucleotide sequence of SEQ ID NO.: 18. Such oligonucleotide decoys can bind to STAT1 transcription factor. In certain embodiments, such oligonucleotide decoys can bind to one or more transcription factors closely related to STAT1 transcription factor, such as STAT2-6.

In certain embodiments, an oligonucleotide decoy represented by formula (18) comprises a deletion of one or more (e.g., 1, 2, 3, 4, 5, 6 or 7) nucleotides selected from the group consisting of w₁₅, w₁₆, g₁₇, g₁₈, w₁₉, w₂₀ and w₂₁. In certain embodiments, oligonucleotide decoys comprising a deletion of one or more nucleotides selected from the group consisting of w₁₅, w₁₆, g₁₇, g₁₈, w₁₉, w₂₀ and w₂₁ have at least 90% identity to the nucleotide sequence of SEQ ID NO.: 18.

In certain embodiments, an oligonucleotide decoy comprises a double-stranded sequence represented by formula (19):

5′-S₁n₂n₃n₄T₅G₆C₇C₈T₉T₁₀A₁₁T₁₂C₁₃T₁₄c₁₅t₁₆n₁₇n₁₈g₁₉g₂₀ . . . G₂₁A₂₂T₂₃A₂₄A₂₅S₂₆n₂₇n₂₈n₂₉n₃₀S₃₁-3′ (19)

wherein “A” is an adenine nucleotide, “C” is a cytosine nucleotide, “G” is a guanine nucleotide, “T” is a thymine nucleotide, “S” can be a G or C nucleotide, “N” can be any nucleotide, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (19) has at least about 65%, 70%, 75%, 80%, 85%, 90%, 91%, 92%, 93%, 94%, 95%, 96%, 97%, 98%, or 99% sequence identity to the nucleotide sequence of SEQ ID NO.: 19. Such oligonucleotide decoys can bind to GATA1 transcription factor. In certain embodiments, such oligonucleotide decoys can bind to one or more transcription factors closely related to GATA1 transcription factor, such as GATA2-4.

In certain embodiments, an oligonucleotide decoy represented by formula (19) comprises a deletion of one or more (e.g., 1, 2, 3, 4, 5 or 6) nucleotides selected from the group consisting of c₁₅, t₁₆, n₁₇, n₁₈, g₁₉ and g₂₀. In certain embodiments, oligonucleotide decoys comprising a deletion of one or more nucleotides selected from the group consisting of c₁₅, t₁₆, n₁₇, n₁₈, g₁₉ and g₂₀ have at least 65% identity to the nucleotide sequence of SEQ ID NO.: 19.

In certain embodiments, an oligonucleotide decoy comprises a double-stranded sequence represented by formula (20):

5′-S₁n₂n₃n₄n₅n₆T₇G₈A₉A₁₀T₁₁w₁₂w₁₃g₁₄a₁₅g₁₆g₁₇a₁₈a₁₉a₂₀ . . . a₂₁w₂₂w₂₃G₂₄C₂₅A₂₆T₂₇G₂₈C₂₉n₃₀n₃₁S₃₂-3′ (20)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can an A or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (20) has at least about 65%, 70%, 75%, 80%, 85%, 90%, 91%, 92%, 93%, 94%, 95%, 96%, 97%, 98%, or 99% sequence identity to the nucleotide sequence of SEQ ID NO.: 20. Such oligonucleotide decoys can bind to ELF1 transcription factor. In certain embodiments, such oligonucleotide decoys can bind to one or more transcription factors closely related to ELF1 transcription factor, such as POU1F1.

In certain embodiments, an oligonucleotide decoy represented by formula (20) comprises a deletion of one or more (e.g., 1, 2, 3, 4, 5, 6, 7, 8, 9, 10, 11 or 12) nucleotides selected from the group consisting of w₁₂, w₁₃, g₁₄, a₁₅, g₁₆, g₁₇, a₁₈, a₁₉, a₂₀, a₂₁, w₂₂ and w₂₃. In certain embodiments, oligonucleotide decoys comprising a deletion of one or more nucleotides selected from the group consisting of w₁₂, w₁₃, g₁₄, a₁₅, g₁₆, g₁₇, a₁₈, a₁₉, a₂₀, a₂₁, w₂₂ and w₂₃ have at least 65% identity to the nucleotide sequence of SEQ ID NO.: 20

In certain embodiments, an oligonucleotide decoy comprises a double-stranded sequence represented by formula (21):

5′-S₁n₂n₃n₄n₅G₆A₇G₈A₉T₁₀T₁₁k₁₂c₁₃a₁₄c₁₅n₁₆n₁₇n₁₈g₁₉a₂₀ . . . g₂₁a₂₂t₂₃T₂₄K₂₅C₂₆A₂₇C₂₈n₂₉n₃₀n₃₁n₃₂S₃₃-3′ (21)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “K” can be a G or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (21) has at least about 60%, 65%, 70%, 75%, 80%, 85%, 90%, 91%, 92%, 93%, 94%, 95%, 96%, 97%, 98%, or 99% sequence identity to the nucleotide sequence of SEQ ID NO.: 21. Such oligonucleotide decoys can bind to “nuclear factor-granulocyte/macrophage a” transcription factors. In certain embodiments, such oligonucleotide decoys can bind to one or more transcription factors closely related to “nuclear factor-granulocyte/macrophage a” transcription factors, such as “nuclear factor-granulocyte/macrophage b-c”.

In certain embodiments, an oligonucleotide decoy represented by formula (21) comprises a deletion of one or more (e.g., 1, 2, 3, 4, 5, 6, 7, 8, 9, 10, 11 or 12) nucleotides selected from the group consisting of k₁₂, c₁₃, a₁₄, c₁₅, n₁₆, n₁₇, n₁₈, g₁₉, a₂₀, g₂₁, a₂₂ and t₂₃. In certain embodiments, oligonucleotide decoys comprising a deletion of one or more nucleotides selected from the group consisting of k₁₂, c₁₃, a₁₄, c₁₅, n₁₆, n₁₇, n₁₈, g₁₉, a₂₀, g₂₁, a₂₂ and t₂₃ have at least 60% identity to the nucleotide sequence of SEQ ID NO.: 21.

In certain embodiments, an oligonucleotide decoy comprises a double-stranded sequence represented by formula (22):

5′-S₁n₂n₃n₄n₅K₆C₇M₈T₉W₁₀A₁₁W₁₂t₁₃r₁₄m₁₅w₁₆n₁₇r₁₈m₁₉w₂₀ . . . K₂₁C₂₂M₂₃T₂₄W₂₅A₂₆W₂₇T₂₈n₂₉n₃₀n₃₁S₃₂-3′ (22)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can an A or a T, “K” can be a G or a T, “M” can be an A or a C, “R” can be an A or a G, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (22) has at least about 65%, 70%, 75%, 80%, 85%, 90%, 91%, 92%, 93%, 94%, 95%, 96%, 97%, 98%, or 99% sequence identity to the nucleotide sequence of SEQ ID NO.: 22. Such oligonucleotide decoys can bind to POU4F1 transcription factor. In certain embodiments, such oligonucleotide decoys can bind to one or more transcription factors closely related to POU4F1 transcription factor, such as POU4F2-3.

In certain embodiments, an oligonucleotide decoy represented by formula (22) comprises a deletion of one or more (e.g., 1, 2, 3, 4, 5, 6, 7 or 8) nucleotides selected from the group consisting of t₁₃, r₁₄, m₁₅, w₁₆, n₁₇, r₁₈, m₁₉ and w₂₀. In certain embodiments, oligonucleotide decoys comprising a deletion of one or more nucleotides selected from the group consisting of t₁₃, r₁₄, m₁₅, w₁₆, n₁₇, r₁₈, m₁₉ and w₂₀ have at least 65% identity to the nucleotide sequence of SEQ ID NO.: 22.

In certain embodiments, an oligonucleotide decoy comprises a double-stranded sequence represented by formula (23):

5′-S₁n₂n₃n₄A₅G₆K₇Y₈A₉A₁₀D₁₁N₁₂D₁₃T₁₄h₁₅h₁₆h₁₇n₁₈n₁₉n₂₀ . . . h₂₁h₂₂H₂₃Y₂₄A₂₅A₂₆D₂₇N₂₈D₂₉T₃₀W₃₁V₃₂M₃₃t₃₄g₃₅C₃₆-3′ (23)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “V” can be G, A or C, “K” can be T or G, “D” can be G, A or T, “H” can be A, T or C, “W” can be A or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (23) has at least about 55%, 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 23. Such oligonucleotide decoys can bind to HNF1A transcription factor. In certain embodiments, such oligonucleotide decoys can bind to one or more transcription factors closely related to HNF1A transcription factor, such as HNF1B-C.

In certain embodiments, an oligonucleotide decoy represented by formula (23) comprises a deletion of one or more (e.g., 1, 2, 3, 4, 5, 6, 7 or 8) nucleotides selected from the group consisting of h₁₅, h₁₆, h₁₇, n₁₈, n₁₉, n₂₀, n₂₁ and h₂₂. In certain embodiments, oligonucleotide decoys comprising a deletion of one or more nucleotides selected from the group consisting of h₁₅, h₁₆, h₁₇, n₁₈, n₁₉, n₂₀, h₂₁ and h₂₂ have at least 55% identity to the nucleotide sequence of SEQ ID NO.: 23.

In certain embodiments, an oligonucleotide decoy comprises a double-stranded sequence represented by formula (24):

5′-S₁n₂n₃n₄n₅A₆A₇T₈A₉A₁₀t₁₁n₁₂n₁₃a₁₄t₁₅T₁₆A₁₇T₁₈T₁₉w₂₀ . . . w₂₁n₂₂n₂₃n₂₄S₂₅-3′ (24)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (24) has at least about 80%, 85%, 90%, 91%, 92%, 93%, 94%, 95%, 96%, 97%, 98%, or 99% sequence identity to the nucleotide sequence of SEQ ID NO.: 24. Such oligonucleotide decoys can bind to ZFHX3 transcription factor. In certain embodiments, such oligonucleotide decoys can bind to one or more transcription factors closely related to ZFHX3 transcription factor, such as ZFHX-2, -4.

In certain embodiments, an oligonucleotide decoy represented by formula (24) comprises a deletion of one or more (e.g., 1, 2, 3, 4 or 5) nucleotides selected from the group consisting of t₁₁, n₁₂, n₁₃, a₁₄ and t₁₅. In certain embodiments, oligonucleotide decoys comprising a deletion of one or more nucleotides selected from the group consisting of t₁₁, n₁₂, n₁₃, a₁₄ and t₁₅ have at least 80% identity to the nucleotide sequence of SEQ ID NO.: 24.

In certain embodiments, an oligonucleotide decoy comprises a double-stranded sequence represented by formula (25):

5′-S₁b₂n₃n₄S₅D₆H₇W₈M₉S₁₀H₁₁k₁₂w₁₃w₁₄m₁₅c₁₆s₁₇s₁₈d₁₉h₂₀ . . . w₂₁m₂₂s₂₃h₂₄K₂₅W₂₆W₂₇M₂₈C₂₉S₃₀n₃₁n₃₂n₃₃n₃₄S₃₅-3′ (25)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or T, “D” can be A, G or T, “H” can be A, C or T, “M” can be A or C, “K” can be G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (25) has at least about 80%, 85%, 90%, 91%, 92%, 93%, 94%, 95%, 96%, 97%, 98%, or 99% sequence identity to the nucleotide sequence of SEQ ID NO.: 25. Such oligonucleotide decoys can bind to IRF1 transcription factor. In certain embodiments, such oligonucleotide decoys can bind to one or more transcription factors closely related to IRF1 transcription factor, such as IRF2.

In certain embodiments, an oligonucleotide decoy represented by formula (25) comprises a deletion of one or more (e.g., 1, 2, 3, 4, 5, 6, 7, 8, 9, 10, 11, 12 or 13) nucleotides selected from the group consisting of k₁₂, w₁₃, w₁₄, m₁₅, c₁₆, s₁₇, s₁₈, d₁₉, h₂₀, w₂₁, m₂₂, s₂₃ and h₂₄. In certain embodiments, oligonucleotide decoys comprising a deletion of one or more nucleotides selected from the group consisting of k₁₂, w₁₃, w₁₄, m₁₅, c₁₆, s₁₇, s₁₈, d₁₉, h₂₀, w₂₁, m₂₂, s₂₃ and h₂₄ have at least 80% identity to the nucleotide sequence of SEQ ID NO.: 25.

In certain embodiments, an oligonucleotide decoy comprises a double-stranded sequence represented by formula (26):

5′-S₁n₂n₃n₄y₅k₆g₇y₈k₉G₁₀A₁₁A₁₂y₁₃h₁₄b₁₅b₁₆n₁₇n₁₈n₁₉y₂₀ . . . h₂₁b₂₂b₂₃k₂₄G₂₅A₂₆A₂₇T₂₈A₂₉T₃₀C₃₁n₃₂n₃₃S₃₄-3′ (26)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “V” can be G, A or C, “K” can be T or G, “D” can be G, A or T, “H” can be A, T or G, “B” can be C, G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (26) has at least about 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 26. Such oligonucleotide decoys can bind to TEAD1 transcription factor. In certain embodiments, such oligonucleotide decoys can bind to one or more transcription factors closely related to TEAD1 transcription factor, such as TEAD2-4.

In certain embodiments, an oligonucleotide decoy represented by formula (26) comprises a deletion of one or more (e.g., 1, 2, 3, 4, 5, 6, 7, 8, 9, 10, 11 or 12) nucleotides selected from the group consisting of y₁₃, h₁₄, b₁₅, b₁₆, n₁₇, n₁₈, n₁₉, y₂₀, h₂₁, b₂₂, b₂₃ and k₂₄. In certain embodiments, oligonucleotide decoys comprising a deletion of one or more nucleotides selected from the group consisting of y₁₃, h₁₄, b₁₅, b₁₆, n₁₇, n₁₈, n₁₉, y₂₀, h₂₁, b₂₂, b₂₃ and k₂₄ have at least 60% identity to the nucleotide sequence of SEQ ID NO.: 26.

In certain embodiments, an oligonucleotide decoy comprises a double-stranded sequence represented by formula (27):

5′-S₁n₂n₃n₄T₅A₆T₇A₈W₉w₁₀w₁₁n₁₂n₁₃d₁₄n₁₅t₁₆a₁₇t₁₈A₁₉W₂₀ . . . w₂₁w₂₂n₂₃n₂₄W₂₅W₂₆T₂₇A₂₈A₂₉D₃₀W₃₁n₃₂n₃₃n₃₄n₃₅n₃₆S₃₇-3′ (27)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, “D” can be an A, G or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (27) has at least about 75%, 80%, 85%, 90%, 91%, 92%, 93%, 94%, 95%, 96%, 97%, 98%, or 99% sequence identity to the nucleotide sequence of SEQ ID NO.: 27. Such oligonucleotide decoys can bind to TBP transcription factor. In certain embodiments, such oligonucleotide decoys can bind to one or more transcription factors closely related to TBP transcription factor, such as TBPL1-2.

In certain embodiments, an oligonucleotide decoy represented by formula (27) comprises a deletion of one or more (e.g., 1, 2, 3, 4, 5, 6, 7, 8, 9, 10, 11, 12, 13 or 14) nucleotides selected from the group consisting of w₁₀, w₁₁, n₁₂, n₁₃, d₁₄, n₁₅, t₁₆, air, t₁₈, W₂₁, W₂₂, n₂₃, n₂₄, and W₂₅. In certain embodiments, oligonucleotide decoys comprising a deletion of one or more nucleotides selected from the group consisting of w₁₀, w₁₁, n₁₂, n₁₃, d₁₄, n₁₅, t₁₆, air, t₁₈, w₂₁, w₂₂, n₂₃, n₂₄, and w₂₅ have a t least 75% identity to the nucleotide sequence of SEQ ID NO.: 27.

In certain embodiments, an oligonucleotide decoy comprises a double-stranded sequence represented by formula (28):

5′-S₁n₂n₃n₄T₅A₆T₇A₈A₉W₁₀W₁₁n₁₂n₁₃n₁₄n₁₅w₁₆w₁₇w₁₈A₁₉A₂₀ . . . W₂₁W₂₂k₂₃n₂₄n₂₅n₂₆n₂₇n₂₈S₂₉-3′ (28)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, “K” can be a G or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (28) has at least about 65%, 70%, 75%, 80%, 85%, 90%, 91%, 92%, 93%, 94%, 95%, 96%, 97%, 98%, or 99% sequence identity to the nucleotide sequence of SEQ ID NO.: 28. Such oligonucleotide decoys can bind to TBP transcription factors. In certain embodiments, such oligonucleotide decoys can bind to one or more transcription factors closely related to TBP transcription factors, such as TBPL1-2.

In certain embodiments, an oligonucleotide decoy represented by formula (28) comprises a deletion of one or more (e.g., 1, 2, 3, 4, 5, 6 or 7) nucleotides selected from the group consisting of nit, n₁₃, n₁₄, n₁₅, w₁₆, w₁₇ and w₁₈. In certain embodiments, oligonucleotide decoys comprising a deletion of one or more nucleotides selected from the group consisting of n₁₂, n₁₃, n₁₄, n₁₅, w₁₆, w₁₇ and w₁₈ have at least 65% identity to the nucleotide sequence of SEQ ID NO.: 28.

In certain embodiments, an oligonucleotide decoy comprises a double-stranded sequence represented by formula (29):

5′-N₁n₂n₃C₄T₅G₆M₇K₈Y₉K₁₀K₁₁Y₁₂t₁₃m₁₄b₁₅y₁₆C₁₇A₁₈A₁₉T₂₀ . . . s₂₁d₂₂n₂₃n₂₄n₂₅S₂₆-3′ (29)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “M” can be an A or a C, “K” can be a G or a T, “Y” can be a C or a T, “B” can be a C, G or T, “D” can be an A, G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (29) has at least about 75%, 80%, 85%, 90%, 91%, 92%, 93%, 94%, 95%, 96%, 97%, 98%, or 99% sequence identity to the nucleotide sequence of SEQ ID NO.: 29. Such oligonucleotide decoys can bind to NFYA transcription factor. In certain embodiments, such oligonucleotide decoys can bind to one or more transcription factors closely related to NFYA transcription factor, such as NFYB-C.

In certain embodiments, an oligonucleotide decoy represented by formula (29) comprises a deletion of one or more (e.g., 1, 2, 3 or 4) nucleotides selected from the group consisting of t₁₃, m₁₄, b₁₅ and y₁₆. In certain embodiments, oligonucleotide decoys comprising a deletion of one or more nucleotides selected from the group consisting of t₁₃, m₁₄, b₁₅ and y₁₆ have at least 75% identity to the nucleotide sequence of SEQ ID NO.: 29.

In certain embodiments, an oligonucleotide decoy comprises a double-stranded sequence represented by formula (30):

5′-S₁n₁₂n₁₃T₄C₅T₆C₇Y₈G₉A₁₀T₁₁T₁₂G₁₃G₁₄Y₁₅y₁₆h₁₇y₁₈b₁₉n₂₀ . . . n₂₁n₂₂y₂₃y₂₄h₂₅h₂₆v₂₇G₂₈A₂₉T₃₀T₃₁G₃₂G₃₃Y₃₄T₃₅C₃₆B₃₇Y₃₈n₃₉S₄₀-3′ (30)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “H” can be A, T or C, “B” can be C, G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (30) has at least about 50%, 55%, 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 30. Such oligonucleotide decoys can bind to NFYA transcription factor. In certain embodiments, such oligonucleotide decoys can bind to one or more transcription factors closely related to NFYA transcription factor, such as NFYB-C.

In certain embodiments, an oligonucleotide decoy represented by formula (30) comprises a deletion of one or more (e.g., 1, 2, 3, 4, 5, 6, 7, 8, 9, 10, 11 or 12) nucleotides selected from the group consisting of y₁₆, h₁₇, y₁₈, b₁₉, n₂₀, n₂₁, n₂₂, y₂₃, y₂₄, h₂₅, h₂₆ and v₂₇. In certain embodiments, oligonucleotide decoys comprising a deletion of one or more nucleotides selected from the group consisting of y₁₆, h₁₇, y₁₈, b₁₉, n₂₀, n₂₁, n₂₂, y₂₃, y₂₄, h₂₅, h₂₆ and v₂₇ have at least 50% identity to the nucleotide sequence of SEQ ID NO.: 30.

In certain embodiments, an oligonucleotide decoy comprises a double-stranded sequence represented by formula (31):

5′-S₁n₂n₃C₄A₅C₆C₇C₈s₉a₁₀s₁₁s₁₂s₁₃w₁₄s₁₅s₁₆s₁₇w₁₈C₁₉A₂₀ . . . C₂₁C₂₂C₂₃a₂₄n₂₅n₂₆n₂₇S₂₈-3′ (31)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (31) has at least about 75%, 80%, 85%, 90%, 91%, 92%, 93%, 94%, 95%, 96%, 97%, 98%, or 99% sequence identity to the nucleotide sequence of SEQ ID NO.: 31. Such oligonucleotide decoys can bind to CACCC-box binding factors.

In certain embodiments, an oligonucleotide decoy represented by formula (31) comprises a deletion of one or more (e.g., 1, 2, 3, 4, 5, 6, 7, 8, 9 or 10) nucleotides selected from the group consisting of s₉, a₁₀, s₁₁, s₁₂, s₁₃, w₁₄, s₁₅, s₁₆, s₁₇ and w₁₈. In certain embodiments, oligonucleotide decoys comprising a deletion of one or more nucleotides selected from the group consisting of s₉, a₁₀, s₁₁, s₁₂, s₁₃, w₁₄, s₁₅, s₁₆, s₁₇ and w₁₈ have at least 75% identity to the nucleotide sequence of SEQ ID NO.: 31.

In certain embodiments, an oligonucleotide decoy comprises a double-stranded sequence represented by formula (32):

5′-S₁n₂n₃C₄C₅T₆W₇T₈G₉C₁₀C₁₁T₁₂y₁₃y₁₄y₁₅y₁₆n₁₈n₁₉n₂₀ . . . y₂₁y₂₂y₂₃y₂₄y₂₅G₂₆C₂₇C₂₈T₂₉C₃₀C₃₁T₃₂W₃₃S₃₄n₃₅n₃₆S₃₇-3′ (32)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “W” can be A or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (32) has at least about 50%, 55%, 60%, 65%70%, 75%, 80%, 85%, 88%, 90%, 91%, 92%, 93%, 94%, 95%, 96%, 97%, 98%, or 99% sequence identity to the nucleotide sequence of SEQ ID NO.: 32. Such oligonucleotide decoys can bind to KLF4 transcription factor. In certain embodiments, such oligonucleotide decoys can bind to one or more transcription factors closely related to KLF4 transcription factor, such as KLF-1, -5.

In certain embodiments, an oligonucleotide decoy represented by formula (32) comprises a deletion of one or more (e.g., 1, 2, 3, 4, 5, 6, 7, 8, 9, 10, 11, 12 or 13) nucleotides selected from the group consisting of y₁₃, y₁₄, y₁₅, y₁₆, y₁₇, n₁₈, n₁₉, n₂₀, y₂₁, y₂₂, y₂₃, y₂₄ and y₂₅. In certain embodiments, oligonucleotide decoys comprising a deletion of one or more nucleotides selected from the group consisting of y₁₃, y₁₄, y₁₅, y₁₆, y₁₇, n₁₈, n₁₉, n₂₀, y₂₁, y₂₂, y₂₃, y₂₄ and y₂₅ have at least 50% identity to the nucleotide sequence of SEQ ID NO.: 32.

In certain embodiments, an oligonucleotide decoy comprises a double-stranded sequence represented by formula (33):

5′-S₁n₂n₃n₄W₅W₆W₇G₈G₉G₁₀w₁₁d₁₂g₁₃n₁₄n₁₅w₁₆w₁₇w₁₈G₁₉G₂₀ . . . G₂₁W₂₂D₂₃G₂₄n₂₅n₂₆n₂₇n₂₈S₂₉-3′ (33)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, “D” can be an A, G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (33) has at least about 75%, 80%, 85%, 90%, 91%, 92%, 93%, 94%, 95%, 96%, 97%, 98%, or 99% sequence identity to the nucleotide sequence of SEQ ID NO.: 33. Such oligonucleotide decoys can bind to KLF7 transcription factor. In certain embodiments, such oligonucleotide decoys can bind to one or more transcription factors closely related to KLF7 transcription factor, such as KLF-1, -2, and -5.

In certain embodiments, an oligonucleotide decoy represented by formula (33) comprises a deletion of one or more (e.g., 1, 2, 3, 4, 5, 6, 7 or 8) nucleotides selected from the group consisting of w₁₁, d₁₂, g₁₃, n₁₄, n₁₅, w₁₆, w₁₇ and w₁₈. In certain embodiments, oligonucleotide decoys comprising a deletion of one or more nucleotides selected from the group consisting of w₁₁, d₁₂, go, n₁₄, n₁₅, w₁₆, w₁₇ and w₁₈ have at least 75% identity to the nucleotide sequence of SEQ ID NO.: 33.

In certain embodiments, an oligonucleotide decoy comprises a double-stranded sequence represented by formula (34):

5′-S₁w₂w₃w₄w₅w₆C₇A₈C₉T₁₀C₁₁A₁₂G₁₃C₁₄w₁₅w₁₆w₁₇w₁₈c₁₉g₂₀ . . . g₂₁w₂₂g₂₃w₂₄G₂₅G₂₆G₂₇W₂₈W₂₉g₃₀w₃₁w₃₂w₃₃w₃₄w₃₅S₃₆-3′ (34)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (34) has at least about 55%, 60%, 65%, 70%, 75%, 80%, 85%, 90%, 91%, 92%, 93%, 94%, 95%, 96%, 97%, 98%, or 99% sequence identity to the nucleotide sequence of SEQ ID NO.: 34. Such oligonucleotide decoys can bind to MAFG transcription factor. In certain embodiments, such oligonucleotide decoys can bind to one or more transcription factors closely related to MAFG transcription factor, such as MAF-A, -B, -F, -K.

In certain embodiments, an oligonucleotide decoy represented by formula (34) comprises a deletion of one or more (e.g., 1, 2, 3, 4, 5, 6, 7, 8, 9 or 10) nucleotides selected from the group consisting of w₁₅, w₁₆, w₁₇, w₁₈, c₁₉, g₂₀, g₂₁, w₂₂, g₂₃ and w₂₄. In certain embodiments, oligonucleotide decoys comprising a deletion of one or more nucleotides selected from the group consisting of w₁₅, w₁₆, w₁₇, w₁₈, c₁₉, g₂₀, g₂₁, w₂₂, g₂₃ and w₂₄ have at least 55% identity to the nucleotide sequence of SEQ ID NO.: 34.

In certain embodiments, an oligonucleotide decoy comprises a double-stranded sequence represented by formula (35):

5′-S₁n₂n₃W₄B₅Y₆A₇G₈Y₉A₁₀C₁₁C1₂D₁₃N₁₄R₁₅G₁₆H₁₇S₁₈A₁₉G₂₀ . . . C₂₁N₂₂N₂₃H₂₄n₂₅n₂₆n₂₇W₂₈B₂₉Y₃₀A₃₁G₃₂Y₃₃A₃₄C₃₅C₃₆D₃₇N₃₈R₃₉G₄₀ . . . H₄₁S₄₂A₄₃G₄₄C₄₅N₄₆N₄₇H₄₈n₄₉n₅₀S₅₁-3′ (35)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, Y can be a C or a T, “H” can be an A, T or a C, “R” can be G or A, “D” can be G, A or T, “Y” can be C or T, “B” can be C,G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (35) has at least about 50%, 55%, 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 35. Such oligonucleotide decoys can bind to REST transcription factor.

In certain embodiments, an oligonucleotide decoy represented by formula (35) comprises a deletion of one or more (e.g., 1, 2 or 3) nucleotides selected from the group consisting of n₂₅, n₂₆ and n₂₇. In certain embodiments, oligonucleotide decoys comprising a deletion of one or more nucleotides selected from the group consisting of n₂₅, n₂₆ and n₂₇ have at least 50% identity to the nucleotide sequence of SEQ ID NO.: 35.

In certain embodiments, an oligonucleotide decoy comprises a double-stranded sequence represented by formula (36):

5′-S₁n₂n₃n₄n₅G₆A₇R₈M₉A₁₀W₁₁k₁₂s₁₃a₁₄g₁₅k₁₆n₁₇n₁₈n₁₉n₂₀ . . . g₂₁a₂₂r₂₃m₂₄A₂₅W₂₆K₂₇S₂₈A₂₉G₃₀K₃₁n₃₂n₃₃n₃₄n₃₅S₃₆-3′ (36)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, “M” can be A or C, “R” can be A or G, “K” can be G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (36) has at least about 60%, 65%, 70%, 75%, 80%, 85%, 90%, 91%, 92%, 93%, 94%, 95%, 96%, 97%, 98%, or 99% sequence identity to the nucleotide sequence of SEQ ID NO.: 36. Such oligonucleotide decoys can bind to KCNIP3 transcription factor.

In certain embodiments, an oligonucleotide decoy represented by formula (36) comprises a deletion of one or more (e.g., 1, 2, 3, 4, 5, 6, 7, 8, 9, 10, 11, 12 or 13) nucleotides selected from the group consisting of k₁₂, s₁₃, a₁₄, g₁₅, k₁₆, n₁₇, n₁₈, n₁₉, n₂₀, g₂₁, a₂₂, r₂₃ and m₂₄. In certain embodiments, oligonucleotide decoys comprising a deletion of one or more nucleotides selected from the group consisting of k₁₂, s₁₃, a₁₄, g₁₅, k₁₆, n₁₇, n₁₈, n₁₉, n₂₀, g₂₁, a₂₂, r₂₃ and m₂₄ have at least 60% identity to the nucleotide sequence of SEQ ID NO.: 36.

In certain embodiments, an oligonucleotide decoy comprises a double-stranded sequence represented by formula (37):

5′-S₁n₂n₃n₄n₅G₆A₇R₈G₉C₁₀C₁₁S₁₂s₁₃w₁₄g₁₅w₁₆n₁₇n₁₈n₁₉n₂₀ . . . g₂₁a₂₂r₂₃G₂₄C₂₅C₂₆S₂₇S₂₈W₂₉G₃₀W₃₁n₃₂n₃₃n₃₄S₃₅-3′ (37)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, “M” can be A or C, “R” can be A or G, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (37) has at least about 75%, 80%, 85%, 90%, 91%, 92%, 93%, 94%, 95%, 96%, 97%, 98%, or 99% sequence identity to the nucleotide sequence of SEQ ID NO.: 37. Such oligonucleotide decoys can bind to KCNIP3 transcription factor.

In certain embodiments, an oligonucleotide decoy represented by formula (37) comprises a deletion of one or more (e.g., 1, 2, 3, 4, 5, 6, 7, 8, 9, 10 or 11) nucleotides selected from the group consisting of s₁₃, w₁₄, g₁₅, w₁₆, n₁₇, n₁₈, n₁₉, n₂₀, g₂₁, a₂₂ and r₂₃. In certain embodiments, oligonucleotide decoys comprising a deletion of one or more nucleotides selected from the group consisting of s₁₃, w₁₄, g₁₅, w₁₆, n₁₇, n₁₈, n₁₉, n₂₀, g₂₁, a₂₂ and r₂₃ have at least 75% identity to the nucleotide sequence of SEQ ID NO.: 37.

In certain embodiments, an oligonucleotide decoy comprises a double-stranded sequence represented by formula (38):

5′-s₁C₂G₂A₄A₅A₆G₇G₈A₉C₁₀A₁₁A₁₂A₁₃s₁₄s₁₅n₁₆v₁₇v₁₈n₁₉n₂₀ . . . n₂₁s₂₂g₂₃d₂₄n₂₅n₂₆G₂₇G₂₈A₂₉C₃₀A₃₁A₃₂A₃₃G₃₄G₃₅T₃₆C₃₇A₃₈S₃₉-3′ (38)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “V” can be A, C or G, “D” can be G, A or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (38) has at least about 50%, 55%, 60%, 65%, 70%, 75%, 80%, 85%, 88%, 90%, 91%, 92%, 93%, 94%, 95%, 96%, 97%, 98%, or 99% sequence identity to the nucleotide sequence of SEQ ID NO.: 38. Such oligonucleotide decoys can bind to PPARA transcription factor. In certain embodiments, such oligonucleotide decoys can bind to one or more transcription factors closely related to PPARA transcription factor, such as PPAR-D, -G.

In certain embodiments, an oligonucleotide decoy represented by formula (38) comprises a deletion of one or more (e.g., 1, 2, 3, 4, 5, 6, 7, 8, 9 or 10) nucleotides selected from the group consisting of s₁₄, s₁₅, n₁₆, v₁₇, v₁₈, n₁₉, n₂₀, n₂₁, s₂₂ and g₂₃. In certain embodiments, oligonucleotide decoys comprising a deletion of one or more nucleotides selected from the group consisting of s₁₄, s₁₅, n₁₆, v₁₇, v₁₈, n₁₉, n₂₀, n₂₁, s₂₂ and g₂₃ have at least 50% identity to the nucleotide sequence of SEQ ID NO.: 38.

In certain embodiments, an oligonucleotide decoy comprises a double-stranded sequence represented by formula (39):

5′-S₁n₂n₃n₄A₅R₆M₇R₈W₉W₁₀y₁₁w₁₂m₁₃g₁₄n₁₅n₁₆a₁₇r₁₈m₁₉r₂₀ . . . w₂₁w₂₂y₂₃W₂₄M₂₅G₂₆A₂₇A₂₈T₂₉T₃₀n₃₁n₃₂n₃₃n₃₄S₃₅-3′ (39)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, “R” can be A or G, “M” can be an A or a C, “Y” can be a C or a T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (39) has at least about 55%, 60%, 65%, 70%, 75%, 80%, 85%, 90%, 91%, 92%, 93%, 94%, 95%, 96%, 97%, 98%, or 99% sequence identity to the nucleotide sequence of SEQ ID NO.: 39. Such oligonucleotide decoys can bind to HSF1 transcription factor. In certain embodiments, the oligonucleotide decoys can bind to one or more transcription factors closely related to HSF1 transcription factor, such as HSF2.

In certain embodiments, an oligonucleotide decoy represented by formula (39) comprises a deletion of one or more (e.g., 1, 2, 3, 4, 5, 6, 7, 8, 9, 10, 11, 12 or 13) nucleotides selected from the group consisting of y₁₁, w₁₂, m₁₃, g₁₄, n₁₅, n₁₆, a₁₇, r₁₈, m₁₉, r₂₀, w₂₁, w₂₂ and y₂₃. In certain embodiments, oligonucleotide decoys comprising a deletion of one or more nucleotides selected from the group consisting of y₁₁, w₁₂, m₁₃, g₁₄, n₁₅, n₁₆, a₁₇, r₁₈, m₁₉, r₂₀, w₂₁, w₂₂ and y₂₃ have at least 55% identity to the nucleotide sequence of SEQ ID NO.: 39.

In certain embodiments, an oligonucleotide decoy comprises a double-stranded sequence represented by formula (47):

5′-S₁n₂n₃n₄n₅n₆C₇A₈C₉T₁₀T₁₁C₁₂C₁₃T₁₄G₁₅C₁₆n₁₇n₁₈n₁₉n₂₀n₂₁S₂₂- 3′ (47)

wherein “A” is an adenine nucleotide, “C” is a cytosine nucleotide, “G” is a guanine nucleotide, “T” is a thymine nucleotide, “S” can be a G or C nucleotide, “N” can be any nucleotide, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (47) has at least about 80%, 85%, 88%, 90%, 91%, 92%, 93%, 94%, 95%, 96%, 97%, 98%, or 99% sequence identity to the nucleotide sequence of SEQ ID NO.: 47. Such oligonucleotide decoys can bind to ELK1 transcription factor. In certain embodiments, such oligonucleotide decoys can bind to one or more transcription factors closely related to ELK1 transcription factor, such as ETS1.

In certain embodiments, an oligonucleotide decoy represented by formula (47) comprises a deletion of one or more (e.g., 1, 2, 3, 4, 5, 6, 7, 8, 9 or 10) nucleotides selected from the group consisting of n₂, n₃, n₄, n₅, n₆, n₁₇, n₁₈, n₁₉, n₂₀ and n₂₁. In certain embodiments, oligonucleotide decoys comprising a deletion of one or more nucleotides selected from the group consisting of n₂, n₃, n₄, n₅, n₆, n₁₇, n₁₈, n₁₉, n₂₀ and n₂₁ have at least 80% identity to the nucleotide sequence of SEQ ID NO.: 47.

In certain embodiments, an oligonucleotide decoy comprises a double-stranded sequence represented by formula (48):

5′-S₁n₂n₃n₄n₅n₆A₇G₈K₉Y₁₀A₁₁A₁₂D₁₃N₁₄D₁₅T₁₆W₁₇V₁₈M₁₉N₂₀ . . . n₂₁n₂₂n₂₃n₂₄n₂₅S₂₆-3′ (48)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “V” can be G, A or C, “K” can be T or G, “D” can be G, A or T, “W” can be A or T, “M” can be C or A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (48) has at least about 70%, 75%, 80%, 85%, 88%, 90%, 91%, 92%, 93%, 94%, 95%, 96%, 97%, 98%, or 99% sequence identity to the nucleotide sequence of SEQ ID NO.: 48. Such oligonucleotide decoys can bind to HNF1A transcription factor. In certain embodiments, such oligonucleotide decoys can bind to one or more transcription factors closely related to FINF1A transcription factor, such as FINF1B-C.

In certain embodiments, an oligonucleotide decoy represented by formula (48) comprises a deletion of one or more (e.g., 1, 2, 3, 4, 5, 6, 7, 8, 9 or 10) nucleotides selected from the group consisting of n₂, n₃, n₄, n₅, n₆, n₂₁, n₂₂, n₂₃, n₂₄ and n₂₅. In certain embodiments, oligonucleotide decoys comprising a deletion of one or more nucleotides selected from the group consisting of n₂, n₃, n₄, n₅, n₆, n₂₁, n₂₂, n₂₃, n₂₄ and n₂₅ have at least 70% identity to the nucleotide sequence of SEQ ID NO.: 48.

In certain embodiments, an oligonucleotide decoy comprises a double-stranded sequence represented by formula (49):

5′-S₁n₂n₃T₄C₅T₆C₇Y₈G₉A₁₀T₁₁T₁₂G₁₃G₁₄Y₁₅T₁₆C₁₇B₁₈Y₁₉n₂₀S₂₁-3′ (49)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “B” can be C, G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (49) has at least about 80%, 85%, 88%, 90%, 91%, 92%, 93%, 94%, 95%, 96%, 97%, 98%, or 99% sequence identity to the nucleotide sequence of SEQ ID NO.: 49. Such oligonucleotide decoys can bind to NFYA transcription factor. In certain embodiments, such oligonucleotide decoys can bind to one or more transcription factors closely related to NFYA transcription factor, such as NFYB-C.

In certain embodiments, an oligonucleotide decoy represented by formula (49) comprises a deletion of one or more (e.g., 1, 2 or 3) nucleotides selected from the group consisting of n₂, n₃ and n₂₀. In certain embodiments, oligonucleotide decoys comprising a deletion of one or more nucleotides selected from the group consisting of n₂, n₃ and n₂₀ have at least 80% identity to the nucleotide sequence of SEQ ID NO.: 49.

In certain embodiments, an oligonucleotide decoy comprises a double-stranded sequence represented by formula (50):

5′-S₁n₂n₃n₄n₅n₆C₇C₈T₉W₁₀T₁₁G₁₂C₁₃C₁₄T₁₅C₁₆C₁₇T₁₈W₁₉S₂₀ . . . r₂₁r₂₂n₂₃n₂₄n₂₅S₂₆-3′ (50)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be A or T, “R” can be G or A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (50) has at least about 75%, 80%, 85%, 88%, 90%, 91%, 92%, 93%, 94%, 95%, 96%, 97%, 98%, or 99% sequence identity to the nucleotide sequence of SEQ ID NO.: 50. Such oligonucleotide decoys can bind to KLF4 transcription factor. In certain embodiments, such oligonucleotide decoys can bind to one or more transcription factors closely related to KLF4 transcription factor, such as KLF-1, -5.

In certain embodiments, an oligonucleotide decoy represented by formula (50) comprises a deletion of one or more (e.g., 1, 2, 3, 4, 5, 6, 7, 8, 9 or 10) nucleotides selected from the group consisting of n₂, n₃, n₄, n₅, n₆, r₂₁, r₂₂, n₂₃, n₂₄ and n₂₅. In certain embodiments, oligonucleotide decoys comprising a deletion of one or more nucleotides selected from the group consisting of n₂, n₃, n₄, n₅, n₆, r₂₁, r₂₂, n₂₃, n₂₄ and n₂₅ have at least 75% identity to the nucleotide sequence of SEQ ID NO.: 50.

In certain embodiments, an oligonucleotide decoy comprises a double-stranded sequence represented by formula (51):

5′-S₁n₂n₃n₄n₅W₆B7Y₈A₉G₁₀Y₁₁A₁₂C₁₃C₁₄D₁₅N₁₆R₁₇G₁₈H₁₉S₂₀ . . . A₂₁G₂₂C₂₃N₂₄N₂₅H₂₆n₂₇n₂₈n₂₉n₃₀S₃₁-3′ (51)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be an A or a T, “H” can be an A, T or a C, “R” can be G or A, “D” can be G, A or T, “Y” can be C or T, “B” can be C, G or T, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (51) has at least about 75%, 80%, 85%, 88%, 90%, 91%, 92%, 93%, 94%, 95%, 96%, 97%, 98%, or 99% sequence identity to the nucleotide sequence of SEQ ID NO.: 51. Such oligonucleotide decoys can bind to REST transcription factor.

In certain embodiments, an oligonucleotide decoy represented by formula (51) comprises a deletion of one or more (e.g., 1, 2, 3, 4, 5, 6, 7 or 8) nucleotides selected from the group consisting of n₂, n₃, n₄, n₅, n₂₇, n₂₈, n₂₉ and n₃₀. In certain embodiments, oligonucleotide decoys comprising a deletion of one or more nucleotides selected from the group consisting of n₂, n₃, n₄, n₅, n₂₇, n₂₈, n₂₉ and n₃₀ have at least 75% identity to the nucleotide sequence of SEQ ID NO.: 51.

In certain embodiments, an oligonucleotide decoy comprises a double-stranded sequence represented by formula (52):

5′-S₁m₂r₃m₄W₅A₆G₇G₈N₉C₁₀A₁₁A₁₂A₁₃G₁₄G₁₅T₁₆C₁₇A₁₈n₁₉n₂₀ . . . n₂₁n₂₂S₂₃-3′ (52)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “W” can be A or T, “R” can be G or A, “M” can be C or A, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (52) has at least about 80%, 85%, 88%, 90%, 91%, 92%, 93%, 94%, 95%, 96%, 97%, 98%, or 99% sequence identity to the nucleotide sequence of SEQ ID NO.: 52. Such oligonucleotide decoys can bind to PPARA transcription factor. In certain embodiments, such oligonucleotide decoys can bind to one or more transcription factors closely related to PPARA transcription factor, such as PPAR-D, -G.

In certain embodiments, an oligonucleotide decoy represented by formula (52) comprises a deletion of one or more (e.g., 1, 2, 3, 4, 5, 6, 7 or 8) nucleotides selected from the group consisting of m₂, r₃, m₄, n₁₉, n₂₀, n₂₁, n₂₂ and g₂₃. In certain embodiments, oligonucleotide decoys comprising a deletion of one or more nucleotides selected from the group consisting of m₂, r₃, m₄, n₁₉, n₂₀, n₂₁, n₂₂ and g₂₃ₕ have at least 80% identity to the nucleotide sequence of SEQ ID NO.: 52.

In certain embodiments, an oligonucleotide decoy comprises a double-stranded sequence represented by formula (53):

5′-S₁s₂c₃t₄t₅g₆y₇k₈g₉y₁₀k₁₁G₁₂A₁₃A₁₄T₁₅A₁₆T₁₇c₁₈g₁₉n₂₀ . . . n₂₁n₂₂n₂₃n₂₄S₂₅-3′ (53)

wherein “A” is an adenine nucleotide, “C” is a cytosine nucleotide, “G” is a guanine nucleotide, “T” is a thymine nucleotide, “S” can be a G or C nucleotide, “N” can be any nucleotide, “Y” can be T or C, “K” can be T or G, lower case letters can optionally be deleted, and the numbers in subscript represent the position of a nucleotide in the sequence. Although the formula shows a single strand, it should be understood that a complementary strand is included as part of the structure. In preferred embodiments, an oligonucleotide decoy having a sequence represented by formula (53) has at least about 75%, 80%, 85%, 88%, 90%, 91%, 92%, 93%, 94%, 95%, 96%, 97%, 98%, or 99% sequence identity to the nucleotide sequence of SEQ ID NO.: 53. Such oligonucleotide decoys can bind to TEAD1 transcription factor. In certain embodiments, such oligonucleotide decoys can bind to one or more transcription factors closely related to TEAD1 transcription factor, such as TEAD2-4.

In certain embodiments, an oligonucleotide decoy represented by formula (53) comprises a deletion of one or more (e.g., 1, 2, 3, 4, 5, 6, 7, 8, 9, 10, 11, 12, 13, 14, 15, 16 or 17) nucleotides selected from the group consisting of s₂, c₃, t₄, t₅, g₆, y₇, k₈, g₉, y₁₀, k₁₁, c₁₈, g₁₉, n₂₀, n₂₁, n₂₂, n₂₃ and n₂₄. In certain embodiments, oligonucleotide decoys comprising a deletion of one or more nucleotides selected from the group consisting of s₂, c₃, t₄, t₅, g₆, y₇, k₈, g₉, y₁₀, k₁₁, g₁₈, g₁₉, n₂₀, n₂₁, n₂₂, n₂₃ and n₂₄ have at least 75% identity to the nucleotide sequence of SEQ ID NO.: 53.

A double stranded oligonucleotide having a certain percent (e.g., 65%, 70%, 75%, 80%, 85%, 90%, 95%, or 99%) of sequence identity with another sequence means that, when aligned, that percentage determines the level of correspondence of bases arrangement in comparing the two sequences. This alignment and the percent homology or identity can be determined using any suitable software program known in the art that allows local alignment. The software program should be capable of finding regions of local identity between two sequences without the need to include the entire length of the sequences. In some embodiments, such program includes but is not limited to the EMBOSS Pairwise Alignment Algorithm (available from the European Bioinformatics Institute (EBI)), the ClustalW program (also available from the European Bioinformatics Institute (EBI)), or the BLAST program (BLAST Manual, Altschul et al., Natl Cent. Biotechnol. Inf., Natl Lib. Med. (NCIB NLM NIH), Bethesda, Md., and Altschul et al., (1997) NAR 25:3389 3402).

One skilled in the art will recognize that sequences encompassed herein include those that hybridize under stringent hybridization conditions with an exemplified sequence (e.g., SEQ ID NOs.: 1-42, 45, and 47-53). A nucleic acid is hybridizable to another nucleic acid when a single stranded form of the nucleic acid can anneal to the other single stranded nucleic acid under appropriate conditions of temperature and solution ionic strength. Hybridization conditions are well known in the art. In some embodiments, annealing can occur during a slow decrease of temperature from a denaturizing temperature (e.g., 100° C.) to room temperature in a salt containing solvent (e.g., Tris-EDTA buffer).

Generally, the oligonucleotide decoys disclosed herein may be used to bind and, e.g., thereby inhibit, transcription factors that modulate the expression of genes involved with nociceptive signaling and/or a subject's (e.g., patient's) perception of pain. A oligonucleotide decoy disclosed herein designed to bind to a specific transcription factor has a nucleic acid sequence mimicking the endogenous genomic DNA sequence normally bound by the transcription factor. Accordingly, the oligonucleotide decoys disclosed herein inhibit a necessary step for gene expression. Further, the oligonucleotide decoys disclosed herein may bind to a number of different transcription factors.

### Chemically Modified Oligonucleotide Decoys

The oligonucleotide decoys disclosed herein can be chemically modified by methods well known to the skilled artisan (e.g., incorporation of phosphorothioate, methylphosphonate, phosphorodithioate, phosphoramidates, carbonate, thioether, siloxane, acetamidate or carboxymethyl ester linkages between nucleotides) to prevent degradation by nucleases within cells and extra-cellular fluids (e.g., serum, cerebrospinal fluid). Also, oligonucleotide decoys can be designed that form hairpin and dumbbell structures which also prevent or hinder nuclease degradation. Further, the oligonucleotide decoys can also be inserted as a portion of a larger plasmid capable of episomal maintenance or constitutive replication in the target cell in order to provide longer term, enhanced intracellular exposure to the decoy sequence or reduce its degradation. Accordingly, any chemical modification or structural alteration known in the art to enhance oligonucleotide stability is within the scope of the present disclosure. In some embodiments, the oligonucleotide decoys disclosed herein can be attached, for example, to polyethylene glycol polymers, peptides (e.g., a protein translocation domain) or proteins which improve the therapeutic effect of oligonucleotide decoys. Such modified oligonucleotide decoys can preferentially traverse the cell membrane.

In certain embodiments, the oligonucleotide decoys are provided as salts, hydrates, solvates, or N-oxide derivatives. In certain embodiments, the oligonucleotide decoys are provided in solution (e.g., a saline solution having a physiologic pH) or in lyophilized form. In other embodiments, the oligonucleotide decoys are provided in liposomes.

### Kits

In certain embodiments, one or more oligonucleotide decoys are provided in a kit. In certain embodiments, the kit includes an instruction, e.g., for using said one or more oligonucleotide decoys. In certain embodiments, said instruction describes one or more of the methods of the present invention, e.g., a method for preventing or treating pain, a method of modulating gene expression in a cell, a method for modulating nociceptive signaling in a cell, a method for modulating protein degradation in a cell, etc. In certain embodiments, the oligonucleotide decoys provided in a kit are provided in lyophilized form. In certain related embodiments, a kit that comprises one or more lyophilized oligonucleotide decoys further comprises a solution (e.g., a pharamaceutically acceptable saline solution) that can be used to resuspend said one or more of the oligonucleotide decoys.

In certain embodiments, oligonucleotide decoys include, but are not limited to, sequences presented in Table A. In general, the oligonucleotide decoy is generated by annealing the sequence provided in the table with a complementary sequence. To generate a mismatch double-stranded oligonucleotide, the sequence provided in the table can be annealed to a sequence that is only partially complementary. For example, SEQ ID NO.:43 can be annealed to SEQ ID NO.:46 to produce the mismatched sequence, SEQ ID NO.:43/46.

TABLE A

| Oligonucleotide Sequences (5′-3′) | SEQ ID NO. |
|---|---|
| GGCTTATGCAAATTCGAATGCAAATTTGTCG | SEQ ID NO.: 1 |
| CTAAGCCCACGTGACCATTGGCCAGGTGACCAG | SEQ ID NO.: 2 |
| ATC | |
| GTTATGCGTGGGCGATAATGCGGGGGCGTTATA | SEQ ID NO.: 3 |
| G | |
| GCCTCCCTGAGCTCATTGACGTATCTCGG | SEQ ID NO.: 4 |
| CGAATATGACTGAGAATGACTCAGATTTGC | SEQ ID NO.: 5 |
| GGTTCTATGATTTTGGAATCGGATTGTGCAAAG | SEQ ID NO.: 6 |
| AAGC | |
| GCTTCAGGATGTCCATATTAGGAGATCTTGTTC | SEQ ID NO.: 7 |
| G | |
| GGCCACAGGATGTAGGATGTCCATATTAGGATG | SEQ ID NO.: 8 |
| C | |
| GTTCTCTAAAAATAAAAGGCTAAAAATAAAAGT | SEQ ID NO.: 9 |
| CG | |
| ATTAGGGGCGGGGTCCGGGGCGGGGTATTA | SEQ ID NO.: 10 |
| GTTATGGCGGGGCGGGGCGGGGCCGGGCGGTTT | SEQ ID NO.: 11 |
| AC | |
| GGCAATGTGGTTTTAGTGTGGTTTTACGG | SEQ ID NO.: 12 |
| GCCGTTTGGGGTCATAGAACCACAGGAACCACA | SEQ ID NO.: 13 |
| CGG | |
| CATTGCCCGGAAATGGACCGGATGTAATTTCC | SEQ ID NO.: 14 |
| GTTCTTGGAAAATAAATGGAAAATAGTGGAAAA | SEQ ID NO.: 15 |
| TAAGTCG | |
| CGTTCCCACTTCCTGCGACCACTTCCTGCCGGG | SEQ ID NO.: 16 |
| CTGCACCTATAAATGGCCTATAAATGGGGATGC | SEQ ID NO.: 17 |
| GCTTATTTCGCGGAAGGTTTCCCGGAAGTGGCG | SEQ ID NO.: 18 |
| GCTGTGCCTTATCTCTTTGGGATAACTGGCG | SEQ ID NO.: 19 |
| GCTTAATGAATAAGAGGAAAAATGCATGCTGG | SEQ ID NO.: 20 |
| GTTCTGAGATTGCACGATGAGATTTCACAGTCG | SEQ ID NO.: 21 |
| GTCCCGCATAAATAATGGCATCCTTAATCGCG | SEQ ID NO.: 22 |
| GTGCAGGCAAGAGTAGAGACAGGCAAGAGTAGA | SEQ ID NO.: 23 |
| TGC | |
| CCGCCAATAATTAATTATTAAGGCC | SEQ ID NO.: 24 |
| GCTTCGTTCCATTTCCGGTCTCGGTTTCCCCAT | SEQ ID NO.: 25 |
| TC | |
| GCTGCTGTGGAATATCGACCTGTGGAATATCGT | SEQ ID NO.: 26 |
| G | |
| GCCGTATAAATGTGCTATAAAAGTTTTAAGACC | SEQ ID NO.: 27 |
| GTGC | |
| GCCGTATAAATGTGCTATAAAAGCCGTGC | SEQ ID NO.: 28 |
| ATGCTGCGCTTTTCTCCAATCTGCGG | SEQ ID NO.: 29 |
| CGTTCTCCGATTGGTCACGGACTCTCCGATTGG | SEQ ID NO.: 30 |
| TCACGGC | |
| GCGCACCCCAGCCTGGCTCACCCACGCG | SEQ ID NO.: 31 |
| GATCCTTTGCCTCCTTCGATCCTTTGCCTCCTT | SEQ ID NO.: 32 |
| CAAG | |
| GGTGTTTGGGAGAGCTTTGGGAGGATACG | SEQ ID NO.: 33 |
| GCTAATCACTCAGCATTTCGGTGAGGGAAGTGA | SEQ ID NO.: 34 |
| AAG | |
| CCTTTCAGCACCACGGACAGCGCCAGCTTCAGC | SEQ ID NO.: 35 |
| ACCACGGACAGCGCCTCG | |
| GGATCGAACATGGAGTCAGTGAGAAATCAGGAT | SEQ ID NO.: 36 |
| CGG | |
| GGATCGAAGCCGGAGTCAAGGAGGCCCCTGATC | SEQ ID NO.: 37 |
| GG | |
| CCGAAAGGACAAAGGTCAAGTCGAAAGGACAAA | SEQ ID NO.: 38 |
| GGTCAG | |
| CGGGAGAAAATTCGGGAACGTTCAAGAATTGTC | SEQ ID NO.: 39 |
| GG | |
| GTTATGCGTGGGCGTAGATGCGGGGGCGTTATA | SEQ ID NO.: 40 |
| G | |
| GATGCGTGGGCGTAGG | SEQ ID NO.: 41 |
| GTATGCGTGGGCGGTGGGCGTAG | SEQ ID NO.: 42 |
| GTTATGCGTTTGTAGATGCTTTCGTTATAG | SEQ ID NO.: 43 |
| GTTATGCGTGGGCGATATAG | SEQ ID NO.: 44 |
| GATGCGTGGGCGTTGACGTGGAAAATGC | SEQ ID NO.: 45 |
| CTATTTCGAAACGATCTACATTGGCATAAC | SEQ ID NO.: 46 |
| CGTTCCCACTTCCTGCGACCGG | SEQ ID NO.: 47 |
| GGGTGAAGGCAAGAGTAGAGCGGCGG | SEQ ID NO.: 48 |
| CGTTCTCCGATTGGTCACGCG | SEQ ID NO.: 49 |
| GTACTCCCTTTGCCTCCTTCAACCGG | SEQ ID NO.: 50 |
| CCTTATTCAGCACCACGGACAGCGCCATTCG | SEQ ID NO.: 51 |
| GCGAAAGGACAAAGGTCAGGCGG | SEQ ID NO.: 52 |
| GGCTTGCTGTGGAATATCGATGGTG | SEQ ID NO.: 53 |

Reference will now be made in detail to particular embodiments of the disclosure found in the Examples. These Examples are not intended to limit the disclosure to those particular embodiments. To the contrary, the disclosure is intended to cover alternatives, modifications, and equivalents, as may be included within the spirit and scope of the invention, as defined by the appended claims.

### Examples

### I. A Single Preoperative Dose of AYX1, an EGR1 Decoy, Provides Long-Term Reduction of Postoperative Pain

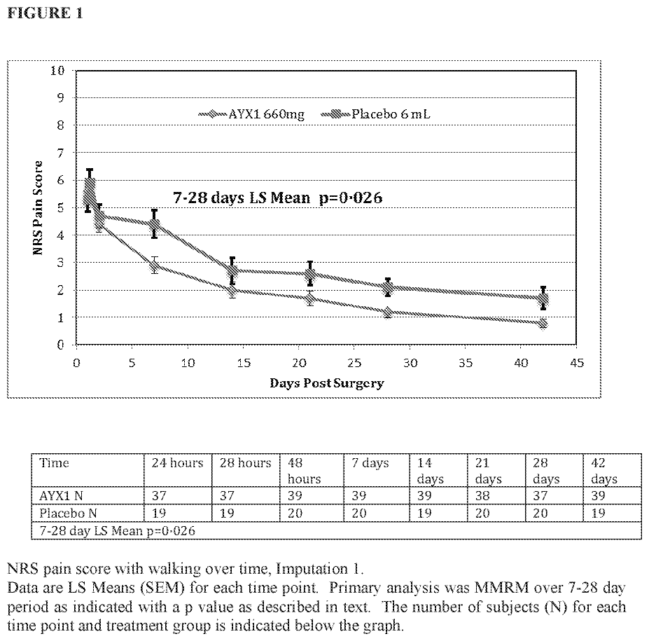

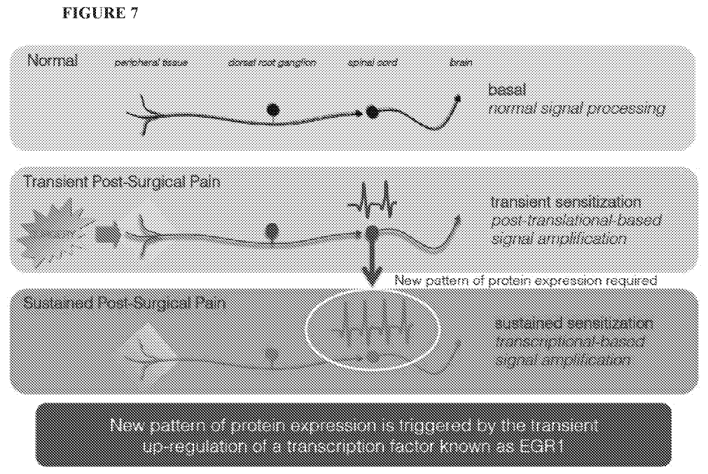

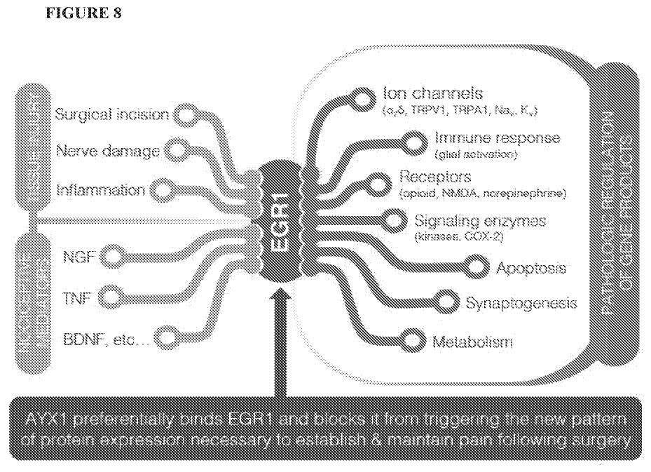

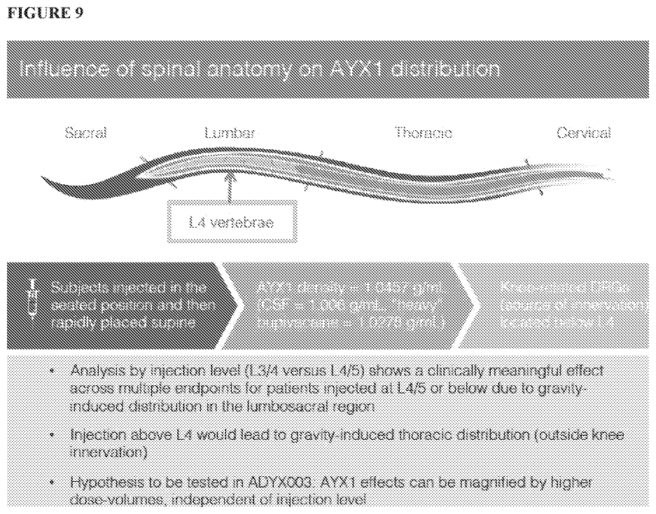

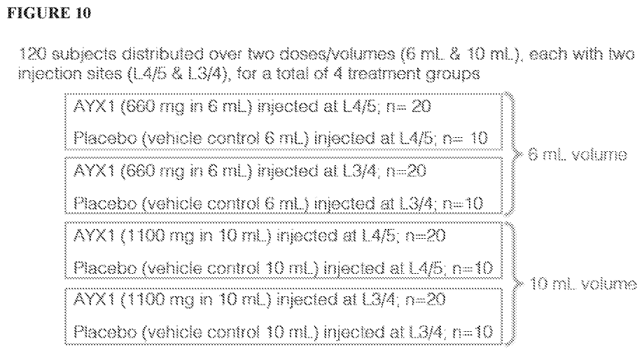

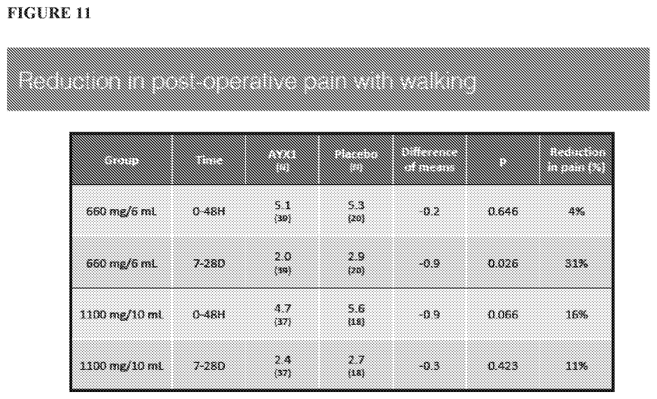

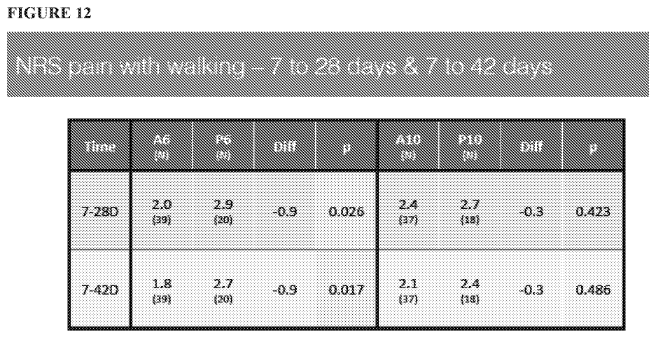

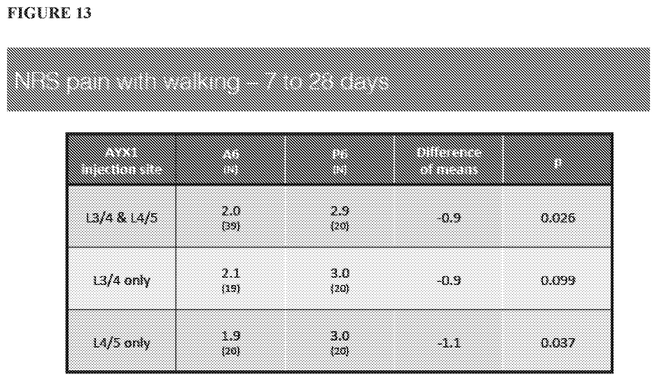

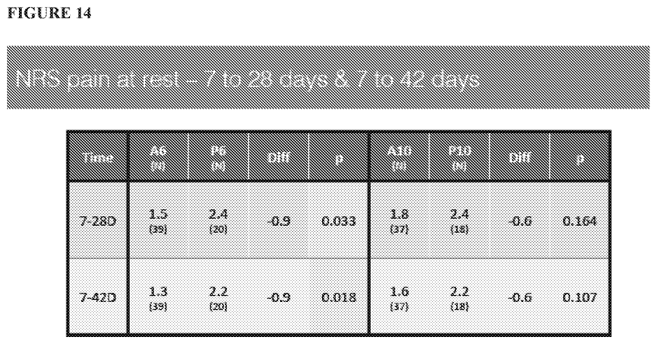

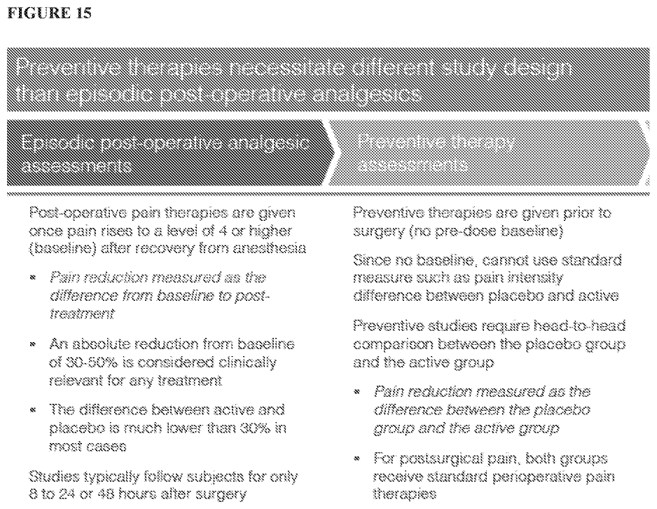

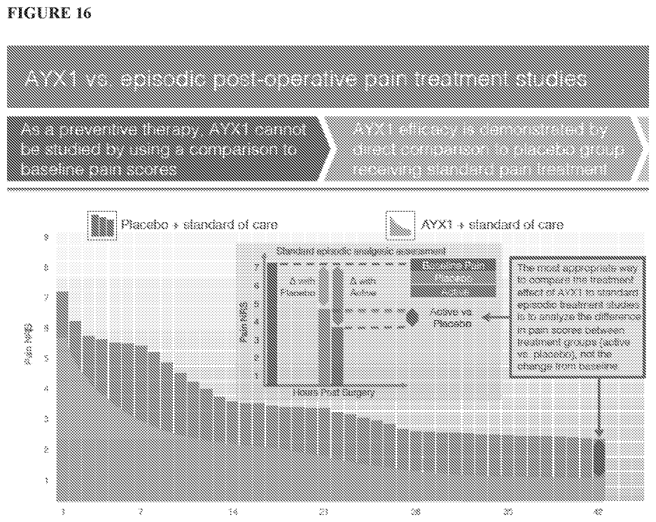

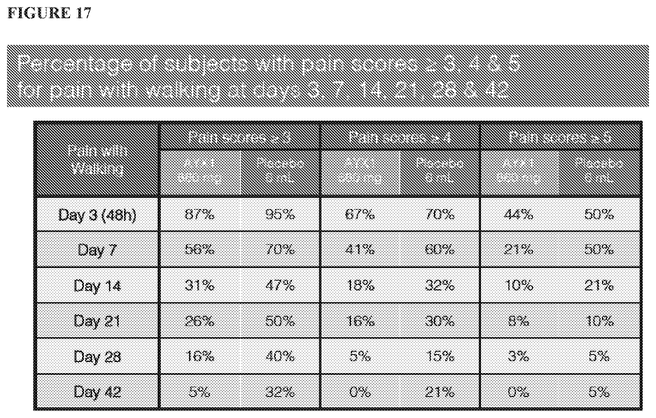

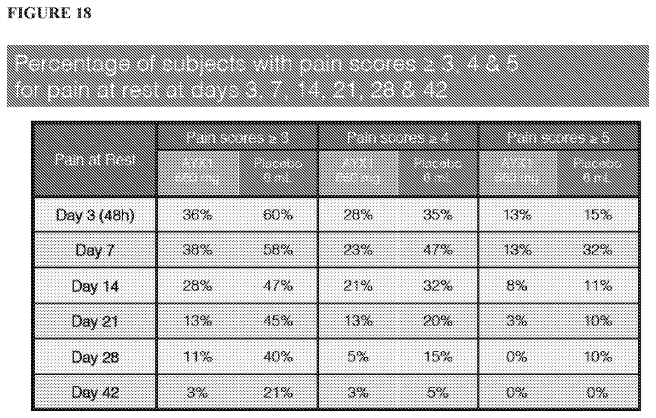

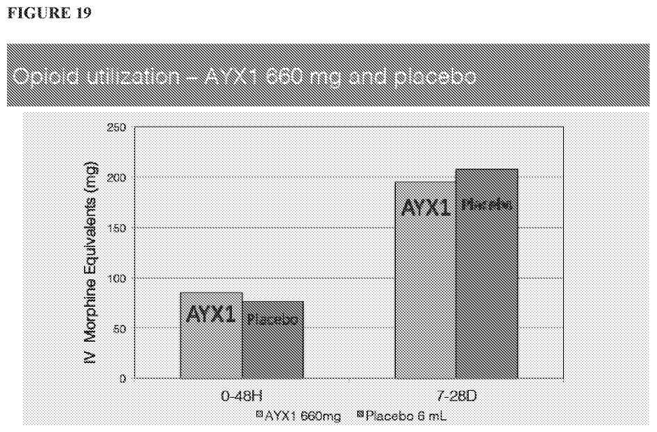

Below, we present a high level overview of the study, which is followed by a detailed study disclosure. Furthermore, FIGS. 1-19 provide additional data and disclosure derived from the below study. As aforementioned, AYX1 is an oligonucleotide decoy, encompassed by SEQ ID NO. 42, with a sense strand of 5′-GTATGCGTGGGCGGTGGGCGTAG-3′ and antisense strand of 3′-CATACGCACCCGCCACCCGCATC-5′.

### Overview of Study

Management of postoperative pain leaves many patients inadequately relieved, burdened with adverse events, and a significant proportion developing chronic pain. We investigated an EGR1 transcription factor decoy, AYX1 (SEQ ID NO. 42), to inhibit the development of postoperative pain following total knee arthroplasty (TKA).

### Methods Overview

This phase 2, randomized, double blind, placebo-controlled study at two U.S. investigational centers enrolled medically stable adults (aged 40-80 years) undergoing TKA with spinal anesthesia, standardized perioperative analgesia with inpatient and outpatient assessments of pain. Subjects were randomly assigned (centrally generated randomization assignments) in a 2:1 ratio to intrathecally receive either AYX1 (660 mg/6 mL or 1100 mg/10 mL) or a volume-matched placebo in four treatment groups at either lumbar interspace L3/4 or L4/5 prior to spinal anesthetic. Study personnel were blinded except for pharmacists and the safety monitoring committee. The primary efficacy endpoints were least square mean pain rating while walking 5 meters (0-48 hours, inpatient period) and 15 meters (7-28 days, outpatient period) as analyzed by a mixed effects model for repeat measures. Safety was assessed in all dosed subjects (safety population) and efficacy was assessed in all dosed subjects with at least one outcome assessment (modified intent-to-treat population). Post-hoc analysis included responder rate (responder defined as NRS<3) at each time point, as well as incidence of pain ≥3 at day 42 for both walking and pain at rest.

Results Overview

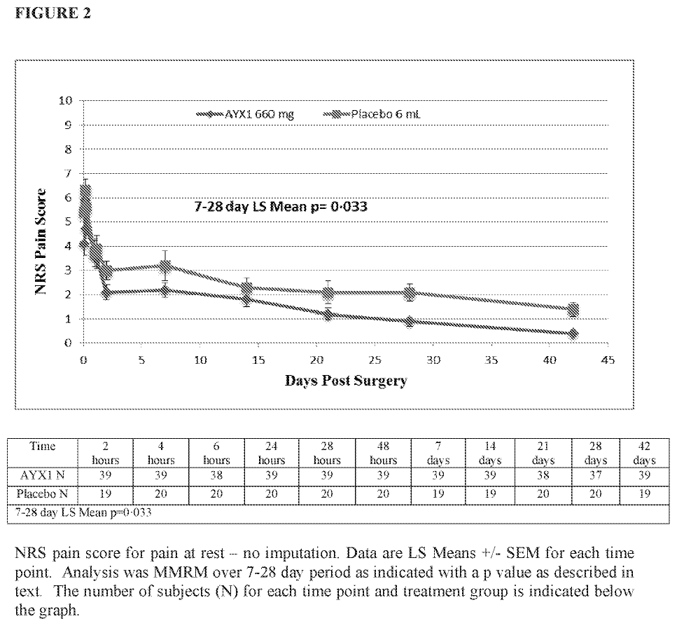

AYX1 660 mg/6 mL plus SOC significantly reduced pain with walking during the outpatient period compared to placebo plus SOC (LS mean 2.0 [SEM 0.2] vs. 2.9 [0.3], p=0.026, FIG. 1); the 1100 mg/10 mL dose did not reach significance (2.4 [0.2] vs. 2.7 [0.4], p=0.423). AYX1 660 mg/6 mL also significantly reduced pain at rest during the outpatient period compared to placebo (1.5 [0.2] vs. 2.4 [0.3] p=0.033, FIG. 2). Neither dose significantly reduced pain from 0-48 hours. Opioid utilization was similar across treatment groups.

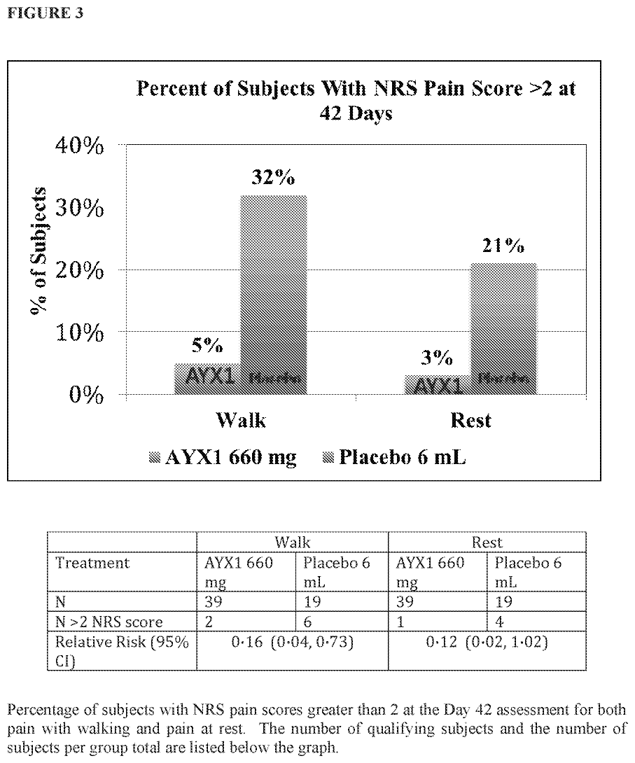

The incidence of pain >2 at day 42 for both walking (5% vs. 32%) and at rest (3% vs. 21%) was markedly reduced in AYX1 660 mg/6 mL-treated subjects compared to placebo (FIG. 3). All AYX1- and all but two placebo-treated subjects reported adverse events. No AYX1-related serious adverse events occurred.

Thus, AYX1 was well tolerated and provided long-term reduction of pain with movement and at rest when added to standard of care. Specifically, a single preoperative intrathecal administration of AYX1 660 mg/6 mL added to a SOC postoperative analgesic regimen was well tolerated and significantly reduced both movement-evoked pain and pain at rest from day 7 through 28, independent of injection site, and the treatment effect persisted through the 42-day follow-up period. The reduced efficacy of the 1100 mg/10 mL dose was consistent with limited, reduced effects at elevated doses/volumes of AYX1 in preclinical pharmacology studies. The proportion of subjects with pain ≥3 at day 42 in the 660 mg/6 mL group strongly support AYX1's ability to prevent postoperative chronic pain.

### Detailed Study

Study in Context

We used published meta-analyses of pharmacologic and regional anesthetic inhibition for prevention of postoperative pain and progression to chronic pain as a guide to the level of effect required for this study.^{29,30} Existing evidence is quite variable given different surgery types and variable application of therapies for preoperative and postoperative periods. Most studies assess average pain or pain at rest up to 48-72 hours after surgery. A review of peripheral nerve blocks adjunctive to systemic analgesia concluded that these nerve blocks provide reduction of pain at rest for all times within 72 hours.²⁹ They also provided reduction of pain with movement but only within 48 to 72 hours. Not enough data was available to make conclusions from the effect of nerve blocks on morphine intake, knee range of motion hospital costs or patient satisfaction. There was also insufficient data to compare peripheral nerve blocks and local infiltration or peripheral nerve blocks and epidural analgesia. Meta-analysis suggested a modest but statistically significant effect of ketamine in reducing chronic pain after surgery³⁰ whereas non-steroidal anti-inflammatory agents and gabapentinoids do not give a reproducible reduction of chronic pain. A search of the literature and ClinTrials.gov did not reveal any studies examining the effect of EGR1 inhibition. An influential paper guiding the study design was Lunn.¹⁴ employing intravenous high dose preoperative methylprednisolone. This study enrolled subjects undergoing TKA with spinal anesthesia on top of standard of care and the primary endpoint was pain with walking at 24 hours post surgery. While methylprednisolone produced a reduction in pain with movement and opioid consumption in the acute postoperative period the effect was not sustained after 48 hours.

Interpretation

This is the first study to show the therapeutic benefit of a preoperative use of a transcription factor decoy to EGR1 in the reduction of acute and potentially the prevention of longer-term and chronic pain. AYX1 reduced movement-evoked pain and pain at rest starting 24-48 hours post surgery and through the entire 42-day study period. This study provided evidence for an optimal dose for AYX1, demonstrated a low AYX1 specific adverse event profile and provided strong surrogate evidence for prevention of the transition to chronic pain. Unlike other therapeutic agents AYX1 had an effect on postoperative pain, especially movement-evoked pain, which outlasted its presence in the CSF and represents true preventive therapy. Other therapeutics may address nociception and potentially short-term sensitization, but it is unclear what effect they have on long-term sensitization. Mechanistically specific agents with a wider influence on pain mediators such as transcription factor decoys appear to be effective beyond standard of care. The unique nature and duration of AYX1 inhibition of postoperative pain beyond standard of care places AYX1 in a distinct class of pain response modifiers.

Introduction

The management of pain following surgery often leaves patients inadequately relieved and burdened with adverse effects.¹⁻⁴ Current approaches focus on blocking one effector (receptor, ion channel, etc.) while leaving many others facilitating nociceptive signaling, thereby only temporarily and partially managing the symptoms of pain. New approaches addressing the initial mechanisms of pain have the potential to reduce acute pain and prevent the transition to chronic pain.

Inhibiting immediate early gene transcription factors that initiate the first waves of gene regulation in response to painful stimuli can alter multiple effectors with long-lasting benefits. One such transcription factor is Early Growth Response 1 (EGR1 also known as zif268 and KROX24), which is transiently induced in dorsal root ganglion (DRG) and spinal cord in response to a wide range of traumatic, inflammatory and neuropathic insults.⁵⁻⁹ Knockout and antisense animal studies revealed that EGR1 is a critical molecular switch converting transient neuronal sensitization into sustained sensitization following a painful stimulus. Without EGR1 early sensitization and pain occur, but are not sustained.^{7,10}

AYX1 is a 23 base-pair, composite, unprotected, double-stranded DNA transcription factor decoy drug candidate designed to inhibit EGR1 activity by binding to and preventing EGR1 interaction with chromosomal DNA.¹¹

We developed AYX1 to inhibit local EGR1 activity in the DRG and spinal cord network in the critical perioperative period to reduce acute postoperative pain and prevent the transition to chronic pain. AYX1 is delivered via a single bolus intrathecal injection immediately prior to surgery and efficiently and dose-dependently prevents the maintenance of mechanical allodynia and hyperalgesia in multiple animal models without altering basal neuronal function.¹¹ Because AYX1 is directed to mechanisms involved in the delayed but persistent long-term sensitization its effects on pain are evident after 24 to 48 hours when sensitization would be fully developed.

AYX1 has been evaluated in two prior clinical studies: a phase 1 dose-escalating safety study (ADYX001) in healthy volunteers¹² and a phase 2a safety and efficacy study (ADYX002) in patients undergoing unilateral TKA.¹³ A total of 79 subjects have received a single intrathecal administration of AYX1 in doses ranging from 1.25 mg to 330 mg without safety concerns related to AYX1.

ADYX002 study protocol, modeled after Lunn,¹⁴ demonstrated that 330 mg AYX1 in 3 mL produced a consistent but non-significant (10%) reduction of pain with walking compared to placebo.¹³ When the data were analyzed as a function of the vertebral interspace of injection, the subjects injected with AYX1 330 mg/3 mL at spinal level L4/5 demonstrated sustained, statistically significant and clinically relevant (˜30%) reductions in movement-evoked pain. We hypothesized that when the subjects were placed supine immediately after injection the limited volume and high specific gravity of AYX1 (10457 g/mL) restricted exposure of AYX1 to the lower lumbar DRG related to the knee. Three mL injections above the L4 peak spinal curve would migrate by gravity to the thoracic region and be ineffective for the knee.

The current study (ADYX003) tested the hypothesis that higher dose/volumes of AYX1 would distribute more widely and have enhanced efficacy independent of the spinal injection site for patients undergoing TKA.

Methods

Study Design and Participants

This was a multicenter, randomized, double blind, placebo-controlled study to evaluate the safety and efficacy of two dose/volume levels of AYX1 administered intrathecally at either L3/4 or L4/5 lumbar sites in patients undergoing primary unilateral total knee arthroplasty (TKA). Two investigative centers (Sheffield, A L and Phoenix, Ariz.) participated involving three community hospitals. Ethics approvals were obtained from a central ethics committee (Western IRB, Puyallup, Wash., USA).

Medically stable males or females, 40-80 years of age, body mass index of 18-40 Kg/m², with vital signs and laboratory values within the acceptable limits for surgery and spinal anesthesia and scheduled to undergo primary unilateral TKA for osteoarthritis were enrolled. Use of opioids in the 30 days prior to randomization was restricted to a daily average of ≤20 mg oral morphine (or equivalent). Preoperative use of adjuvant analgesics (e.g. gabapentinoids, anticonvulsants), benzodiazepines, systemic steroids or nonsteroidal anti-inflammatory drugs (NSAIDs) was prohibited on a schedule detailed in the appendix. General anesthesia, nerve block, local anesthetic infiltration and neuroaxial opioids were prohibited. Several exclusion criteria were intended to minimize the chance of postoperative delirium including sleep apnea. Several screen failures due to sleep apnea led to a protocol amendment primarily to allow subjects with a history of symptomatic sleep apnea without postoperative delirium to be enrolled if current continuous positive airway pressure or bi-level positive airway pressure therapy was continued postoperatively. The complete list of inclusion and exclusion criteria and the rules for concomitant drugs are provided in Table B. Subjects were recruited from participating surgeons' operative lists and signed written informed consent prior to enrollment.

TABLE B
Inclusion Exclusion Criteria

Subjects were required to meet ALL of the following inclusion criteria:

| | |
|---|---|
| 1. | Male or female, between 40-80 years of age, inclusive |
| 2. | Adequately informed of the nature and risks of the study and give written informed |
| | consent before undergoing any study specific assessments or procedures |
| 3. | Scheduled to undergo primary unilateral TKA for painful osteoarthritis without |
| | congenital knee pathology |
| 4. | Have American Society of Anesthesiologists Physical Status Classification System ≤3 |
| 5. | Medically stable as determined by the Investigator based on pre-study medical |
| | history, physical/neurological examination, clinical laboratory tests, and 12-lead |
| | electrocardiogram (ECG) findings |
| 6. | Vital signs and clinical laboratory values within acceptable limits for elective |
| | surgery and spinal anesthesia in the opinion of the Investigator |
| 7. | Body weight ≥34 kg and body mass index of 18-40 kg/m² |
| 8. | Female subjects of child-bearing potential, and those <1 year post-menopausal, |
| | must have a negative serum pregnancy test at screening and agree to practice highly |
| | effective methods of birth control such as hormonal methods (e.g., combined oral, |
| | implantable, injectable, or transdermal contraceptives), other implantable methods |
| | (e.g., intrauterine device), double barrier methods (e.g., condoms, sponge, |
| | diaphragm, or vaginal ring plus spermicidal jellies or cream), or total abstinence |
| | from intercourse for 1 month after study drug administration |
| 9. | Male subjects who are sexually active must agree to use effective barrier |
| | contraception or remain abstinent for 1 month after study drug administration to |
| | prevent the transfer of seminal fluid |
| 10. | Have a stable medical regimen for ≥1 month before randomization (excluded |
| | medications are listed in Exclusion Criteria #9-14) |
| 11. | Able to read and understand study instructions in English, and willing and able to |
| | comply with all study procedures, including returning for follow-up visits, and |
| | participating in standard physical therapy as indicated |

Subjects must NOT have met any of the following exclusion criteria:

| | |
|---|---|
| 1. | Target knee had <100° of flexion, >20° valgus or varus deformity (in the opinion of |
| | the Investigator; if there is any question, an x-ray should be conducted to confirm), |
| | evidence of significant bone loss of ligamentous laxity, or existing hardware that |
| | requires removal during TKA |
| 2. | More than 2 other current focal areas of pain, none greater in intensity than the |
| | target knee and no other active chronic pain conditions that would compromise |
| | operative knee pain evaluation (e.g., CRPS, fibromyalgia) |
| 3. | Inflammatory arthridities (e.g., rheumatoid arthritis, lupus, ankylosing spondylitis, |
| | psoriatic arthritis), with the exception of clinically stable/non-active gout that did not |
| | affect the knee and did not interfere with walking |
| 4. | Undergoing concomitant surgical procedures (in addition to TKA) or non-elective |
| | TKA, or contralateral knee was likely to require TKA within 3 months |
| 5. | Operative arthroscopy in the surgical knee in the last 4 months (except for meniscal |
| | repair which will be no shorter than 6 months before randomization) or in the |
| | contralateral knee in the last 2 months (or 3 months for meniscal repair), or other |
| | prior surgery in either knee in the last 12 months, except for diagnostic arthroscopy |
| 6. | Planned use of any of the following for TKA: general anesthesia using potent |
| | inhalational agents, peripheral nerve block (e.g., femoral nerve block), neuroaxial |
| | (intrathecal or epidural) opioids postoperatively, or knee capsule injections |
| 7. | Known spinal deformities (congenital, degenerative or due to surgery) that would |
| | interfere with standard intrathecal injections at the spinal levels specified in the |
| | protocol (L3/4 or L4/5), or cutaneous infection in the lumbar area that would |
| | preclude intrathecal administration of study drug |
| 8. | Hospitalization or major surgery within 3 months of randomization |
| 9. | Received aspirin or any nonsteroidal anti-inflammatory drug (NSAID) prior to |
| | randomization within the washout period required by the surgical team for surgery |
| | with spinal anesthetic, or planned use of NSAIDS post-operatively through Day 28 |
| 10. | Use of more than 20 mg per day (on average) of oral morphine or its equivalent |
| | within 1 month prior to randomization |
| 11. | Use of adjuvant analgesics for chronic pain control (e.g., gabapentin, pregabalin) |
| | within 2 weeks prior to randomization or planned use post-operatively through Day |
| | 28 |
| 12. | Daily use of benzodiazepines in the 3 months prior to randomization (unless used for |
| | sleep and dosage will be consistent after surgery) |
| 13. | Use of systemic corticosteroids (does not include inhaled steroids) within 3 months |
| | or intra-articular steroid injections within 1 month prior to randomization, or |
| | planned use of either post-operatively through Day 28. |
| 14. | Treatment with immunosuppressives, antipsychotics, anticholinergics, or |
| | anticonvulsants within 1 month of randomization, with the exception of aspirin for |
| | cardiac prophylaxis (as long as discontinued prior to surgery per Exclusion #9) |
| 15. | Hypersensitivity, allergy, or significant reaction to any ingredient of the study drug, |
| | or to anesthetics or analgesics that may be used preoperatively or postoperatively |
| 16. | Current neurologic disorder, which could confound the assessment of pain (e.g., |
| | Parkinson's, Multiple Sclerosis) |
| 17. | Current active depression symptoms |
| 18. | A change in dose or regimen of SSRIs or SNRIs for depression within 1 month of |
| | randomization |
| 19. | Mini Mental Status Exam score <24 at screening |
| 20. | Unstable mental condition which would prevent the patient from understanding the |
| | nature and scope of the study and/or evidence of an uncooperative attitude in the |
| | opinion of the Investigator; subjects diagnosed with schizophrenia, prescribed |
| | antipsychotic medications or MAOIs |
| 21. | Current unstable angina, uncontrolled congestive heart failure, or >1+ pitting edema |
| | of lower extremities or history of acute myocardial infarction, transient ischemic |
| | attack, cerebrovascular accident, pulmonary embolism or seizure within 3 months of |
| | randomization |
| 22. | Severe pulmonary disease; if symptomatic sleep apnea is currently treated with |
| | continuous positive airway pressure (CPAP) or bi-level positive airway pressure |
| | (BIPAP), use must be continued postoperatively at least during the inpatient period. |
| | Patients with sleep apnea associated with a history of postoperative delirium were |
| | excluded. |
| 23. | Current evidence of alcohol abuse or history of alcohol-related complications within |
| | 1 year of randomization including, but not limited to, alcoholic withdrawal seizures, |
| | hallucinations, delirium tremens or detoxification treatment |
| 24. | Known or suspected history of illicit drug abuse within 1 year before randomization, |
| | current or planned use of medical marijuana, or history of opioid dependence within |
| | 2 years before randomization, defined as meeting the DSM-IV TR Criteria for |
| | Substance Dependence |
| 25. | Any malignancy within the past year, with the exception of basal cell carcinoma or |
| | uncomplicated or stable skin cancers documented to not require further or immediate |
| | treatment |
| 26. | Women who are pregnant or nursing |
| 27. | Participation in a clinical trial with the last dose or intervention within 1 month of |
| | randomization |
| 28. | Previous participation in any study involving AYX1 Injection (with exposure to |
| | study drug) |
| 29. | Any condition that, in the opinion of the Investigator, could compromise the safety |
| | of the patient, the patient's ability to comply with study procedures, or the quality of |
| | the data |

Randomization and Masking

Subjects were randomized the day before or the day of surgery. Site management was performed by a clinical research organization (Premier Research International LLC, Philadelphia, Pa., USA), and randomization, data management and analysis were performed centrally (InClin, Inc., San Mateo, Calif., USA). Randomization via electronic data capture system was separated into two stages: a safety stage and the main stage. In the safety stage, the first six subjects were randomized in a 5:1 ratio to the active (AYX1 660 mg in 6 mL) and placebo (6 mL) arms and the second six subjects were randomized in a 5:1 ratio to the active (AYX1 1100 mg in 10 mL) and placebo (10 mL) arms using a centrally generated blocked (block size of 6) randomization sequence. All subjects in the safety stage were injected at the L4/5 level. In the main stage, the remaining 108 subjects were randomized across treatment, dose/volume and injection site. This yielded 10 placebo and 20 AYX1 treated subjects in each of the injection site groups at each dose level, including subjects from the safety stage. A blocked randomization was used with a block size of 12 and randomly ordered ratio of treatment groups within each block.

Study site personnel obtained written consent, enrolled subjects and were also involved in outcome assessments. Anesthesiologists at each hospital received a sterile dosing vial with study drug blinded except for volume. The sponsor, investigator and all study personnel, with the exception of the unblinded pharmacists and the safety monitoring committee, remained blinded to subject treatment assignments until database lock.

Procedures

Study drug at room temperature was administered by lumbar intrathecal injection at either the L3/4 or L4/5 interspace determined by surface landmarks¹⁵ prior to spinal anesthetic (hyperbaric 0.75% bupivacaine, 10-17.5 mg) via the same needle. Subjects were kept seated for approximately two minutes and then placed supine. Intravenous midazolam, propofol and fentanyl were allowed in the preoperative and intraoperative periods. NSAIDs, gabapentinoids, and systemic or injected steroids were prohibited until Day 28. Prophylactic antibiotics, venous thromboembolism prophylaxis, postoperative anti-emesis, knee immobilizers, continuous passive knee motion and physical therapy were allowed as per practitioner standard. Postoperatively subjects were given intravenous opioids to obtain adequate analgesia and then placed on a patient controlled analgesia device until the following morning when a standardized specific range of oral opioid medications was initiated and the amounts recorded. Subjects remained hospitalized for at least 48 hours (to Day 3) after completion of surgery (close of incision); inpatient study assessments were conducted through 72 hours or discharge from the hospital, whichever was earlier.

Perioperative Standard of Care

Surgical Anesthesia/Sedation

Intravenous (IV) midazolam up to 10 mg total was allowed in the preoperative and intraoperative periods. Intraoperative anesthetic consisted of 10-17.5 mg bupivacaine administered in the lumbar intrathecal space following administration of study drug, via the same needle. Intravenous propofol was used for sedation. Intravenous fentanyl dosed in relation to body weight (2-20 μg/kg) was allowed intraoperatively. General anesthesia using potent inhalational agents, femoral nerve blocks, neuroaxial (intrathecal or epidural) opioids, and knee capsule injections were not allowed.

Postoperative Analgesic Options

Postoperative analgesia was 3 5 mg IV morphine followed by 1 2 mg every 5 minutes, titrated to subject requirements. If morphine was not tolerated, alternative analgesia was 0.3 0.5 mg IV hydromorphone (dilaudid) followed by 0.1 mg every 5 minutes. Once pain was controlled, all subjects were started on IV PCA with 2 mg morphine bolus with 10 minute lockout (without basal rate) for a maximum total hourly dose of 12 mg, or 0.2 mg hydromorphone bolus with 10 minute lockout (without basal rate) for a maximum total hourly dose of 1.2 mg. On the morning following surgery, IV PCA was discontinued and a PRN (as needed) oral opioid regimen started consisting of 5 10 mg oxycodone (no more than every 4 hours), which could be increased up to 20 mg every 4 hours temporarily. Alternative opioids were hydromorphone 1 2 mg (no more than every 4 hours), which could be increased up to 4 mg every 4 hours temporarily, or hydrocodone or oxycodone/acetaminophen combinations (up to 10 mg/650 mg) 1-2 doses orally every 4-6 hours (not to exceed 3 g of acetaminophen in 24 hours). Sustained-release opioids (e.g., Oxycontin) were not allowed. The maximum doses of analgesic medications outlined in the protocol were considered a guideline; increased doses of the specified analgesics were allowed if required for clinical care of the subject. Acetaminophen ≤3 g per 24 hours (including acetaminophen in the hydrocodone or oxycodone/acetaminophen combinations, if taken) was permitted for headache or fever post op. Aspirin or other anticoagulant was allowed as per hospital protocol for antiplatelet or anticoagulation; aspirin was also allowed for cardiac prophylaxis (not for analgesia), except prior to randomization within the washout period required before surgery (per Exclusion #9). Other NSAIDs (e.g., Celebrex, Toradol, Motrin) were not allowed prior to randomization within the washout period required before surgery (per Exclusion #9) through Day 28. Other analgesics: Gabapentin (Neurontin) and pregabalin (Lyrica) were not allowed from 2 weeks prior to randomization through Day 28. Use of systemic corticosteroids within 3 months or intra-articular steroid injections within 1 month prior to randomization, or planned use of either post-operatively through Day 28, was not allowed.

Postoperative Care

Use of knee immobilizers, continuous passive motion (CPM), and cooling devices were standardized for study subjects. CPM was used for all study subjects post-operatively through Day 4 (or discharge if earlier). CPM was initiated at 0-60°, with an increase of 10° per day unless the subject was eligible to tolerate more than a 10° increase, or was not able to tolerate a 10° increase. The site's local standard procedures for time to initiate CPM after surgery and daily duration of CPM use were followed, but CPM was discontinued 30 minutes prior to any study efficacy assessments (scheduled NRS pain assessments, ROM assessments, and the walk test). Start and stop times, as well as number of degrees of flexion for CPM were recorded. Ice packs or cooling devices were used if required post-operatively through Day 4 (or discharge if earlier) according to the site's local standard procedures. Use of ice or other cooling devices was discontinued 30 minutes prior to any study efficacy assessments (scheduled NRS pain assessments, ROM assessments, and the walk test). Start and stop times for use of ice packs or other cooling devices during the inpatient stay were recorded (if used). Knee immobilizers were not allowed except during the night during the inpatient stay if required according to the site's local standard procedures. If a knee immobilizer was used it was removed 30 minutes prior to any study efficacy assessments (scheduled NRS pain assessments, ROM assessments, and the walk test). Start and stop times for use of knee immobilizers were recorded (if used).

The study drug (AYX1 or placebo) formulations were isotonic with the cerebrospinal fluid (CSF) at a neutral pH (pH 7-8). The active agent had a concentration of 110 mg/mL AYX1 and an unblinded pharmacist prepared dosing vials within 24 hours of administration.

Trained investigative site personnel performed the outcome tests including pain ratings, knee range of motion (ROM) and walk assessments, during inpatient or clinic visits in a specific sequence.

Pain at rest was rated after at least 30 minutes of rest before performing the walk test. Following the walk test, subjects were asked to rate the average of all pain experienced in the operated knee during the entire walk before knee ROM tests were performed. All pain ratings were assessed by a Likert (0-10) numerical rating scale (NRS). All subjects received training on the efficacy assessments including the NRS pain scale before surgery and were given the opportunity to ask questions. The schedule of assessments through Day 42 is outlined in Table 1.

TABLE 1
Schedule of assessments in the postoperative period

| | | | | | | | | | Follow up Visit | |
|---|---|---|---|---|---|---|---|---|---|---|
| | In Hospital | | | | | | | | Days 7, 14, | |
| | 2 h ± | 4 h ± | 6 h ± | 24 h ± | 28 h ± | 48 h ± | 54 h ± | 72 h (±2 h) | 21, 28 | Day 42 |
| Scheduled Event | 15 m | 15 m | 30 m | 1 h | 1 h | 2 h | 2 h | or Discharge | (±2 days) | (±5 days) |
| Numerical Rating Scale (Likert NRS 0-10) | X | X | X | X | X | X | X | X | X | X |
| Pain Assessment at rest ^{b} | | | | | | | | | | |
| 5 Meter Walk Test with NRS Pain | | | | X | X | X | X | X | | |
| Assessment upon standing for walk and | | | | | | | | | | |
| after walk | | | | | | | | | | |
| 15 Meter Walk Test with NRS Pain | | | | | | | | | X | X |
| Assessment upon standing for walk and | | | | | | | | | | |
| after walk | | | | | | | | | | |
| ROM: Maximum degrees active flexion/ | | X | X | X | X | X | X | X | X | X |
| extension | | | | | | | | | | |
| ROM: Maximum degrees passive flexion/ | | X | X | X | X | X | X | X | X | X |
| extension | | | | | | | | | | |
| ROM: NRS Pain Assessment at 45 degrees | | X | X | X | X | X | X | X | | |
| passive flexion | | | | | | | | | | |
| ROM: NRS Pain Assessment at 90 degrees | | | | X | X | X | X | X | X | X |
| passive flexion | | | | | | | | | | |
| ROM: NRS Pain Assessment at 110 degrees | | | | | | | | | X | X |
| passive flexion | | | | | | | | | (Days 14, 21 | |
| | | | | | | | | | and 28 only) | |

Times indicate the target assessment time +/− allowed time window.

Outcome assessments were analyzed in periods most likely to include all subjects, for inpatient (0-48 hours) and outpatient (7-28 days) periods, as well as a Day 42 follow-up corresponding to the standard postoperative orthopedic visit for TKA. The separation of analysis periods also reflects the expected time to delayed central sensitization and emergence of AYX1 efficacy (>48 hours).¹⁶ The primary efficacy endpoints were mean pain rating with walking during the 5 meter walk tests up to 48 hours and during the 15 meter walk test from 7-28 days. Secondary efficacy endpoints included: mean pain at rest and total use of opioid medications (morphine equivalents) during the inpatient and outpatient periods; degrees of maximum tolerated active knee extension during the inpatient stay; mean pain rating for 45° passive knee flexion during inpatient period; mean pain rating for 90° passive knee flexion during outpatient period; and time to achieve 90° and 110° of active knee flexion.

Safety assessments including physical/neurologic examination (at screening and Day 3), vital signs (screening, Day 1 preoperatively and Day 3), adverse events (AEs) and serious adverse events (SAEs) were collected and recorded by study personnel; SAEs were processed by a central safety-reporting contractor (Premier Research). Blood and urine for clinical laboratory assessments were collected at screening, Day 1 prior to surgery, Day 3 while in the hospital, and at the follow-up visits on Day 7 and Day 28 and processed at a central laboratory (ICON Laboratory Services Inc., Farmingdale, N.Y., USA). Adverse events spontaneously reported by the subject and/or in response to an open non-leading question from study personnel, or revealed by observation at each subject encounter or clinic visit, were recorded along with any concomitant medications or treatments from the time of randomization (AEs) or consent (SAEs) through Day 42. Clinically significant findings from all safety evaluations (physical/neurological examination, vital signs, clinical laboratory assessments, or other assessments done for the clinical care of the subject) were reported as AEs. In the safety stage of the study, safety data for the first two subjects through 48 hours were reviewed by an independent, unblinded safety monitoring committee (SMC) composed of two clinicians not directly involved in the study. If no safety signals were identified by the SMC then the remaining four subjects in the 660 mg cohort were dosed and the data for the entire 660 mg cohort through 48 hours were evaluated by the SMC. This process was repeated for the 1100 mg cohort. The SMC recommendation regarding study continuation and dose escalation was made based on pre-defined safety criteria as outlined in the protocol and the SMC charter. No interim analysis of efficacy was conducted.

Statistical Analysis

The sample size was designed to provide sufficient power within the injection site subgroups as reflected by the L4/5 injection site results from the previous study. Assuming a two point difference in the mean pain rating between treatment groups, a standard deviation of 1·75, 80% power and alpha=0.05, a total of 20 AYX1 and 10 placebo subjects were required. For the analysis across injection site levels, using the same assumptions as for the subgroups, a total of 40 AYX1 subjects and 20 placebo subjects were needed to detect a difference of 1·4 in the mean pain rating at a power of 80%.

All statistical tests were performed at the α=0.05 significance level using two-sided tests, unless otherwise noted. No multiplicity adjustment was applied for the statistical comparisons. Efficacy endpoints were categorized as primary, secondary, and exploratory. No discussion of exploratory endpoints will be presented in this publication. Ad hoc analyses included total use of opioid medications for the follow-up period (28-42 days); a responder analysis, where a responder was defined as a subject who achieved a postoperative pain score of two or lower (corresponding to mild pain) at each assessment time and a determination of the percentage of subjects with pain scores greater than two (moderate or greater pain) at Day 42.

For the primary endpoints, a mixed effects model for repeated measures (MMRM) was applied, with treatment, assessment time point and injection site level as fixed effects, and for the analysis within injection site level with treatment, assessment time point, injection site level and the interaction term of treatment and injection site level as fixed effects. A similar approach was applied for the secondary efficacy endpoints of pain at rest and pain ratings at 45° and 90° passive knee flexion. A compound symmetry covariance structure was used for the model. The least squares means were determined and the treatment differences were tested for statistical significance using the F-test. Additional analyses were completed by injection site level comparing AYX1 to the combined (across injection site level) placebo group. For the mean pain score (NRS) for 45° passive knee flexion 0-48 hours and mean pain score for 90° passive knee flexion 7-28 days analyses utilizing two imputation methodologies for missing data were completed: (1) No imputation only subjects who reached 45° or 90° passive knee flexion (−2°) were included in the analysis or (2) subjects that did not reach 45° or 90° passive knee flexion (−2°) or did not perform the assessment due to pain had the worst possible pain score (10) imputed. For time to achieve 90° and 110° of active knee flexion, subjects not achieving 90° or 110° active knee flexion (−2°) were censored at the date and time of their last ROM test. If the time of the ROM test was missing, the time was imputed as 23:59. Time to 90° and 110° active knee flexion were analyzed using Kaplan-Meier methods. Differences in Kaplan-Meier curves were tested for statistical significance using a stratified log-rank test, stratified for injection site level. Total use of opioid medications was based on conversion of recorded opioid dose to intravenous morphine equivalents and was determined for the hospital stay period 0-48 hours and for the outpatient and follow-up periods 7-28 days and 28-42 days. Differences between treatment groups were determined using an ANOVA with treatment and injection site level as factors. Secondary endpoints were also analyzed within each injection site level. SAS (Version 9.4) was used for all analyses.

The safety population included all randomized subjects who received study drug. The modified intent to treat (mITT) population included all randomized subjects who received the double-blind study drug, underwent TKA, and completed at least one efficacy assessment in the endpoint being analyzed. Summary statistics for vital signs were provided for Day 3 and for the change from baseline to Day 3. Baseline was defined as the last value prior to the study drug administration. Central laboratory data were summarized for each scheduled time point measured and for the change from baseline to each time point. Subject incidences of change in classification with respect to the laboratory normal ranges were summarized in shift tables. Physical and neurological examination findings were summarized by dose and treatment group.

Results (See FIGS. 1-19 for Data Presentation)

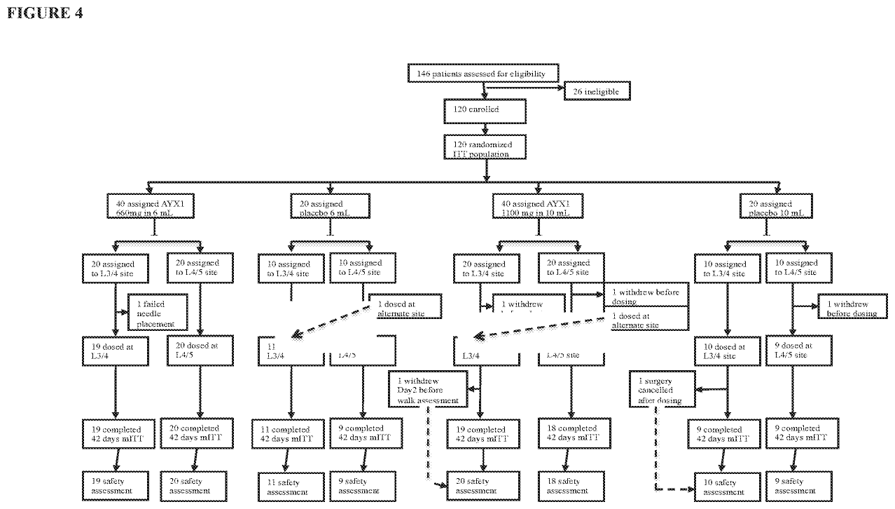

We screened 146 subjects; 26 failed screening assessments and the remaining 120 subjects were randomly allocated to four treatment groups (AYX1 660 mg, AYX1 1100 mg or volume matched placebos) and two injection site level groups (L3/4 or L4/5); 116 were dosed with either AYX1 or placebo and were included in the safety population; one subject withdrew after dosing due to surgeon illness (10 mL placebo) and one withdrew consent before the first walk assessment (AYX1 1100 mg); 114 subjects completed the entire 42-day study (mITT population) (FIG. 4). No safety signals were identified following dosing of the first 12 subjects allowing full enrollment.

The baseline characteristics of the subjects were similar across assigned treatment groups (Table 2). No clinically important differences were noted for duration of surgery or tourniquet time.

TABLE 2
Demographics and intraoperative times

| | AYX1 660 mg | Placebo 6 mL | AYX1 1100 mg | Placebo 10 mL |
|---|---|---|---|---|
| Characteristic | (N = 40) | (N = 20) | (N = 40) | (N = 20) |
| Age - years | 67.5 +/− 7.3 | 65.1 +/− 7.0 | 65.1 +/− 6.1 | 64.7 +/− 6.8 |
| Female sex - N (%) | 21 (52.5%) | 7 (35.0%) | 19 (47.5%) | 8 (40.0%) |
| Race - N (%) | | | | |
| White | 40 (100%) | 19 (95.0%) | 39 (97.5%) | 19 (95.0%) |
| Black | 0 | 0 | 1 (2.5%) | 1 (5.0%) |
| Asian | 0 | 0 | 0 | 0 |
| American Indian or Alaska Native | 0 | 1 (5.0%) | 0 | 0 |
| BMI (kg/m²) | 30.5 (5.0) | 30.8 (3.6) | 31.2 (4.1) | 30.7 (5.0) |
| Postoperative Characteristics | N = 39 | N = 20 | N = 38 | N = 18 |
| Length of surgery (min) | 73.9 (31.5) | 63.9 (23.0) | 69.6 (26.3) | 67.9 (29.1) |
| Length of tourniquet use (min) | 61.6 (20.8) | 55.1 (16.4) | 59.0 (17.4) | 56.7 (20.4) |
| Data are mean (SD) or N (% of total). | | | | |

AYX1 660 mg significantly reduced pain with walking during the outpatient period (LS Mean 2.0 [SEM 0.2] vs. 2.9 [0.3] p=0.026) (FIG. 1) whereas the 1100 mg dose did not statistically separate from placebo (2.4 [0.2] vs. 2.7 [0.4] p=0.423). Neither dose of AYX1 gave significant reduction of pain with walking during the inpatient period [AYX1 660 mg vs. 6 mL placebo (5.1 [0.3] vs. 5.3 [0.4] p=0.646) AYX1 1100 mg vs. 10 mL placebo (4.7 [0.3] vs. 5.6 [0.4] p=0.066)]. When the specific injection site results for AYX1 660 mg were compared to the combined (L3/4 and L4/5) 6 mL placebo, there were no clinically important differences in efficacy between the AYX1 660 mg injection site groups for pain reduction (Table 3).

TABLE 3
Primary and secondary endpoint efficacy endpoints

| | | | | | | |
|---|---|---|---|---|---|---|
| | | | | Diff of | | % |
| | | | | means | | Reduction |
| Group | Time | AYX1 | Placebo | (95% CI) | p | of pain |

Primary Endpoint
Pain with Walking LS Mean (SEM)

| N | | | 39 | | 20 | | | | | |
|---|---|---|---|---|---|---|---|---|---|---|
| 660 mg | 0-48 | hours | 5.1 | (0.3) | 5.3 | (0.4) | −0.2 | (−1.11, 0.69) | 0.646 | 4% |
| 660 mg | 7-28 | days | 2.0 | (0.2) | 2.9 | (0.3) | −0.9 | (−1.76, −0.12) | 0.026 | 31% |
| N | | | 37 | | 18 | | | | | |
| 1100 mg | 0-48 | hours | 4.7 | (0.3) | 5.6 | (0.4) | −0.9 | (−1.82, 0.06) | 0.066 | 16% |
| 1100 mg | 7-28 | days | 2.4 | (0.2) | 2.7 | (0.4) | −0.3 | (−1.21, 0.51) | 0.423 | 11% |
| N | | | 19 | | 20 | | | | | |
| 660 mg L3/4 | 7-28 | days | 2.1 | (0.4) | 3.0 | (0.4) | −0.9 | (−1.90, 0.19) | 0.099 | 30% |
| combined placebo | | | | | | | | | | |
| N | | | 20 | | 20 | | | | | |
| 660 mg L4/5 | 7-28 | days | 1.9 | (0.3) | 3.0 | (0.3) | −1.0 | (−1.97, 0.12) | 0.037 | 33% |
| combined placebo | | | | | | | | | | |

Secondary Endpoints
Pain at Rest LS Mean (SEM)

| N | | | 39 | | 20 | | | | | |
|---|---|---|---|---|---|---|---|---|---|---|
| Across injection | 7-28 | days | 1.5 | (0.2) | 2.4 | (0.3) | −0.9 | (−1.68, 0.07) | 0.033 | 38% |
| sites | | | | | | | | | | |
| N | | | 19 | | 20 | | | | | |
| 660 mg L3/4 | 7-28 | days | 1.4 | (0.3) | 2.4 | (0.3) | −1.0 | (−1.95, 1.04) | 0.044 | 42% |
| combined placebo | | | | | | | | | | |
| N | | | 20 | | 20 | | | | | |
| 660 mg L4/5 | 7-28 | days | 1.6 | (0.4) | 2.4 | (0.4) | −0.8 | (−1.75, 0.19) | 0.126 | 33% |
| combined placebo | | | | | | | | | | |

Opioid Utilization mg IV morphine equivalents LS mean (SEM)

| N | | | 39 | | 20 | | | | | |
|---|---|---|---|---|---|---|---|---|---|---|
| | 0-48 | hours | 85.4 | (6.2) | 77.0 | (8.7) | 8.4 | (−12.76, 29.55) | 0.433 | NA |
| N | | | 39 | | 20 | | | | | |
| | 7-28 | days | 195.0 | (25.3) | 207.9 | (35.3) | −12.9 | (98.99, 73.13) | 0.766 | NA |
| N | | | 39 | | 20 | | | | | |
| | 28-42 | days | 50.7 | (10.6) | 51.6 | (14.7) | −0.9 | (36.83, 35.08) | 0.962 | NA |

NRS score passive knee flexion (Imputation 1) LS Mean (SEM)

| N | | | 39 | | 20 | | | | | |
|---|---|---|---|---|---|---|---|---|---|---|
| 45° | 0-48 | hours | 5.7 | (0.3) | 5.5 | (0.4) | 0.21 | (−0.78, 1.21) | 0.670 | −4% |
| N | | | 39 | | 20 | | | | | |
| 90° | 7-28 | days | 4.4 | (0.4) | 5.0 | (0.6) | −0.67 | (−2.06, 0.71) | 0.338 | 13% |

Maximum degrees of active extension (SD)

| N | | | 38 | | 20 | | | | |
|---|---|---|---|---|---|---|---|---|---|
| | 0-48 | hours | −10.3 | (5.6) | −9.6 | (6.5) | −0.74 | (−4.24, 2.76) | NA |

Time (days) to achieve active knee flexion median (25-75^{th} percentile)

| N/censored | 39/0 | | 20/2 | (10%) | | | |
|---|---|---|---|---|---|---|---|
| 90° | 15.0 | (8.0-16.0) | 8-0 | (2.0-25.5) | NA | 0.854 | NA |
| N | 39/12 | (30.8%) | 20/7 | (35%) | | | |
| 110° | 42.0 | (29.0-45.0) | 43.0 | (23.0-44.0) | NA | 0.852 | NA |

Table 3: Summary data for primary endpoint pain with walking and secondary endpoints of pain at rest, opioid utilization, maximal degrees of active extension, mean NRS score for passive knee flexion at 45° or 90° and time to achieve 90° and 110° active knee flexion. Data for pain with walking and pain at rest are LS Mean (SEM) calculated for the time intervals indicated using a mixed effects model for repeated measurements (MMRM) with treatment assessment time and injection site as covariates and the interaction term of treatment and injection site as fixed effects. For pain with walking, analyses with Imputation 1 are shown—if the subject did not do the walk test due to pain or did not complete the entire walk distance due to pain, the data were imputed as the worst possible pain score of 10. For mean pain at rest (NRS) during hospital stay 0-48 hours and after hospital discharge (7-28 days), if the subject did not complete the NRS at rest at a specific time point the missing data were not imputed. For opioid utilization during the hospital stay, site staff recorded each dose of analgesic medication, after discharge through Day 42, the total daily dose of analgesic medication was recorded by the subject using a paper diary. NA Not applicable.

Having established the effective dose of 660 mg AYX1 for the primary endpoint, all results in this report (except for safety data) will concern the AYX1 660 mg group.

AYX1 660 mg reduced pain scores at rest during the outpatient period when compared to placebo 6 mL across injection groups (LS Mean 1·5 [SEM 0·2] vs. 2·4 [0·3] p=0·033 and difference −0·9 [95% CI−1·68, 0·07] Table 3). The reduction in pain compared to placebo was 38% on top of standard of care. Opioid utilization did not differ between the AYX1 660 mg and placebo groups either at the 0-48 hour or 7-28 day periods (Table 3). AYX1 660 mg, when compared with placebo 6 mL, did not significantly reduce pain with knee flexion (45°, 90° or 110°) nor reduce time to achieve ROM endpoints (Table 3).

Table 4 below illustrates primary endpoint and pain at rest secondary endpoint displayed with only specific injection level placebo.

TABLE 4

| | | | | | | |
|---|---|---|---|---|---|---|
| | | | | | | % |
| | | | | Diff of means | | Reduction |
| Group | Time | AYX1 | Placebo | (95% CI) | p | of pain |

Primary Endpoint Pain with Walking

| N | | 19 | | 11 | | | | | |
|---|---|---|---|---|---|---|---|---|---|
| 660 mg L3/4 | 7-28 days | 2.1 | (0.3) | 3.5 | (0.5) | −1.4 | (−2.51, 0.24) | 0.018 | 40% |
| N | | 20 | | 9 | | | | | |
| 660 mg L4/5 | 7-28 days | 1.9 | (0.3) | 2.3 | (0.5) | −0.4 | (−1.61, 0.79) | 0.499 | 17% |

Secondary Endpoint Pain at Rest

| N | | 19 | | 11 | | | | | |
|---|---|---|---|---|---|---|---|---|---|
| 660 mg L3/4 | 7-28 days | 1.4 | (0.3) | 2.9 | (0.4) | −1.5 | (−2.61, 0.41) | 0.008 | 52% |
| N | | 20 | | 9 | | | | | |
| 660 mg L4/5 | 7-28 days | 1.6 | (0.3) | 1.8 | (0.5) | −0.1 | (−1.295, 1.036) | 0.827 | 11% |

Data are Least Square Means (SEM), difference of means for AYX1 versus placebo (95% CI) or percent reduction of pain compared to placebo.

The AEs experienced by subjects in the safety population over the 42-day study period are displayed in Table 5.

TABLE 5

| | AYX1 660 mg | Placebo 6 mL | AYX1 1100 mg | Placebo 10 mL |
|---|---|---|---|---|
| Adverse Event (AE) | (N = 39) | (N = 20) | (N = 38) | (N = 19) |
| Subjects experiencing any AE | 39 (100) | 19 (95.0) | 38 (100) | 18 (94.7) |
| AEs leading to discontinuation | 0 | 0 | 0 | 0 |
| Subjects experiencing Serious | 3 (7.7) | 2 (10.0) | 6 (15.8) | 0 |
| AEs | | | | |
| Number of SAEs | 3 | 3 | 7 | 0 |
| Deaths | 0 | 0 | 0 | 0 |
| AEs reported in ≥ 10% | | | | |
| subjects in any group | | | | |
| Nausea | 30 (76.9) | 13 (65.0) | 30 (78.9) | 14 (73.7) |
| Constipation | 18 (46.2) | 9 (45.0) | 16 (42.1) | 8 (42.1) |
| Vomiting | 17 (43.6) | 7 (35.0) | 12 (31.6) | 11 (57.9) |
| Procedural hypotension | 12 (30.8) | 1 (5.0) | 14 (36.8) | 3 (15.8) |
| Decreased appetite | 10 (25.6) | 3 (15.0) | 7 (18.4) | 5 (26.3) |
| Dizziness | 9 (23.1) | 4 (20.0) | 6 (15.8) | 3 (15.8) |
| Insomnia | 8 (20.5) | 4 (20.0) | 7 (18.4) | 2 (10.5) |
| Crystal urine present | 6 (15.4) | 2 (10.0) | 8 (21.1) | 3 (15.8) |
| Pruritus | 7 (17.9) | 3 (15.0) | 5 (13.2) | 6 (31.6) |
| Pyrexia | 6 (15.4) | 2 (10.0) | 6 (15.8) | 2 (10.5) |
| Anxiety | 3 (7.7) | 2 (10.0) | 7 (18.4) | 1 (5.3) |
| Blood pressure increased | 4 (10.3) | 1 (5.0) | 5 (13.2) | 1 (5.3) |
| Headache | 8 (20.5) | 4 (20.0) | 1 (2.6) | 3 (15.8) |
| Hypertension | 1 (2.6) | 0 | 8 (21.1) | 2 (10.5) |
| Post procedural shivering | 5 (12.8) | 1 (5.0) | 4 (10.5) | 0 |
| Anemia | 4 (10.3) | 1 (5.0) | 4 (10.5) | 3 (15.8) |
| Fatigue | 4 (10.3) | 1 (5.0) | 3 (7.9) | 1 (5.3) |
| Hypotension | 3 (7.7) | 1 (5.0) | 3 (7.9) | 3 (15.8) |
| Hyperhidrosis | 2 (5.1) | 0 | 4 (10.5) | 3 (15.8) |
| Hyponatremia | 5 (12.8) | 0 | 1 (2.6) | 1 (5.3) |
| Muscle spasms | 3 (7.7) | 2 (10.0) | 3 (7.9) | 0 |
| Anxiety postoperative | 1 (2.6) | 2 (10.0) | 4 (10.5) | 0 |
| Depression | 1 (2.6) | 0 | 4 (10.5) | 1 (5.3) |
| Anemia postoperative | 3 (7.7) | 2 (10.0) | 1 (2.6) | 0 |
| Gastritis | 2 (5.1) | 2 (10.0) | 2 (5.3) | 1 (5.3) |
| Hypokalemia | 2 (5.1) | 2 (10.0) | 2 (5.3) | 1 (5.3) |
| Back pain | 1 (2.6) | 1 (5.0) | 2 (5.3) | 4 (21.1) |
| Pain in extremity | 2 (5.1) | 2 (10.0) | 1 (2.6) | 1 (5.3) |
| Diarrhea | 2 (5.1) | 3 (15.0) | 0 | 1 (5.3) |
| Incision site cellulitis | 0 | 2 (10.0) | 2 (5.3) | 0 |
| Rash | 0 | 2 (10.0) | 2 (5.3) | 0 |
| Arthralgia | 0 | 3 (15.0) | 1 (2.6) | 2 (10.5) |
| Somnolence | 0 | 2 (10.0) | 0 | 0 |

Table 4: The number and percentage of treatment-emergent adverse events experienced by at least 10% of subjects in any group up to Day 42. Data are presented as N (%) for each treatment group by appropriate MedDRA term. Subjects reporting the same adverse event (MedDRA preferred term) more than once were counted only once. A diagnosis of the adverse event based on the presenting signs, symptoms, and/or other clinical information, but not the individual signs/symptoms/clinical information was documented as the AE. For each AE, the investigator reported the onset and resolution date and time (or date and time of stabilization if not resolved), maximum intensity, causality, action taken, outcome, seriousness, and whether or not the event caused the subject to discontinue the study. Adverse events with an onset date/time prior to randomization and SAEs with an onset date/time prior to the time of consent were recorded as medical history.

The subset of AEs experienced in the 24 hours following study drug dosing is presented in Table 6.

TABLE 6
AEs in First 24 Hours

| | AYX1 660 mg | Placebo 6 mL | AYX1 1100 mg | Placebo 10 mL |
|---|---|---|---|---|
| Adverse Event (AE) | (N = 39) | (N = 20) | (N = 38) | (N = 19) |
| Subjects experiencing any AE in | 36 (92.3) | 16 (80.0) | 36 (94.7) | 17 (89.5) |
| first 24 hours | | | | |
| AEs reported in ≥ 10% subjects | | | | |
| in any group | | | | |
| Nausea | 27 (69.2) | 11 (55.0) | 28 (73.7) | 13 (68.4) |
| Vomiting | 12 (30.8) | 6 (30.0) | 8 (21.1) | 8 (42.1) |
| Procedural hypotension | 12 (30.8) | 1 (5.0) | 14 (36.8) | 3 (15.8) |
| Decreased appetite | 7 (17.9) | 2 (10.0) | 6 (15.8) | 4 (21.1) |
| Hypertension | 1 (2.6) | 0 | 7 (18.4) | 2 (10.5) |
| Constipation | 6 (15.4) | 2 (10.0) | 4 (10.5) | 3 (15.8) |
| Post procedural shivering | 5 (12.8) | 1 (5.0) | 4 (10.5) | 0 |
| Insomnia | 2 (5.1) | 2 (10.0) | 4 (10.5) | 0 |
| Pruritus | 3 (7.7) | 1 (5.0) | 3 (7.9) | 2 (10.5) |
| Anxiety | 1 (2.6) | 1 (5.0) | 4 (10.5) | 0 |
| Hypotension | 1 (2.6) | 0 | 1 (2.6) | 2 (10.5) |
| Dizziness | 2 (5.1) | 2 (10.0) | 2 (5.3) | 1 (5.3) |
| Headache | 1 (2.6) | 2 (10.0) | 0 | 0 |
| Anxiety postoperative | 1 (2.6) | 2 (10.0) | 3 (7.9) | 0 |

Table 6: The number and percentage of adverse events experienced by at least 10% of subjects in the first 24 hours following study drug dosing; data are presented as N (%) for each treatment group by appropriate MedDRA term. Subjects reporting the same adverse event (MedDRA preferred term) more than once were counted only once.

Procedural hypotension and postoperative shivering were reported more often in both the AYX1 groups compared to placebo but were transient and managed with vasopressor medications. Hypertension, anxiety and postoperative anxiety were reported more often in the AYX1 1100 mg group compared to placebo 10 mL. The overall AE profile was 90% mild to moderate with no AE clearly associated with AYX1. A total of 11 subjects experienced 13 serious adverse events (Table 7).

TABLE 7
Serious treatment emergent adverse events

| Treatment | | | |
|---|---|---|---|
| Group | Preferred Term | Intensity | Onset day |
| Placebo 6 mL | Pulmonary edema | Severe | 40 |
| | Respiratory failure | Severe | 41 |
| Placebo 6 mL | Aspiration | Moderate | 3 |
| | pneumonia | | |
| AYX1 660 mg | Deep vein | Mild | 3 |
| | thrombosis | | |
| AYX1 660 mg | Deep vein | Moderate | 23 |
| | thrombosis | | |
| AYX1 660 mg | Dysphagia | Severe | 21 |
| AYX1 1100 mg | Atrial fibrillation | Moderate | 3 |
| AYX1 1100 mg | Postoperative ileus | Moderate | 3 |
| AYX1 1100 mg | Hypoxemia | Moderate | 1 |
| AYX1 1100 mg | Abdominal pain | Mild | 4 |
| | upper | Severe | 17 |
| | Constipation | | |
| AYX1 1100 mg | Dyspnea | Mild | 26 |
| AYX1 1100 mg | Pyrexia | Moderate | 1 |

All SAEs were considered unrelated to study drug and all resolved. All SAEs were rated mild or moderate except for two severe SAEs in a single placebo 6 mL treated subject (pulmonary edema and respiratory failure starting 40 days after dosing), one in an AYX1 1100 mg treated subject (constipation starting 17 days after dosing) and one in an AYX1 660 mg treated subject (dysphagia starting 21 days after dosing).

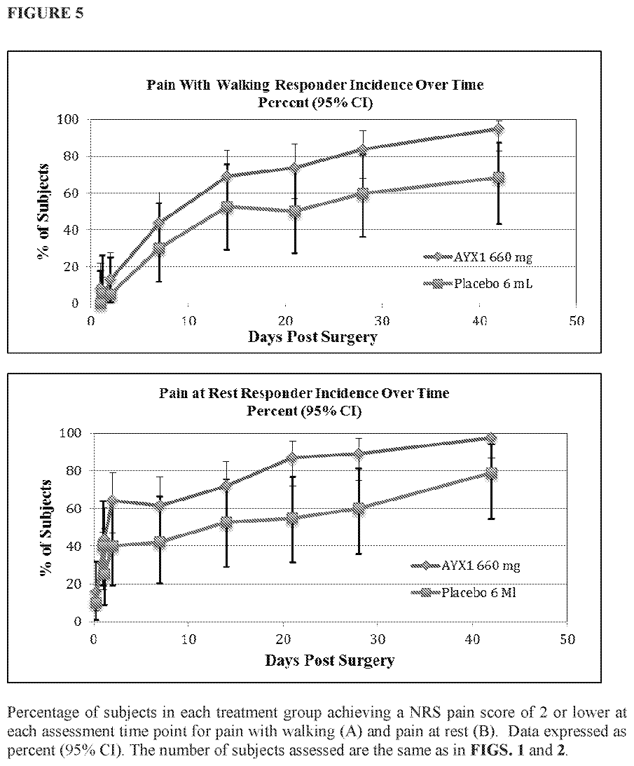

A post hoc responder analysis of the individual time point data for pain with walking and pain at rest was performed. A responder was defined as a subject who achieved a postoperative pain score of two or lower out of 10 (mild pain) as displayed in FIG. 5. AYX1 produced a higher percentage of responders over placebo at every time point after 48 hours. A similar result was observed with pain at rest with AYX1 giving more than 60% response on average by 48 hours and continuing to exceed the rate of placebo for the entire 42-day period. To assess the potential of AYX1 for preventing chronic pain using an intermediate time surrogate at Day 42, the percentage of subjects with pain scores greater than two out of 10 (moderate or greater pain) at Day 42 are displayed in FIG. 3. For both pain with walking and pain at rest the incidence of pain scores greater than two at Day 42 was greater in the placebo group compared to AYX1 660 mg. Relative risk compared to placebo was 0.16 for walking and 0.12 for pain at rest.

Discussion

A single intrathecal injection of AYX1 660 mg prior to TKA surgery reduced postoperative pain with walking for at least 42 days following TKA compared to placebo when added to standard of care. AYX1 reduction of postoperative movement-evoked pain was fully developed after 48 hours. Subjects treated with AYX1 reported a mean pain rating of 0·9 NRS units or 31% lower than placebo for the period 7-28 days following surgery. Additionally AYX1 reduced outpatient pain at rest compared to placebo and markedly reduced the percentage of subjects experiencing moderate or greater pain at Day 42, a surrogate for chronic pain seen at three months or longer.^{17,18} No significant difference was noted for opioid consumption or ROM assessments in either time period. The fully developed separation of AYX1 660 mg from placebo 6 mL after 48 hours is directly correlated with its mechanism of action as EGR1 controls the delayed post-traumatic (surgery) forms of neuronal sensitization.^{9,16,19} This study achieved its three objectives of determining safety in the TKA population, selecting an optimal dose (660 mg) and determining that intrathecal injection level was not critical to efficacy of AYX1 at dose/volumes higher than 330 mg in 3 mL.

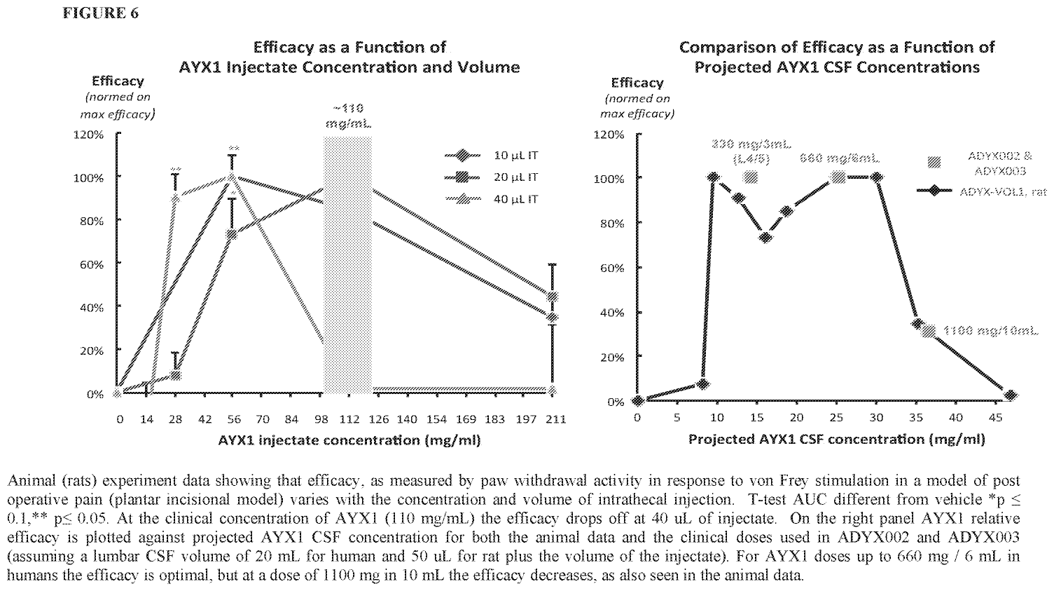

The lack of significant pain reduction observed at the 1100 mg dose is consistent with preclinical pharmacological studies where AYX1 efficacy was lower at high projected CSF concentrations (FIG. 6). In preclinical pharmacokinetic studies, the AYX1 CSF half-life appeared shorter and nuclease-based degradation was faster at high AYX1 CSF concentrations. These combined data suggest a suboptimal EGR1 inhibition and a reduced efficacy above a certain initial AYX1 concentration.

The safety profile of AYX1 in this study was consistent with TKA surgery performed under spinal anesthesia with standard of care perioperative pain management and is also consistent with previous clinical and animal toxicology studies.¹¹⁻¹³ Procedural hypotension was mild to moderate and managed by vasopressors. The rate of procedural hypotension (˜30%) is consistent with the spinal anesthesia literature.²² No reports of hypotension occurred in the phase 1 study in the absence of intrathecal local anesthetic. Post-procedural shivering was likewise mild to moderate, transient and managed by intravenous meperidine. Given the short half-life of AYX1 we also focused on the AEs experienced in the first 24 hours following study drug dosing. Aside from procedural hypotension and post-procedural shivering discussed above, hypertension, anxiety and postoperative anxiety were experienced more often in the AYX1 1100 mg group compared to placebo 10 mL group. Of the seven subjects experiencing mild or moderate hypertension in the first 24 hours, three received perioperative vasopressors and an additional three decreased or stopped antihypertensive medications prior to surgery. Of the seven subjects experiencing mild or moderate anxiety or postoperative anxiety three were given metoclopramide or promethazine (associated with anxiety AEs), two were given perioperative adrenergic agonists and two had a prior history of anxiety or mood disorder. These concomitant medications and history suggest multifactorial contributions to the AEs reported. The safety profile will be further assessed in subsequent phase 3 studies.

Clinically relevant reduction in pain has been defined as a 30 to 50% reduction from baseline.²³ This reduction from baseline by necessity includes the placebo response and the difference between active and placebo can be much less than 30%. The preventive nature and design of this study required that the data be analyzed and expressed as a difference between treated groups without reference to a pre-dose baseline. In that context, the >30% reduction in pain observed in this study relative to placebo when added to standard of care demonstrates a robust clinical relevance and impact for patients.

Further, pain with movement (walking) is an unaddressed clinical problem²⁴ and is more resistant to opioid analgesia than is pain at rest.²⁵ AYX1 is therefore providing a benefit not obtainable with opioid therapy. Opioid analgesic use was collected to evaluate possible confounds to pain assessments from potential differential opioid use and not to assess opioid sparing; surgeons were allowed to prescribe scheduled opioid intake per standard of care. AYX1 clinical relevance is further supported by the higher percentage of subjects compared to placebo achieving a pain score of two or less (mild pain) both with walking and at rest at any recorded time point during the outpatient period.²⁶

The marked difference between AYX1 and placebo treated subjects in the incidence of moderate or greater pain for both pain with walking and pain at rest would be expected to continue out to 12 weeks. Pain at 30-42 days is considered predictive of chronic pain at six months.^{17,18} These six-week data are thus considered relevant and efficient surrogates for the data that would be achieved in longer studies. The long follow up period after study drug administration corresponds to many half-lives of AYX1 elimination and fits the definition of preventive therapy.²⁸

This study has several strengths to support these conclusions: we employed a limited number of sites to reduce variability and the efficacy assessments were carried out under the supervision and participation of trained site personnel; the duration and frequency of postoperative assessment was longer at 42 days than most published studies of postoperative pain; and the assessments were carried out at times far removed from the administration of study drug therefore separating treatment and assessment. In a study enrolling a similar population of patients undergoing TKA, approximately 68% reported moderate to severe pain when walking at 30 days post surgery,²⁷ higher than the placebo rate in the current study.

The limitations of the trial were predominantly due to pragmatic trial design decisions such as the sample size and the use of point estimate for efficacy assessments to ensure consistent measures but was offset by the use of a repeat measures analysis to employ all data within the time period of concern. The postoperative treatment was consistent with standard of care for TKA except that we prohibited major nerve block or local anesthetic infiltration of the wound to minimize the influence of variable technique. Even with these considerations the placebo-treated subjects in this study had pain at rest scores at 7 and 30 days equivalent to published reports of similarly treated patients while the pain with walking in the placebo groups was similar at 7 days but lower at 30 days²⁷ The estimation of chronic pain prevention was suggestive at 6 weeks but would require a follow-up period of at least 12 weeks. The design of this study has addressed several sources of bias including: selection (random sequence generation and allocation concealment within a volume of injection), performance (blinding of participants and personnel), detection (blinding of outcome assessment in particular the lack of association between administration and assessment of outcome), attrition (98% of dosed subjects contributed data to the primary endpoint), and reporting (outcomes are clinically meaningful and validated). A potential remaining source of bias is due to the sample size.

In summary, a single preoperative intrathecal treatment with the EGR1 transcription factor decoy AYX1 at 660 mg was well tolerated and significantly reduced movement-evoked pain from Day 7 through 28, and the pain relief persisted through the entire 42-day follow up period. The pain relief observed when added to standard of care. This study established 660 mg as the optimal AYX1 dose and demonstrated that the effect was not dependent upon lumbar interspace injection site. Additionally, pain at rest was significantly reduced after 48 hours with AYX1 compared to placebo and the effect was maintained for the entire 42-day assessment period. Analysis of the proportion of subjects with moderate or greater pain at Day 42 provides strong support for AYX1 prevention of chronic pain. The pain modulating effects of AYX1 on delayed sensitization are specific as demonstrated by the marked effect beyond 48 hours and sustained well beyond the CSF half-life of AYX1. The unique nature of AYX1-induced inhibition of central sensitization and the duration of its effects places AYX1 in a distinct class of pain response modifiers.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as, an acknowledgment or any form of suggestion that they constitute valid prior art or form part of the common general knowledge in any country in the world.

### REFERENCES

1 Andersen L O, Gaarn-Larsen L, Kristensen B B, Husted H, Otte K S, Kehlet H. Subacute pain and function after fast-track hip and knee arthroplasty. Anaesthesia 2009 May; 64:508-513.
2 Capdevila X, Barthelet Y, Biboulet P, Ryckwaert Y, Rubenovitch J, d'Athis F. Effects of perioperative analgesic technique on the surgical outcome and duration of rehabilitation after major knee surgery. Anesthesiology 1999; 91:8-15.
3 Kehlet H, Jensen T S, Woolf C J. Persistent postsurgical pain: risk factors and prevention. Lancet 2006; 367:1618-1625.
4 Parvizi J, Miller A G, Gandhi K. Multimodal pain management after total joint arthroplasty. J Bone Joint Surg Am 2011; 93:1075-1084.
5 Beckmann A M and Wilce P A. Review: Egr transcription factors in the nervous system. Neurochem Int 1997; 31:477-510.
6 Herdegen T, Fiallos-Estrada C E, Schmid W, Bravo R, Zimmermann M. The transcription factors c-JUN, JUN D and CREB, but not FOS and KROX-24, are differentially regulated in axotomized neurons following transection of rat sciatic nerve. Brain Res Mol Brain Res 1992; 14:155-165.
7 Liang F and Jones E G. Developmental and stimulus-specific expression of the immediate-early gene zif268 in rat spinal cord. Brain Res 1996; 729:246-252.
8 Rahman O I, Terayama R, Ikeda T, Koganemaru M, Nakamura T, Shiba R, Nishimori T. Differential effects of NMDA and AMPA/KA receptor antagonists on c-Fos or Zif/268 expression in the rat spinal dorsal horn induced by noxious thermal or mechanical stimulation, or formalin injection. Neurosci Res 2002; 43:389-399.
9 Rygh L J, Suzuki R, Rahman W, Wong Y, Vonsy J L, Sandhu H, Webber M, Hunt S, Dickenson A H. Local and descending circuits regulate long-term potentiation and Zif268 expression in spinal neurons. Eur J Neurosci 2006; 24:761-772.
10 Ko S W, Vadakkan K I, Ao H, Gallitano-Mendel A, Wei F, Milbrandt J, Zhuo M. Selective contribution of Egr1 (Zif/268) to persistent inflammatory pain. J Pain 2005; 6:12-20.
11 Mamet J, Klukinov M, Yaksh T L, Malkmus S A, Williams S, Harris S, Manning D C, Taylor B K, Donahue R R, Porreca F, Xie J Y, Oyarzo J, Brennan T J, Subieta A, Schmidt W K, Yeomans D C. Single intrathecal administration of the transcription factor AYX1 prevents acute and chronic pain after incisional, inflammatory, or neuropathic injury. Pain 2014; 155:322-333.
12 Manning D, Hebert K, Harris S, Schmidt W K, Mamet J. ADYX001: a phase 1 safety study of AYX1, an intrathecally administered transcription factor decoy against EGR1 intended to reduce acute and prevent persistent postoperative pain. Anesthesiology ASA abstract # A₃₁₅₇.
   http://www.asaabstracts.com/strands/asaabstracts/abstract.htm;jsessionid=ECB2814A₉₃B09F 1EF485AC63DF949818?year=2013&index=17&absnum=3120
13 Manning D, Hebert K, Harris S, et. al. ADYX002: a phase 2a study to evaluate safety and efficacy of a preoperative dose of transcription factor decoy AYX1 to prevent pain after total knee arthroplasty. Abstract presented at the American Society of Regional Anesthesia and Pain Management 13th Annual Pain Medicine Meeting, San Francisco 2014 unpublished.
14 Lunn T H, Kristensen B B, Andersen A O, Husted H, Otte K S, Gaarn-Larsen L. Effect of high-dose preoperative methylprednisolone on pain and recovery after total knee arthroplasty: a randomized, placebo-controlled trial. British J Anaesthesia 2011; 106: 230-8.
15 Chakraverty R, Pynsent P, Isaacs K. Which spinal levels are identified by palpation of the iliac crests and the posterior superior iliac spines? J Anat 2007; 210: 232-236.
16 Ferarri L F, Bogen O, Reichling D B, Levine J D. Accounting for the delay in the transition from acute to chronic pain: axonal and nuclear mechanisms. J Neurosci 2015; 35:495-507.
17 Page M G, Katz J, Escobar EMR, Lutzky-Cohen N, Curtis K, Fuss S, Clarke H A. Distinguishing problematic from nonproblematic postsurgical pain: a pain trajectory analysis after total knee arthroplasty. Pain 2015; 156: 460-468.
18 Aasvang E K, Gmaehle E, Hansen J B, Gmaehle B, Forman J L, Schwarz J, Bittner R, Kehlet H. Predictive risk factors for persistent postherniotomy pain. Anesthesiology 2010; 112: 957-969.
19 Jones M W, Errington M L, French P J, Fine A, Bliss T V, Garel S, Charnay P, Bozon B, Laroche S, Davis S. A requirement for the immediate early gene Zif268 in the expression of late LTP and long-term memories. Nat Neurosci 2001; 4:289-296.
20 Artru A A. Spinal cerebrospinal fluid chemistry and physiology. In: Yaksh T L, ed. Spinal Drug Delivery. Amsterdam: Elsevier Science B.V.; 1999. pp. 177-237.
21 Edsbagge M, Starck G, Zetterberg H, Ziegelitz D, Wikkelson C. Spinal cerebrospinal fluid volume in healthy elderly individuals. Clin Anat 2011; 24:733-740.
22 Carpenter R L, Caplan R A, Brown D L, Stephenson C, Wu R. Incidence and risk factors for side effects of spinal anesthesia. Anesthesiology 1992; 76: 906-916.
23 Farrar J T, Berlin J A, Strom B L. Clinically important changes in acute pain outcome measures: a validation study. J Pain Symptom Manage 2003; 25: 406-411.
24 Srikandarajah S, Gilron I. Systematic review of movement-evoked pain versus pain at rest in postsurgical clinical trials and meta-analyses: a fundamental distinction requiring standardized measurement. Pain 2011; 152:1734-1739.
25 Tverskoy M, Oren M, Dashkovsky I, Kissin I. Alfentanil dose-response relationships for relief of postoperative pain. Anesth Analg 1996; 83:387-93.
26 Ruyssen-Witrand A, Tubach F, Ravaud. Systematic review reveals heterogeneity in definition of a clinically relevant difference in pain. J Clin Epidemiol 2011; 64: 463-470.
27 Anderson L O, Gaarn-Larsen L, Kristensen B B, Husted H, Otte K S, Kehlet H. Subacute pain and function after fast-track hip and knee arthroplasty. Anaesthesia 2009; 64: 508-513.
28 Katz J, McCartney C J L. Current status of pre-emptive analgesia. Current Opinion in Anesthesiology 2002; 15: 435-441.
29 Andreae M H, Andreae D A. Regional anaesthesia to prevent chronic pain after surgery: a Cochrane systematic review and meta-analysis. Br J Anaesth 2013; 111: 711-720.
30 Chaparro L E, Smith S A, Moore R A, Wiffen P J, Gilron I. Pharmacotherapy for the prevention of chronic pain after surgery in adults. Cochrane Database of Systematic Reviews 2013; 7: CD008307.

1. A method for reducing acute pain, and/or preventing chronic pain, in a patient undergoing surgery, comprising:
a. administering a single perioperative intrathecal injection of an effective amount of an oligonucleotide decoy, comprising one or more binding sites for EGR1, to a patient in need thereof, wherein the effective amount of the oligonucleotide decoy is from about 330 mg to about 660 mg.

2. The method according to claim 1, wherein the patient is undergoing a lower extremity surgery.

3. The method according to claim 1, wherein the patient is undergoing an upper body surgery.

4. The method according to claim 1, wherein the patient is undergoing a mid-body or abdominal surgery.

5. The method according to claim 1, wherein the patient is undergoing a knee surgery.

6. The method according to claim 1, wherein the patient is undergoing a total knee arthroplasty.

7-13. (canceled)

14. The method according to claim 1, wherein the oligonucleotide decoy comprises SEQ ID NO. 42.

15-18. (canceled)

19. The method according to claim 1, wherein the effective amount of the oligonucleotide decoy is about 330 mg/3 mL.

20. (canceled)

21. The method according to claim 1, wherein the effective amount of the oligonucleotide decoy is about 660 mg/6 mL.

22. The method according to claim 1, wherein the patient experiences a statistically significant or clinically effective reduction in pain through at least day 28 post-surgery.

23-24. (canceled)

25. The method according to claim 1, wherein the patient experiences a statistically significant or clinically effective reduction in movement-evoked pain from about day 7 post-surgery through at least day 28 post-surgery.

26. The method according to claim 1, wherein the patient experiences a statistically significant or clinically effective reduction in pain at rest from about day 7 post-surgery through at least day 28 post-surgery.

27-30. (canceled)

31. The method according to claim 1, wherein the patient experiences a statistically significant reduction in movement-evoked pain from about day 7 post-surgery through at least day 28 post-surgery, wherein said reduction in pain is at least an additional 30% reduction in pain experienced by said patient as compared to a patient not administered the oligonucleotide decoy.

32. The method according to claim 1, wherein the patient experiences a statistically significant reduction in pain at rest from about day 7 post-surgery through at least day 28 post-surgery, wherein said reduction in pain is at least an additional 30% reduction in pain experienced by said patient as compared to a patient not administered the oligonucleotide decoy.

33-36. (canceled)

37. The method according to claim 1, wherein the patient experiences a reduction in movement-evoked pain from about day 7 post-surgery through at least day 28 post-surgery, wherein said reduction in pain is at least a 0.5 to 1 point reduction in pain, as measured by an 11 point numerical rating scale, experienced by said patient as compared to a patient not administered the oligonucleotide decoy.

38. The method according to claim 1, wherein the patient experiences a reduction in pain when at rest from about day 7 post-surgery through at least day 28 post-surgery, wherein said reduction in pain is at least a 0.5 to 1 point reduction in pain, as measured by an 11 point numerical rating scale, experienced by said patient as compared to a patient not administered the oligonucleotide decoy.

39-42. (canceled)

43. The method according to claim 1, wherein the patient experiences a reduction in pain from about day 1 post-surgery through at least day 28 post-surgery, as compared to a patient not administered the oligonucleotide decoy, and wherein the magnitude of pain reduction experienced by the patient administered the oligonucleotide decoy is maintained or increases from about day 1 post-surgery through at least day 28 post-surgery, as compared to the patient not administered the oligonucleotide decoy.

44-46. (canceled)

47. The method according to claim 1, wherein the oligonucleotide decoy is formulated as a pharmaceutical composition comprising an in vivo stabilizing amount of a calcium ion.

48-83. (canceled)

84. The method according to claim 1, wherein the single perioperative intrathecal injection is administered into the lumbar region of the patient.


SEQUENCE LISTING (53 sequences)

SEQ ID NO: 1 - DNA, 31 bp, Artificial Sequence
  oligonucleotide decoy
ggcttatgcaaattcgaatgcaaatttgtcg

SEQ ID NO: 2 - DNA, 36 bp, Artificial Sequence
  oligonucleotide decoy
ctaagcccacgtgaccattggccaggtgaccagatc

SEQ ID NO: 3 - DNA, 34 bp, Artificial Sequence
  oligonucleotide decoy
gttatgcgtgggcgataatgcgggggcgttatag

SEQ ID NO: 4 - DNA, 29 bp, Artificial Sequence
  oligonucleotide decoy
gcctccctgagctcattgacgtatctcgg

SEQ ID NO: 5 - DNA, 30 bp, Artificial Sequence
  oligonucleotide decoy
cgaatatgactgagaatgactcagatttgc

SEQ ID NO: 6 - DNA, 37 bp, Artificial Sequence
  oligonucleotide decoy
ggttctatgattttggaatcggattgtgcaaagaagc

SEQ ID NO: 7 - DNA, 34 bp, Artificial Sequence
  oligonucleotide decoy
gcttcaggatgtccatattaggagatcttgttcg

SEQ ID NO: 8 - DNA, 34 bp, Artificial Sequence
  oligonucleotide decoy
ggccacaggatgtaggatgtccatattaggatgc

SEQ ID NO: 9 - DNA, 35 bp, Artificial Sequence
  oligonucleotide decoy
gttctctaaaaataaaaggctaaaaataaaagtcg

SEQ ID NO: 10 - DNA, 30 bp, Artificial Sequence
  oligonucleotide decoy
attaggggcggggtccggggcggggtatta

SEQ ID NO: 11 - DNA, 35 bp, Artificial Sequence
  oligonucleotide decoy
gttatggcggggcggggcggggccgggcggtttac

SEQ ID NO: 12 - DNA, 29 bp, Artificial Sequence
  oligonucleotide decoy
ggcaatgtggttttagtgtggttttacgg

SEQ ID NO: 13 - DNA, 36 bp, Artificial Sequence
  oligonucleotide decoy
gccgtttggggtcatagaaccacaggaaccacacgg

SEQ ID NO: 14 - DNA, 32 bp, Artificial Sequence
  oligonucleotide decoy
cattgcccggaaatggaccggatgtaatttcc

SEQ ID NO: 15 - DNA, 40 bp, Artificial Sequence
  oligonucleotide decoy
gttcttggaaaataaatggaaaatagtggaaaataagtcg

SEQ ID NO: 16 - DNA, 33 bp, Artificial Sequence
  oligonucleotide decoy
cgttcccacttcctgcgaccacttcctgccggg

SEQ ID NO: 17 - DNA, 33 bp, Artificial Sequence
  oligonucleotide decoy
ctgcacctataaatggcctataaatggggatgc

SEQ ID NO: 18 - DNA, 33 bp, Artificial Sequence
  oligonucleotide decoy
gcttatttcgcggaaggtttcccggaagtggcg

SEQ ID NO: 19 - DNA, 31 bp, Artificial Sequence
  oligonucleotide decoy
gctgtgccttatctctttgggataactggcg

SEQ ID NO: 20 - DNA, 32 bp, Artificial Sequence
  oligonucleotide decoy
gcttaatgaataagaggaaaaatgcatgctgg

SEQ ID NO: 21 - DNA, 33 bp, Artificial Sequence
  oligonucleotide decoy
gttctgagattgcacgatgagatttcacagtcg

SEQ ID NO: 22 - DNA, 32 bp, Artificial Sequence
  oligonucleotide decoy
gtcccgcataaataatggcatccttaatcgcg

SEQ ID NO: 23 - DNA, 36 bp, Artificial Sequence
  oligonucleotide decoy
gtgcaggcaagagtagagacaggcaagagtagatgc

SEQ ID NO: 24 - DNA, 25 bp, Artificial Sequence
  oligonucleotide decoy
ccgccaataattaattattaaggcc

SEQ ID NO: 25 - DNA, 35 bp, Artificial Sequence
  oligonucleotide decoy
gcttcgttccatttccggtctcggtttccccattc

SEQ ID NO: 26 - DNA, 34 bp, Artificial Sequence
  oligonucleotide decoy
gctgctgtggaatatcgacctgtggaatatcgtg

SEQ ID NO: 27 - DNA, 37 bp, Artificial Sequence
  oligonucleotide decoy
gccgtataaatgtgctataaaagttttaagaccgtgc

SEQ ID NO: 28 - DNA, 29 bp, Artificial Sequence
  oligonucleotide decoy
gccgtataaatgtgctataaaagccgtgc

SEQ ID NO: 29 - DNA, 26 bp, Artificial Sequence
  oligonucleotide decoy
atgctgcgcttttctccaatctgcgg

SEQ ID NO: 30 - DNA, 40 bp, Artificial Sequence
  oligonucleotide decoy
cgttctccgattggtcacggactctccgattggtcacggc

SEQ ID NO: 31 - DNA, 28 bp, Artificial Sequence
  oligonucleotide decoy
gcgcaccccagcctggctcacccacgcg

SEQ ID NO: 32 - DNA, 37 bp, Artificial Sequence
  oligonucleotide decoy
gatcctttgcctccttcgatcctttgcctccttcaag

SEQ ID NO: 33 - DNA, 29 bp, Artificial Sequence
  oligonucleotide decoy
ggtgtttgggagagctttgggaggatacg

SEQ ID NO: 34 - DNA, 36 bp, Artificial Sequence
  oligonucleotide decoy
gctaatcactcagcatttcggtgagggaagtgaaag

SEQ ID NO: 35 - DNA, 51 bp, Artificial Sequence
  oligonucleotide decoy
cctttcagcaccacggacagcgccagcttcagcaccacggacagcgcctcg

SEQ ID NO: 36 - DNA, 36 bp, Artificial Sequence
  oligonucleotide decoy
ggatcgaacatggagtcagtgagaaatcaggatcgg

SEQ ID NO: 37 - DNA, 35 bp, Artificial Sequence
  oligonucleotide decoy
ggatcgaagccggagtcaaggaggcccctgatcgg

SEQ ID NO: 38 - DNA, 39 bp, Artificial Sequence
  oligonucleotide decoy
ccgaaaggacaaaggtcaagtcgaaaggacaaaggtcag

SEQ ID NO: 39 - DNA, 35 bp, Artificial Sequence
  oligonucleotide decoy
cgggagaaaattcgggaacgttcaagaattgtcgg

SEQ ID NO: 40 - DNA, 34 bp, Artificial Sequence
  oligonucleotide decoy
gttatgcgtgggcgtagatgcgggggcgttatag

SEQ ID NO: 41 - DNA, 16 bp, Artificial Sequence
  oligonucleotide decoy
gatgcgtgggcgtagg

SEQ ID NO: 42 - DNA, 23 bp, Artificial Sequence
  oligonucleotide decoy
gtatgcgtgggcggtgggcgtag

SEQ ID NO: 43 - DNA, 30 bp, Artificial Sequence
  oligonucleotide decoy
gttatgcgtttgtagatgctttcgttatag

SEQ ID NO: 44 - DNA, 20 bp, Artificial Sequence
  oligonucleotide decoy
gttatgcgtgggcgatatag

SEQ ID NO: 45 - DNA, 28 bp, Artificial Sequence
  oligonucleotide decoy
gatgcgtgggcgttgacgtggaaaatgc

SEQ ID NO: 46 - DNA, 30 bp, Artificial Sequence
  oligonucleotide decoy
ctatttcgaaacgatctacattggcataac

SEQ ID NO: 47 - DNA, 22 bp, Artificial Sequence
  oligonucleotide decoy
cgttcccacttcctgcgaccgg

SEQ ID NO: 48 - DNA, 26 bp, Artificial Sequence
  oligonucleotide decoy
gggtgaaggcaagagtagagcggcgg

SEQ ID NO: 49 - DNA, 21 bp, Artificial Sequence
  oligonucleotide decoy
cgttctccgattggtcacgcg

SEQ ID NO: 50 - DNA, 26 bp, Artificial Sequence
  oligonucleotide decoy
gtactccctttgcctccttcaaccgg

SEQ ID NO: 51 - DNA, 31 bp, Artificial Sequence
  oligonucleotide decoy
ccttattcagcaccacggacagcgccattcg

SEQ ID NO: 52 - DNA, 23 bp, Artificial Sequence
  oligonucleotide decoy
gcgaaaggacaaaggtcaggcgg

SEQ ID NO: 53 - DNA, 25 bp, Artificial Sequence
  oligonucleotide decoy
ggcttgctgtggaatatcgatggtg